SEQUENCE LISTING (18 sequences)

SEQ ID NO: 1 - protein, 26 aa, Influenza virus
HAQDILEKEHNGKLCSLKGVRPLILK

SEQ ID NO: 2 - protein, 19 aa, Influenza virus
KEHNGKLCSLKGVRPLILK

SEQ ID NO: 3 - protein, 29 aa, Influenza virus
KKNNAYPTIKRTYNNTNVEDLLIIWGIHH

SEQ ID NO: 4 - protein, 27 aa, Influenza virus
HHSNEQGSGYAADKESTQKAIDGITNK

SEQ ID NO: 5 - protein, 21 aa, Influenza virus
HDSNVKNLYDKVRLQLRDNAK

SEQ ID NO: 6 - protein, 22 aa, Influenza virus
KVRLQLRDNAKELGNGCFEFYH

SEQ ID NO: 7 - protein, 17 aa, Influenza virus
KDVMESMDKEEMEITTH

SEQ ID NO: 8 - protein, 15 aa, Influenza virus
HFQRKRRVRDNMTKK

SEQ ID NO: 9 - protein, 18 aa, Influenza virus
KKWSHKRTIGKKKQRLNK

SEQ ID NO: 10 - protein, 14 aa, Influenza virus
HKRTIGKKKQRLNK

SEQ ID NO: 11 - protein, 26 aa, Influenza virus
HEGIQAGVDRFYRTCKLVGINMSKKK

SEQ ID NO: 12 - protein, 35 aa, Influenza virus
HSWIPKRNRSILNTSQRGILEDEQMYQKCCNLFEK

SEQ ID NO: 13 - protein, 8 aa, West Nile virus
KIIQKAHK

SEQ ID NO: 14 - protein, 10 aa, West Nile virus
HLKCRVKMEK

SEQ ID NO: 15 - protein, 8 aa, West Nile virus
KLTSGHLK

SEQ ID NO: 16 - protein, 13 aa, West Nile virus
HNDKRADPAFVCK

SEQ ID NO: 17 - protein, 10 aa, Foot-and-mouth disease virus
HKQKIIAPAK

SEQ ID NO: 18 - protein, 10 aa, Foot-and-mouth disease virus
HKQKIVAPVK

(19) United States
(12) Patent Application Publication
(10) Pub. No.: US 2009/0269367 A1
(43) Pub. Date: Oct. 29, 2009

(54) METHODS AND COMPOUNDS FOR MITIGATING PATHOGENIC OUTBREAKS USING REPLIKIN COUNT CYCLES

(76) Inventors: Samuel BOGOCH, New York, NY (US); Elenore S. BOGOCH, New York, NY (US); Samuel Winston BOGOCH, Oakland, CA (US); Anne Elenore BORSANYI, Brookline, MA (US)

Correspondence Address: KENYON & KENYON LLP, 1500 K STREET N.W., SUITE 700, WASHINGTON, DC, 20005, US

(21) Appl. No.: 12/429,044

(22) Filed: Apr. 23, 2009

Related U.S. Application Data
(63) Continuation-in-part of application No. PCT/US2008/061336, filed on Apr. 23, 2008.
Application No. PCT/US2008/061336 is a continuation-in-part of application No. 12/108,458, filed on Apr. 23, 2008.
(60) Provisional application No. 61/054,010, filed on May 16, 2008.
(60) Provisional application No. 61/087,354, filed on Aug. 8, 2008.
(60) Provisional application No. 61/143,618, filed on Jan. 9, 2009.

(51) Int. Cl. A61K 39/12; C12Q 1/70; C07K 14/005; C07H 21/04

(52) U.S. Cl. 424/186.1; 435/5; 530/350; 536/23.72; 530/300; 424/204.1

(57) ABSTRACT

The present invention provides methods of predicting increases in pathogenic virulence, morbidity, and/or mortality or expansion in pathogen populations within regions or into new regions by identifying cycles or ratios of increasing concentrations of a family of small peptides expressed in pathogens and provides compounds comprising the small peptides for treatment and prevention of pathogenic outbreaks.

This application claims priority to U.S. Provisional Appln. Ser. No. 61/143,618, filed Jan. 9, 2009, U.S. Provisional Appln. Ser. No. 61/087,354, filed Aug. 8, 2008, U.S. Provisional Appln. Ser. No. 61/054,010, filed May 16, 2008, U.S. application Ser. No. 12/108,458, filed Apr. 23, 2008, and PCT/US2008/61336, filed Apr. 23, 2008, each of which is incorporated herein by reference in its entirety. This application additionally incorporates herein by reference: U.S. application Ser. No. 12/010,027, filed Jan. 18, 2008, U.S. Provisional Appln. Ser. No. 60/991,676, filed Nov. 30, 2007, U.S. application Ser. No. 11/923,559, filed Oct. 24, 2007, U.S. Provisional Appln. Ser. No. 60/982,336, filed Oct. 24, 2007, U.S. Provisional Appln. Ser. No. 60/982,333, filed Oct. 24, 2007, U.S. Provisional Appln. Ser. No. 60/982,338, filed Oct. 24, 2007, U.S. Provisional Appln. Ser. No. 60/935,816, filed Aug. 31, 2007, U.S. Provisional Appln. Ser. No. 60/935,499 filed Aug. 16, 2007, U.S. Provisional Appln. Ser. No. 60/954,743, filed Aug. 8, 2007, U.S. application Ser. No. 11/755,597, filed May 30, 2007, U.S. Provisional Appln. Ser. No. 60/898,097, filed Jan. 30, 2007, U.S. Provisional Appln. Ser. No. 60/880,966, filed Jan. 18, 2007, U.S. Provisional Appln. Ser. No. 60/853,744, filed Oct. 24, 2006, U.S. application Ser. No. 11/355,120, filed Feb. 16, 2006, U.S. application Ser. No. 11/116,203, filed Apr. 28, 2005, U.S. application Ser. No. 10/860,050, filed Jun. 4, 2004, now U.S. Pat. No. 7,442,761, U.S. application Ser. No. 10/189,437, filed Jul. 8, 2002, now U.S. Pat. No. 7,452,963, U.S. application Ser. No. 10/105,232, filed Mar. 26, 2002, now U.S. Pat. No. 7,189,800, U.S. application Ser. No. 09/984,057, filed Oct. 26, 2001, now U.S. Pat. No. 7,420,028, and U.S. application Ser. No. 09/984,056, filed Oct. 26, 2001, now U.S. Pat. No. 7,176,275, each in its entirety.

### BACKGROUND OF THE INVENTION

In surveys of global health, infectious disease often accounts for as many as five of the top ten causes of death in lower- and middle-income countries and respiratory infections are often assigned as the fourth leading cause of death in higher-income countries. Further, pathogenic outbreaks and pandemics continue to threaten human populations from previously unknown or otherwise mutated pathogenic diseases. Previously unknown or otherwise mutated pathogenic diseases often occur when a pathogen diverges from an established host, such as pigs or chickens, into a new host, such as humans. In view of this phenomenon, new strategies are continually needed for mitigating pathogenic outbreaks from previously-known or previously-unknown pathogens. Several such threatening pathogenic diseases include malaria, influenza, West Nile virus, foot-and-mouth disease, and other threats to global health in both humans and animals. Therapies or methods of treatment that are useful across different pathogenic strains or even across pathogenic groups are especially helpful in improving the fight against mutable pathogenic disease and outbreaks of previously-unknown pathogens.

Among the most threatening of global infectious diseases is malaria. Malaria kills a million or more people each year in tropical and sub-tropical environments. Malaria is most commonly and seriously caused by the trypanosome Plasmodium falciparum, which is reportedly responsible for ninety percent of malarial deaths. The majority of death from malarial infection is recorded in young children. P. falciparum is vectored by female Anopheles mosquitoes. Once in the blood stream of a human, the trypanosome multiplies rapidly within red blood cells causing anemia, flu-like symptoms, and sometimes coma and death. Partly effective vaccines are only now beginning to be marketed for malaria and no wholly-effective vaccine has yet been registered for sale in industrialized countries. As such, there continues to be a need in the art for improved methods of predicting and identifying increases in virulence, morbidity, and mortality in and from malaria.

Another threat to public health is West Nile virus (WNV), which causes encephalitis and other serious neuroinvasive diseases in a small percentage of human infections. In about four percent of reported cases, the resulting neuroinvasive disease results in death. WNV is flaviviridae virus, first observed in North America in 1999 and now considered endemic in the United States. The virus is spread to humans through mosquito (and related insect) bites. WNV is a single-stranded sense RNA virus and is a member of the Japanese encephalitis virus antigenic complex, which includes several medically important viruses associated with human encephalitis: Japanese encephalitis, St. Louis encephalitis, Murray Valley encephalitis, and Kunjin encephalitis, an Australian subtype of WNV.

Since introduction of the disease to the United States in 1999, there have been more than 16,000 reported cases of WNV in humans and more than 650 reported deaths. In addition, more than 21,000 cases have been reported in horses. Currently, the only available approved strategies to combat WNV in humans are nationwide active surveillance in conjunction with mosquito control efforts and individual protection with insect repellents. There is a need in the art, therefore, for methods of predicting increases in virulence of WNV prior to epidemics and for therapies for preventing, mitigating, and treating WNV infections.

Influenza is an acute respiratory illness of global importance. Virulent and lethal outbreaks of influenza continue to threaten world health. Researchers, government officials, and medical practitioners are increasingly aware of the continuing threat of a pandemic of virulent and lethal influenza requiring new methods of treatment and novel therapeutic compounds. Researchers, government officials, and medical practitioners likewise recognize that the continuing threat of pandemic influenza requires new and more effective methods of predicting and tracking lethal outbreaks of influenza.

Influenza vaccines remain the most effective defense against influenza virus, but because of the ability of the virus to mutate, and the availability of non-human host reservoirs, it is expected that influenza will remain an emergent or re-emergent infectious threat. Global influenza surveillance indicates that influenza viruses may vary within a country and between countries and continents during an influenza season. Virologic surveillance is of importance in monitoring antigenic shift and drift. Disease surveillance is also important in assessing the impact of epidemics. Both types of information have provided the basis of vaccine composition and use of antivirals. However, traditionally there has been only annual post hoc hematological classification of the increasing number of emerging influenza virus strains, and no specific chemical structure of the viruses was identified as an indicator of approaching influenza epidemic or pandemic. Until the discovery of Replikin chemistry in the virus genome structure, the only basis for annual classification of influenza virus as present or absent in a given year was identification by serological testing of the hemagglutinin and neuraminidase proteins in an isolate of virus. The activity of a strain of influenza was, therefore, only recorded after the fact of the occurrence of the outbreak, never in advance.

There is a continuing need in the art for quantitative methods of tracking and predicting increases in virulence and lethality of influenza prior to outbreaks. There is likewise a need in the art for quantitative methods of preventing and treating outbreaks caused by virulent strains of influenza. Because of the annual administration of influenza vaccines and the short period of time when a vaccine can be administered, strategies directed at improving vaccine coverage are of critical importance.

Replikin peptides are a family of small peptides that have been correlated with the phenomenon of rapid replication in malaria, influenza, West Nile virus, foot and mouth disease, and many other pathogens. Replikin peptides have likewise been correlated with the phenomenon of rapid replication in viruses and organisms generally.

Identification of Replikin peptides has provided targets for detection and treatment of pathogens, including vaccine development against virulent pathogens such as malaria, influenza virus, West Nile virus, and foot and mouth disease virus. In general, knowledge of and identification of this family of peptides enables development of effective therapies and vaccines for any pathogen that harbors Replikins. The phenomenon of the association of Replikins with rapid replication and virulence has been fully described in U.S. Pat. No. 7,189,800, U.S. Pat. No. 7,176,275, U.S. Pat. No. 7,442,761, and U.S. application Ser. No. 11/355,120. Both Replikin concentration (number of Replikins per 100 amino acids) and Replikin composition have been correlated with the functional phenomenon of rapid replication.

There continues to be a need in the art, however, for improved methods of predicting and identifying increases in virulence, morbidity, and lethality and expansions of and outbreaks of virulent pathogens. There is likewise a need in the art for improved methods of preventing and treating outbreaks and expansions of virulent pathogens using Replikin sequences identified with increases in virulence, morbidity, and lethality of expanding pathogenic populations.

### SUMMARY OF THE INVENTION

The present invention provides a quantitative cyclic structure comprising Replikin peptide concentrations identified in a strain of microorganism through time, wherein said cyclic structure correlates in time with the expansion and/or contraction of a population of said strain of microorganism, the infectivity of said strain of microorganism, and/or the lethality of said strain of microorganism.

Further, the present invention provides methods of preventing, mitigating, and treating outbreaks of a pathogen comprising predicting an expansion of a population of a strain of pathogen or an increase in the virulence, morbidity, and/or lethality of a strain of pathogen as compared to another strain of the same or a related pathogen and administering to an animal or patient a compound comprising an isolated or synthesized portion of the structure or genome of the pathogen to mitigate, prevent, or treat the predicted outbreak of the pathogen.

The present invention further provides methods of predicting an expansion of a strain of pathogen or an increase in the virulence, morbidity, and/or mortality of a pathogen comprising identifying a cycle in the Replikin Count in a protein fragment, protein, genome fragment, or genome of a pathogen and predicting an increase in the virulence, morbidity, and/or mortality of said pathogen within the identified cycle in Replikin Count. The present invention further provides Replikin peptides identified within a pathogen predicted to be expanding or to have an increase in virulence, morbidity, and/or mortality as diagnostic, therapeutic, or preventive agents against an outbreak of the pathogen.

A first non-limiting aspect of the invention provides a method of preventing, mitigating, or treating an outbreak of a pathogen predicted to have an expansion of population comprising predicting an expansion of the population of a first pathogen comprising
identifying at least one cycle of Replikin concentration in isolates of the pathogen and predicting that an expansion of the population of the first pathogen will take place after the occurrence of a rising portion of the at least one cycle of Replikin concentration, and
administering to an animal or patient a compound comprising an isolated or synthesized portion of the structure or genome of the pathogen to mitigate, prevent, or treat the predicted outbreak of the pathogen.

A further embodiment of the first aspect of the invention provides a method of preventing, mitigating, or treating an outbreak of pathogen comprising
predicting an expansion of the population or an increase in virulence, morbidity, and/or mortality of an isolate or plurality of isolates of a first strain of pathogen as compared to another isolate or plurality of isolates of the same or a related strain of pathogen comprising: (1) identifying a first cycle in the Replikin concentration of a plurality of isolates of said first strain of pathogen, (2) identifying a first peak in the Replikin concentration within the identified first cycle at a first time point or time period, and (3) predicting an increase in the virulence of an isolate of the same or related strain of pathogen isolated at a second time point or time period subsequent to the first time point or time period; and
administering to an animal or a patient a compound comprising an isolated or synthesized portion of the structure or genome of the at least one isolate of the pathogen to prevent, mitigate, or treat the outbreak of the pathogen.

In a non-limiting embodiment of the first aspect of the invention, the pathogen is an influenza virus, a malarial trypanosome, a West Nile virus, a foot and mouth disease virus, taura syndrome virus, white spot syndrome virus, porcine reproductive and respiratory syndrome virus, porcine circovirus, Helicobacter pylori, Entamoeba invadens, L. legionella, S. aureus, maize streak virus, bovine herpes virus, feline immunodeficiency virus, human immunodeficiency virus, rous sarcoma virus, avian sarcoma virus, sindbis virus, hepatitis virus, b. anthracis, or any other infectious agent. In a non-limiting embodiment, the influenza virus is an H1N1, H2N2, H3N2, H5N1, H3N8, or H9N2 strain of influenza virus.

In a further non-limiting embodiment of the first aspect of the invention, said expansion of a strain of pathogen or increase in virulence, morbidity, and/or mortality of an isolate or plurality of isolates of a strain of pathogen comprises identifying a second cycle in the Replikin concentration of a plurality of isolates of a second strain of pathogen that shares synchrony with said first cycle in the Replikin concentration of said plurality of isolates of said first strain of pathogen and identifying a first peak in the Replikin concentration within the identified first cycle at a first time point or time period and identifying a first peak in the Replikin concentration within the identified second cycle of said second strain of pathogen at a second time point or time period that is similar to said first time point or time period and predicting an increase in the virulence of said first strain of pathogen following the first time point or time period. In a non-limiting embodiment, the pathogen is an influenza virus, a malarial trypanosome, a West Nile virus, a foot and mouth disease virus, or any other infectious agent.

In a non-limiting embodiment of the first aspect of the invention, said pathogen is an influenza virus. In a further non-limiting embodiment, said first strain of influenza is any strain different from said second strain of influenza. In another non-limiting embodiment, said first strain of influenza is H5N1 and said second strain of influenza is H9N2, or vice versa.

In a further non-limiting embodiment of the first aspect of the invention, said isolated or synthesized portion of the structure or genome of the at least one isolate of a pathogen is a protein or protein fragment comprising a Replikin peptide and/or a Replikin Peak Gene, a Replikin peptide identified within a Replikin Peak Gene, or any structure or portion of the structure of said pathogen. In another embodiment, said isolated or synthesized portion of the structure or genome is a nucleic acid encoding a Replikin Peak Gene, a Replikin peptide or a plurality of Replikin peptides within a Replikin Peak Gene, or a Replikin peptide or plurality of Replikin peptides.

In another non-limiting embodiment of the first aspect of the present invention, the second time point or time period is up to three years after the first time point or time period. In a further non-limiting embodiment, the second time point or time period is about one year after the first time point or time period. In a further non-limiting embodiment, the second time point or time period is about six months after the first time point or time period. In a further non-limiting embodiment, the second time point or time period is the next season of a pathogen following the first time point or time period. In a further non-limiting embodiment, the second time point or time period is the next season of influenza following the first time point or first time period. In a further non-limiting embodiment, the next influenza season is the next winter season in a geographic region following the first time point or time period. In another non-limiting embodiment, the second time point or time period is the next season of malaria following the first time point or first time period. In a further non-limiting embodiment, the next season of malaria is the next rainy season. In another non-limiting embodiment, the second time point or time period is the next season of West Nile virus. In a further non-limiting embodiment, the next season of West Nile virus is a summer season.

In another non-limiting embodiment of the first aspect of the present invention, the identified peak in the cycle of Replikin concentration has a higher Replikin concentration than a chronologically earlier peak in the cycle of Replikin concentration. In a further non-limiting embodiment of the invention, the identified peak in the cycle of Replikin concentration is significantly higher than the earlier peak. In a further non-limiting embodiment the identified peak is significantly higher than the earlier peak with a p value less than 0.01. In a further non-limiting embodiment the identified peak is significantly higher than the earlier peak with a p value less than 0.001.

A second non-limiting aspect of the invention provides a method of predicting an expansion of the population of a first pathogen comprising identifying at least one cycle of Replikin concentration in isolates of the pathogen and predicting that an expansion of the population of the first pathogen will take place after the occurrence of a rising portion of the at least one cycle of Replikin concentration, wherein the at least one cycle is cycle A.

In a further embodiment of the second aspect of the invention, the rising portion comprises a peak wherein said expansion of the population of the first pathogen is predicted after the occurrence of the peak. In a further embodiment, the cycle comprises at least a first rising portion and a second rising portion, wherein said first rising portion occurs prior in time to said second rising portion. In a further embodiment, the cycle comprises at least three rising portions, wherein the at least three rising portions are at least rising portion A′, rising portion B′ and rising portion C′. In a further embodiment, the rising portion B′ comprises a peak and the rising portion A′ comprises a peak, and the peak of rising portion B′ has a greater Replikin concentration than the peak of rising portion A′. In a further non-limiting embodiment, the method of prediction further comprises processing the method on a computer. In a further non-limiting embodiment, the cycle comprises more than one cycle including, for example, from peak to trough to peak to trough or from trough to peak to trough to peak. In a further non-limiting embodiment, the cycle comprises three peaks or three troughs or more.

In a further embodiment of the second aspect of the present invention, the method of prediction comprises identifying at least one other cycle of Replikin concentration in isolates of at least one other strain of pathogen, wherein the at least one other cycle is cycle B, and wherein cycle B shares synchrony with cycle A; and predicting that an expansion of the population of the first pathogen will occur after the occurrence of a rising portion in cycle A that corresponds to a rising portion in cycle B. In a further embodiment, the first pathogen is a first strain of influenza virus and the one other pathogen is a different strain of influenza virus. In a further embodiment, the first pathogen is an H5N1 strain of influenza virus and the one other strain of pathogen is an H9N2 strain of influenza virus. In a further embodiment, the expansion of the population of the first pathogen is predicted within three years after the peak. In a further embodiment, the expansion of the population of the first pathogen is predicted within one year after said peak. In a further embodiment, the expansion of the population of the first pathogen is predicted after the next virulence season of the pathogen.

A further embodiment of the second aspect of the invention provides a method of predicting an expansion of a population of a pathogen or an increase in the virulence, morbidity, and/or mortality of a pathogen relative to the population or the virulence, morbidity, and/or mortality of another pathogen of the same species or of another pathogen of a related species comprising: (1) identifying a cycle in the Replikin concentration of isolates of a plurality of the pathogen, (2) identifying a first peak in the Replikin concentration of isolates of a plurality of said pathogen within the identified cycle at a first time point or time period, and (3) predicting an expansion of the population of a pathogen of the same or a related species or an increase in the virulence, morbidity, and/or mortality of a pathogen of the same or a related species isolated at a second time point or time period subsequent to the first time point or time period.

In a non-limiting embodiment of the second aspect of the invention, the pathogen may be, but is not limited to, a malarial trypanosome, West Nile virus, influenza virus, equine influenza virus, coronavirus, foot and mouth disease virus, taura syndrome virus, white spot syndrome virus, or other pathogen or infectious agent.

A non-limiting embodiment of the second aspect of the present invention, the pathogen is a malarial trypanosome. In another non-limiting embodiment, the trypanosome is P. falciparum, P. vivax, P. ovale, or P. malariae. In a further non-limiting embodiment, the trypanosome is P. falciparum. In a further non-limiting embodiment, the method predicts an increase in mortality from malarial infection.

In another embodiment of the second aspect of the present invention, the identified Replikin cycle represents Replikin concentrations identified in a histidine rich protein of P. falciparum. In another non-limiting embodiment of the present invention, the identified Replikin cycle represents Replikin concentrations identified in the histidine-rich protein of P. falciparum.

In another non-limiting embodiment of the second aspect of the present invention, the pathogen is a West Nile virus. In a further embodiment, the identified Replikin cycle represents concentration identified in the envelope protein of West Nile virus. In another non-limiting embodiment, the pathogen is a foot and mouth disease virus. In a further embodiment, the identified Replikin cycle represents concentrations identified in the VP1 protein of foot and mouth disease virus. In another non-limiting embodiment, the pathogen is an influenza virus. In a further embodiment, the identified Replikin cycle represents concentrations identified in the pB1 gene area of influenza virus. In another non-limiting embodiment, the influenza virus is an H1N1, H2N2, H3N2, H3N8, H5N1, or H9N2 strain of influenza virus.

In another non-limiting embodiment of the second aspect of the present invention, the second time point or time period is up to three years after the first time point or time period. In a further non-limiting embodiment, the second time point or time period is about one year after the first time point or time period. In a further non-limiting embodiment, the second time point or time period is about six months after the first time point or time period. In a further non-limiting embodiment, the second time point or time period is the next season of a pathogen following the first time point or time period. In a further non-limiting embodiment, the second time point or time period is the next season of influenza following the first time point or first time period. In a further non-limiting embodiment, the next influenza season is the next winter season in a geographic region following the first time point or time period. In a further non-limiting embodiment, the second time point or time period is following the next dry season after the first time point or time period. In a further non-limiting embodiment, the second time point or time period is the next season of malaria following the first time point or first time period. In a further non-limiting embodiment, the next season is the next rainy season.

In another non-limiting embodiment of the second aspect of the present invention, the identified peak in the cycle of Replikin concentration has a higher Replikin concentration than a chronologically earlier peak in the cycle of Replikin concentration. In a further non-limiting embodiment of the invention, the identified peak in the cycle of Replikin concentration is significantly higher than the earlier peak. In a further non-limiting embodiment the identified peak is significantly higher than the earlier peak with a p value less than 0.01. In a further non-limiting embodiment the identified peak is significantly higher than the earlier peak with a p value less than 0.001.

In a further non-limiting embodiment of the second aspect of the invention, predicting said expansion of population or said increase in virulence, morbidity, and/or mortality of an isolate of a pathogen comprises identifying a second cycle in the Replikin concentration of a plurality of isolates of a second strain or related strain of pathogen that shares synchrony with said first cycle in the Replikin concentration of said plurality of isolates of said first strain of pathogen and identifying a first peak in the Replikin concentration within the identified first cycle at a first time point or time period and identifying a first peak in the Replikin concentration within the identified second cycle of said second strain of pathogen or related strain of pathogen at a second time point or time period that is similar to said first time point or time period and predicting an expansion of the population or an increase in the virulence, morbidity, and/or mortality of said first strain of pathogen following the first time point or time period. In a non-limiting embodiment, the pathogen is a malarial trypanosome, a West Nile virus, a foot and mouth disease virus, or any other infectious agent.

In a non-limiting embodiment, said pathogen is an influenza virus. In a further non-limiting embodiment, said first strain of influenza is any strain different from said second strain of influenza. In another non-limiting embodiment, said first strain of influenza is H5N1 and said second strain of influenza is H9N2, or vice versa. In another embodiment, the strain is any influenza strain and the related strain is any other strain wherein a relationship with said first strain is determined by comparing the Replikin cycles of said strain and said related strain. In another embodiment, the strains are related because the Replikin cycles share synchrony.

A further non-limiting embodiment of the second aspect of the invention provides a method of predicting an expanding population of a pathogen or an increase in virulence, morbidity, and/or mortality in a pathogen comprising: (1) determining the mean Replikin Count in a plurality of isolates of at least two strains of pathogen at a plurality of successive time points; (2) comparing the mean Replikin Count at least four successive time points for each strain and identifying at least one cycle of increasing mean Replikin Counts over the at least four time points for each of the at least two strains; (3) identifying at least partial synchrony between the at least one cycle of increasing mean Replikin Counts for each of the at least two strains; and (4) predicting an increase in virulence following in time the increase in mean Replikin Count in the at least one cycle in said at least two strains wherein said at least one cycle in said at least two strains occurs at a corresponding time period. In a further non-limiting embodiment, step-wise cycles are identified between successive time points. In a further non-limiting embodiment, specific conserved Replikin sequences are identified within the step-wise cycles. In a further non-limiting embodiment, Replikin sequences are identified at the peak of a stepwise cycle. The Replikin sequences identified at the peak of a stepwise cycle are useful for developing a vaccine or therapeutic composition of an isolated or synthesized Replikin peptide for use in preventing or treating outbreaks of malaria with relatively higher mortality. In a further embodiment, the pathogen is influenza. In a further embodiment, the at least two strains of influenza are H9N2 and H5N1.

Another non-limiting embodiment of the second aspect of the invention provides a method of predicting a contraction or failure of a population of a strain of pathogen, wherein an isolate of said pathogen is isolated at a time point or time period subsequent to a decreasing portion of a Replikin cycle.

A further non-limiting embodiment of the second aspect of the invention provides a method for making a vaccine comprising predicting an expanding population of a pathogen or related strain of pathogen or an increase in virulence, morbidity, and/or mortality of a pathogen or a related strain of pathogen and identifying a portion of the structure or genome of said isolated influenza virus to be comprised in a vaccine.

A further non-limiting embodiment of the second aspect of the present invention provides an isolated or synthesized portion of the structure or genome of a pathogen wherein said pathogen is predicted to have an expansion of the population of the pathogen. In a further embodiment, the isolated or synthesized portion is a protein, protein fragment, or peptide comprising a Replikin peptide or a Replikin Peak Gene. In a further non-limiting embodiment, the isolated or synthesized portion of the structure or genome of a pathogen consists of one or more Replikin peptides and/or one or more Replikin Peak Genes. In a further non-limiting embodiment, the one or more Replikin peptides are conserved during a cycle in Replikin concentration at least two successive time points or time periods in the cycle.

Another non-limiting embodiment of the second aspect of the present invention provides Replikin peptides for diagnostic, therapeutic, and/or preventive purposes identified as conserved in an isolate of said pathogen from among a plurality of isolates of said pathogen, wherein said isolates are isolated during a cycle in Replikin concentration at least two successive time points or time periods, and the cycle preferably includes at least two peaks or two troughs.

In a further non-limiting embodiment of the second aspect of the invention, the pathogen is an influenza virus. In a further non-limiting embodiment, the Replikin peptide is at least one of HAQDILEKEHNGKLCSLKGVRPLILK (SEQ ID NO: 1), KEHNGKLCSLKGVRPLILK (SEQ ID NO: 2), KKNNAYPTIKRTYNNTNVEDLLIIWGIHH (SEQ ID NO: 3), HHSNEQGSGYAADKESTQKAIDGITNK (SEQ ID NO: 4), HDSNVKNLYDKVRLQLRDNAK (SEQ ID NO: 5), KVRLQLRDNAKELGNGCFEFYH (SEQ ID NO: 6), KDVMESMDKEEMEITTH (SEQ ID NO: 7), HFQRKRRVRDNMTKK (SEQ ID NO: 8), KKWSHKRTIGKKKQRLNK (SEQ ID NO: 9), HKRTIGKKKQRLNK (SEQ ID NO: 10), HEGIQAGVDRFYRTCKLVGINMSKKK (SEQ ID NO: 11); or HSWIPKRNRSILNTSQRGILEDEQMYQKCCNLFEK (SEQ ID NO: 12).

In a further non-limiting embodiment of the second aspect of the invention, the pathogen is a West Nile virus. In a further non-limiting embodiment, the Replikin peptide is at least one of KIIQKAHK (SEQ ID NO: 13), HLKCRVKMEK (SEQ ID NO: 14), KLTSGHLK (SEQ ID NO: 15), or HNDKRADPAFVCK (SEQ ID NO: 16).

In a further non-limiting embodiment, the pathogen is a foot and mouth disease virus. In a further non-limiting embodiment, the Replikin peptide is at least one of HKQKIIAPAK (SEQ ID NO: 17) and HKQKIVAPVK (SEQ ID NO: 18).

In a further non-limiting embodiment, the pathogen is malaria. In a further non-limiting embodiment, the Replikin peptide is at least one of a Replikin peptide identified from at least one of the following accession numbers: ABU43157, CAD49281, CAD49281, or XP001349534.

Any of the above-listed or herein identified Replikin peptides may be comprised in an immunogenic compound of the invention.

A further non-limiting embodiment of the second aspect of the invention provides a computer readable medium having stored thereon instructions which, when executed, cause a processor to perform a method of predicting an expansion of a strain of pathogen or an increase in virulence, morbidity, and/or mortality of a pathogen. In a further embodiment, the processor reports a prediction to a display, user, researcher, or other machine or person. In a further embodiment, the processor identifies to a display, user, researcher, or other machine or person, a portion of a pathogen predicted to be an expanding pathogen or predicted to increase in virulence, morbidity, and/or mortality, wherein said portion may be employed as a therapeutic or diagnostic compound. Said portion may be a Replikin peptide or plurality of Replikin peptides or any other structure or portion of said genome of said pathogen including a Replikin Peak Gene.

A third non-limiting aspect of the present invention provides Replikin peptides for diagnostic, therapeutic, and/or preventive purposes identified in an isolate of a pathogen, wherein said isolate is isolated during a rising portion of a cycle in Replikin concentration from among a plurality of isolates of the pathogen, or is isolated at a peak in a cycle in Replikin concentration from among a plurality of isolates of a pathogen, or isolated subsequent to a peak in a cycle in Replikin concentration from among a plurality of isolates of a pathogen.

Another non-limiting embodiment of the third aspect of the present invention provides Replikin peptides for diagnostic, therapeutic, and/or preventive purposes identified as conserved in an isolate of a pathogen from among a plurality of isolates said pathogen, wherein said isolates are isolated during a cycle in Replikin concentration at least two successive time points or time periods, and the cycle includes at least two peaks or two troughs.

In a non-limiting embodiment of the third aspect of the present invention, the pathogen is a malarial trypanosome. In a further non-limiting embodiment, the identified cycle is in the histidine rich protein of P. falciparum. In another non-limiting embodiment, the identified cycle is in the ATP-ase protein of P. falciparum. In another non-limiting embodiment, the identified cycle is in a Replikin Peak Gene of a trypanosome that causes malaria.

In another non-limiting embodiment of the third aspect of the present invention, the pathogen is a West Nile virus. In a further non-limiting embodiment, the identified cycle is in the envelope protein of West Nile virus. In another non-limiting embodiment, the pathogen is a foot and mouth disease virus. In a further non-limiting embodiment, the identified cycle is in a VP1 protein of a foot and mouth disease virus.

In another non-limiting embodiment of the third aspect of the present invention, the pathogen is an influenza virus. In another non-limiting embodiment, the influenza virus is an H1N1, H2N2, H3N2, H3N8, H5N1, or H9N2 influenza virus. In a further non-limiting embodiment, the identified cycle is in the neuraminidase or hemagglutinin protein of an influenza virus.

A fourth non-limiting aspect of the present invention provides an immunogenic composition comprising a Replikin peptide identified in an isolate of a pathogen, wherein said isolate is isolated during a rising portion of a cycle in Replikin concentration from among a plurality of isolates of said pathogen, or is isolated at a peak in the identified cycle in Replikin concentration from among a plurality of isolates of the pathogen, or is isolated subsequent to a peak in the identified cycle in Replikin concentration from among a plurality of isolates of the pathogen.

In another non-limiting embodiment of the fourth aspect of the present invention, the immunogenic composition is a vaccine for prevention or treatment of an infection of a pathogen. Another non-limiting embodiment of the present invention provides an antibody to a Replikin peptide identified in an isolate of the pathogen, wherein said isolate is identified during a rising portion of a cycle in Replikin concentration, or is identified at a peak in a cycle in Replikin concentration, or is identified subsequent to a peak in a cycle in Replikin concentration. In another non-limiting embodiment, the pathogen is a West Nile virus. In another non-limiting embodiment, the pathogen is a foot and mouth disease virus.

A fifth non-limiting aspect of the invention provides a method of preventing, mitigating, or treating an outbreak of a pathogen comprising
predicting an expansion of a strain of pathogen comprising (1) determining a mean Replikin Count and a standard deviation of said mean Replikin Count for a plurality of isolates of a strain of pathogen for a first time period in a first geographic region, (2) determining a Replikin Count of at least one isolate of the same or a related strain of pathogen from a second time period and/or second geographic region wherein said second time period is different from said first time period and/or said second geographic region is different from said first geographic region, and (3) predicting an expansion of said strain of pathogen isolated in said second time period and/or second geographic region if the Replikin Count of said at least one isolate is greater than one standard deviation of the mean of the Replikin Count of the plurality of isolates isolated in said first time period and in said first geographic region; and
administering to an animal or a patient a compound comprising an isolated or synthesized portion of the structure or genome of the at least one isolate of influenza virus to prevent or treat the outbreak of influenza virus.

In a non-limiting embodiment of the fifth aspect of the invention, said pathogen is an influenza virus, a malarial trypanosome, a West Nile virus, a foot and mouth disease virus, or any other kind of infectious agent.

In a non-limiting embodiment of the fifth aspect of the invention, said first time period is one year and said first geographic region is a country. In a further embodiment, said second time period is one year. In a further embodiment, said second geographic region is a country. In a further embodiment, where the pathogen is influenza virus, said first geographic region is China. In a further embodiment, where the pathogen is a malarial trypanosome, said first geographic region is India. In a further embodiment, where the pathogen is West Nile virus, said first geographic region is a state within the United States.

In another non-limiting embodiment of the fifth aspect of the invention, said plurality of isolates of a strain of pathogen for a first time period in a first geographic region is a plurality of isolates from all publicly available sequences in said first time period in said first geographic region. In another non-limiting embodiment, said plurality of isolates is all isolates from a species of animal. In another non-limiting embodiment, said plurality of isolates is all isolates from a particular species of bird such as swans, chickens, falcons, turkeys, ducks, or other domestic or wild birds.

In a further non-limiting embodiment of the fifth aspect of the invention, said isolated or synthesized portion of the structure or genome of the at least one isolate of pathogen is a protein or protein fragment comprising a Replikin peptide. In a further embodiment, said protein or protein fragment is a Replikin peptide. In another embodiment, said protein or protein fragment comprises a Replikin Peak Gene. In a further embodiment, said protein or protein fragment is a Replikin Peak Gene. In a further embodiment, said protein or protein fragment is a Replikin peptide identified within a Replikin Peak Gene. In another embodiment, said isolated or synthesized portion of the structure or genome is a nucleic acid encoding a Replikin Peak Gene, a nucleic acid encoding a Replikin peptide or plurality of Replikin peptides within a Replikin Peak Gene, or a nucleic acid encoding a Replikin peptide.

In another non-limiting embodiment of the fifth aspect of the invention, the at least one isolate of the same strain of pathogen from a second time period and/or second geographic region is a plurality of isolates from said second time period and/or second geographic region and the Replikin Count of each isolate of the plurality of isolates from said second time period and/or second geographic region is compared separately to said one standard deviation of said mean Replikin Count.

In a further non-limiting embodiment of the fifth aspect of the present invention, an expansion of said strain of pathogen isolated in said second time period and/or second geographic region is predicted if the number of Replikin Counts of said plurality of isolates from said second period and/or said second geographic region that is greater than one standard deviation of the mean of the Replikin Count of the plurality of isolates isolated in said first time period in said first geographic region, is greater than the number of Replikin Counts of said plurality of isolates from said second time period and/or said second geographic region that is less than said one standard deviation of the mean.

In a further non-limiting embodiment of the fifth aspect of the invention, the Replikin Count is the concentration of Replikin peptides identified encoded in the genome of an isolate of the pathogen. In a further embodiment, the Replikin Count is the concentration of Replikin peptides identified in the expressed proteins of an isolate of the pathogen. In a further embodiment, the Replikin Count is the concentration of Replikin peptides identified in at least one protein or gene area of an isolate of the pathogen. In a further embodiment, the gene area is the pB1 gene area of the genome of influenza virus, the histidine-rich protein gene area of a malarial trypanosome, the VP1 gene area of foot and mouth disease virus, or the envelope protein gene area of West Nile virus. In another embodiment, the Replikin Count is the concentration of Replikin peptides identified in at least one protein fragment of an isolate of the pathogen. In a further embodiment, the Replikin Count is the concentration of Replikin peptides identified in a Replikin Peak Gene of an isolate of the pathogen. In a further embodiment, the Replikin Peak Gene is identified in the polymerase area of an influenza virus genome. In a further embodiment, the Replikin Peak Gene is identified in the pB1 area of an influenza virus genome. In a further embodiment, the Replikin Peak Gene is identified in the histidine-rich protein area of a malarial trypanosome, the VP1 area of a foot and mouth disease virus, or the envelope protein of a West Nile virus.

A sixth non-limiting aspect of the present invention provides a method of predicting an expansion of a strain of pathogen comprising
(1) determining a mean Replikin Count and a standard deviation of said mean Replikin Count for a plurality of isolates of said strain of pathogen for a first time period in a first geographic region;
(2) determining a Replikin Count of at least one isolate of the same or a related strain of pathogen from a second time period and/or second geographic region wherein said second time period is different from said first time period and/or said second geographic region is different from said first geographic region; and
(3) predicting an expansion of said strain of pathogen isolated in said second time period and/or second geographic region if the Replikin Count of said at least one isolate from a second time period and/or second geographic region is greater than one standard deviation of the mean of the Replikin Count of the plurality of isolates isolated in said first time period and in said first geographic region.

In a non-limiting embodiment the method of predicting further comprises processing the method on a computer.

A non-limiting embodiment of the sixth aspect of the invention contemplates that the pathogen is an influenza virus, a malarial trypanosome, a West Nile virus, a foot and mouth disease virus, or any other kind of infectious agent.

A further non-limiting embodiment of the sixth aspect of the invention provides a method for making a vaccine comprising predicting an expansion of said strain of pathogen isolated in said second time period and/or second geographic region and identifying a portion of the structure or genome of said isolated influenza virus to comprise a vaccine.

In a further non-limiting embodiment of the sixth aspect of the invention, the at least one isolate of the same strain of pathogen from a second time period and/or second geographic region is a plurality of isolates from said second time period and/or second geographic region. In a further non-limiting embodiment, the Replikin Count of each isolate of the plurality of isolates from said second time period and/or second geographic region is compared separately to said one standard deviation of the mean.

In another non-limiting embodiment of the sixth aspect of the invention, an expansion of a strain of pathogen isolated in said second time period and/or said second geographic region is predicted if the number of Replikin Counts of said plurality of isolates from said second time period and/or said second geographic region that is greater than one standard deviation of the mean of the Replikin Count of the plurality of isolates isolated in said first time period in said first geographic region, is greater than the number of Replikin Counts of said plurality of isolates from said second time period and/or said second geographic region that is less than said one standard deviation of the mean. In a further non-limiting embodiment, an expansion of a strain of influenza virus isolated in said second time period and/or second geographic region is predicted if the ratio of the number of Replikin Counts of said plurality of isolates from said second time period and/or said second geographic region that is greater than said one standard deviation of the mean, divided by the number of Replikin Counts of said plurality of isolates from said second time period and/or said second geographic region that is less than said one standard deviation of the mean, is greater than one.

A further non-limiting embodiment of the sixth aspect of the present invention provides Replikin peptides for diagnostic, therapeutic, and/or preventive purposes identified in an isolate of a pathogen predicted to have an expanding population. In another non-limiting embodiment, the Replikin peptides for diagnostic, therapeutic, and/or preventive purposes are conserved over time or across geographic regions.

Another non-limiting embodiment of the sixth aspect of the invention provides a method of predicting a contraction or failure of a population of a strain of pathogen, wherein a Replikin Count of at least one isolate of a strain of pathogen from a first time period and/or first geographic region is less than one standard deviation of the mean of the Replikin Count of a plurality of isolates of influenza from a second time period and second geographic region. Another non-limiting embodiment provides a method of predicting a contraction or failure of a population of a strain of pathogen, wherein the number of Replikin Counts of a plurality of isolates from a first time period and/or a first geographic region greater than one standard deviation of the mean of the Replikin Count of a plurality of isolates from a second time period in a second geographic region, is less than the number of Replikin Counts of the plurality of isolates from the first time period and/or the first geographic region that is less than said one standard deviation of the mean. In a further non-limiting embodiment, said contraction or failure is predicted if the ratio of the number of Replikin Counts of said plurality of isolates from said first time period and/or said first geographic region that are greater than said standard deviation of the mean, divided by the number of Replikin Counts of said plurality of isolates from said first time period and/or said first geographic region that are less than said standard deviation of the mean, is less than one.

A further non-limiting embodiment of the sixth aspect of the invention provides a computer readable medium having stored thereon instructions which, when executed, cause a processor to perform a method of predicting an expansion of a strain of pathogen or the expansion of a virus or organism. In a further embodiment, the processor reports a prediction to a display, user, researcher, or other machine or person. In a further embodiment, the processor identifies to a display, user, researcher, or other machine or person, a portion of a pathogen predicted to be an expanding pathogen, wherein said portion may be employed as a therapeutic or diagnostic compound. Said portion may be a Replikin peptide or plurality of Replikin peptides or any other structure or portion of said genome of said pathogen including a Replikin Peak Gene.

A seventh non-limiting aspect of the present invention provides an immunogenic composition comprising a portion of the structure or genome of an isolate of a pathogen, wherein said isolate of said pathogen is (1) an isolate having a Replikin Count greater than one standard deviation of a mean Replikin Count of a plurality of isolates of pathogen isolated in a different time period and/or in a different geographical region, (2) an isolate from a first time period and/or geographical region wherein the number of a plurality of isolates from the first time period and/or geographical region having a Replikin Count greater than said one standard deviation of the mean is greater than the number of isolates having a Replikin Count less than said one standard deviation of the mean, (3) isolated during a rising portion of a cycle or a set of two or more synchronous cycles in Replikin concentration from among a plurality of isolates of influenza, and/or (4) isolated at a peak in the identified cycle or set of synchronous cycles in Replikin concentration from among a plurality of isolates of influenza.

In another non-limiting embodiment the seventh aspect of the present invention, the immunogenic composition is a vaccine for prevention or treatment of an infection of a pathogen. Another non-limiting embodiment provides an antibody to a Replikin peptide identified in an isolate of pathogen predicted to have an increase in virulence, morbidity, and/or lethality or expansion of its population.

### BRIEF DESCRIPTION OF THE DRAWINGS

FIG. 1 illustrates cycling between 1986 and 2007 of mean annual Replikin concentration in the histidine rich protein of Plasmodium falciparum for sequences available at www.pubmed.com for isolates from 1986 through 2007. In FIG. 1, three rising portions of cycles of Replikin concentration and two decreasing portions of cycles of Replikin concentration are observable with peaks at 1987 and 1999. A first rising portion and decreasing portion of a cycle is observed from 1986 to 1995. A second rising portion and decreasing portion of a cycle is observed from 1996 to 2005. A new cycle appears to have begun between 2005 and 2007. The peak of the first rising portion was identified in 1987 with a mean annual Replikin Count of 38.2 and standard deviation of ±23.5. The peak of the second rising portion was identified in 1999 with a step-wise even higher mean annual Replikin Count of 62.9 and standard deviation of ±63. Both the 1987 peak and the 1999 peak were observed to be related to higher human mortality. Following the 1999 peak, mean annual Replikin Counts were observed to fall to a low of 7.4 in 2005 with a standard deviation of ±6.5. Mortality rates likewise fell between 2000 and 2005. A new malaria Replikin cycle appears to have begun in 2005 with the observed mean annual Replikin Count increasing from 7.4±6.5 in 2005 to 17.2±19 in 2007. The beginning of the new cycle provides a prediction that Replikin Count may continue to increase along with an increase in malaria mortality rate.

FIG. 2 illustrates that mortality rates per 1000 clinical cases of malaria in humans generally correlate with mean annual Replikin Count in sequences of the P. falciparum ATP-ase enzyme publicly available at www.pubmed.com. Mean annual Replikin Counts of P. falciparum ATP-ase increased from 1997 to 1998 along with an increase in mortality per malaria case from 1997 and 1998 to 1999. The mean annual Replikin Count of P. falciparum ATP-ase decreased from 1998 to 2006 along with the mortality rates from 1999 to 2005 (consistent mortality data is considered presently available only through 2005). The data for FIG. 2 may be seen in Table 6 below. Mortality rates in FIG. 2 and Table 6 are recorded as declared by the World Health Organization. See www.who.int.

FIG. 3 illustrates cycling of mean annual Replikin Count in West Nile virus in correlation with cycling of West Nile virus morbidity. The mean annual Replikin Count of the Envelope Protein of WNV (black) and standard deviation (capped line) is compared to the annual number of human cases in the United States as reported by the Centers for Disease Control (CDC) (gray). Mean annual Replikin Count was analyzed in envelope protein sequences from isolates isolated between 2000 and 2006 and publicly available at www.pubmed.com. In FIG. 3, the standard deviation of the mean of the Replikin Count of the envelope protein is observed to increase markedly from 2000 to 2001 (p<0.001). This change has been observed to signal rapid replication and expansion of the range of the Replikin Count preceding virus outbreak in all common strains of influenza virus (not the same virus genus as WNV) as standard deviation within a virus population increases. The increase in mean Replikin Count in WNV from 2000 to 2003 appears to accompany, or precede, the increase in the number of human WNV cases recorded independently and published by the CDC. A decrease in mean annual Replikin Count and recorded human cases of WNV is observed following 2003. In 2006, an increase is observed in the Replikin Count followed by an increase in 2007 of the number of human cases. As a result, FIG. 3 illustrates two rising portions and one decreasing portion in a cycle of Replikin concentration and two rising portions and one decreasing portion in a cycle of WNV human morbidity, the first rising portion from 2000 to 2003 and the second rising portion from 2004 to 2006/2007. Conserved viral Replikin structures within the envelope protein are observed throughout the illustrated cycles and the relationship between Replikin structure and rapid replication and virulence are observed through time.

FIG. 4 illustrates cycling in Replikin concentration in the whole genome of foot and mouth disease virus (FMDV) type O isolated between 1999 and 2008 and reported at www.pubmed.com. The data demonstrate that annual Replikin Counts (Mean and Standard Deviation (SD)) for isolates of FMDV type O occurred with two rising portions and one decreasing portion. A first rising portion and a first decreasing portion are observed between 1999 and 2005. A second rising portion is observed beginning in 2005 through 2008. The cycle is presently incomplete since a second trough is not yet observable. In FIG. 4, mean annual Replikin Count is observed to provide advance warning signals (with p<0.001) prior to severe FMDV outbreaks in the U.K. and the Netherlands in 2001-2002, Mean annual Replikin Count is further observed to provide advance warning signals (with p<0.001) prior to severe FMDV outbreaks in the Middle East, Africa, India, and Asia (including China) in 2008-2009. Replikin cycles are detectable because of repeating conserved virus structures and continuity of the Replikin phenomenon through time. The data in FIG. 4 demonstrate that the highest mean annual Replikin Counts over the ten year period reflected in FIG. 4 were observed in 2007 and 2008.

FIG. 5 illustrates cycles of mean annual Replikin Count in influenza sequences from the pB1 gene area for isolates isolated between 1993 and 2008 and reported at www.pubmed.com. In FIG. 5, the mean annual Replikin Count of the pB1 gene area of isolates of H9N2 is shown in light gray columns with standard deviation shown above in dark gray columns. The number of poultry flocks reported in Israel with H9N2 infection is provided in white columns. The data illustrate an increase in mean annual Replikin Count that corresponds to an increase in influenza outbreaks in flocks of poultry in Israel between 2000 and 2004. The standard deviation data further emphasize the extent of expanding Replikin Counts within the annual H9N2 influenza population.

FIG. 6 illustrates synchronous cycles of mean annual Replikin Counts in the pB1 gene area of H9N2 and H5N1 influenza isolates. The data represent analysis of sequences of isolates isolated between 1993 and 2008 and reported at www.pubmed.com. In FIG. 6, annual mean Replikin Count for H9N2 is reported in light gray columns with standard deviation reported above in dark gray columns. Annual mean Replikin Count for H5N1 is reported in black columns with standard deviation reported above in white columns. FIG. 6 visibly illustrates synchrony between the H9N2 and H5N1 Replikin Cycles. The synchronous cycles individually and together predict H5N1 outbreaks in Hong Kong in 1997, 2002, 2004, 2007, and a present outbreak of H5N1 and H9N2 in 2008-2009. Because the cycles of different strains correspond with a level of synchrony, the predictive capacity of the individual cycles is increased by the correspondence. Further an interrelationship between H5N1 and H9N2 is demonstrated suggesting that H9N2 may be a candidate for a future influenza pandemic just as H5N1 has been known to be a candidate for such a pandemic.

FIG. 7 illustrates cycling in mean annual Replikin Counts in the pB1 gene area in the three influenza pandemics of the last century. Strain-specific high Replikin Counts accompany each of the three pandemics: 1918, 1957, and 1968. In each case, a first peak is followed by a decline (likely due to immunity in the hosts), then by a second recovery peak and a “rebound” epidemic. The probability is very low that these correlations are due to chance, since they are specific for each strain, specific for each of the three pandemic years out of the century, specific for each post-pandemic decline, and specific for each rebound epidemic. The data supports a prediction of an increase in virulence and morbidity following a peak in a cycle of mean annual Replikin Count in influenza virus. For influenza strains that result in mortality, an increase in virulence and morbidity was accompanied by increased mortality in the pandemics of the 20^{th} Century.

FIG. 8 illustrates the same data as FIG. 7 but is expanded in size for better viewing of the data for individual years. FIG. 8(A-C) illustrates cycles in Replikin Count in strains of influenza related to outbreaks of influenza between 1917 and 2007. The data illustrate an increase in Replikin Count before and accompanying each influenza A pandemic and outbreak since 1918 and low Replikin Counts during quiescent periods of influenza A infection and continually in non-lethal Influenza B. The graph provides annual Replikin Counts from 1917-2007 for all Replikin Peak Genes isolated in silico in the pB1 gene area of influenza strains having amino acid or nucleic acid sequences publicly available at PubMed. Data is provided (1) for non-lethal human Influenza B between 1940 and 2007 (thick solid line) and (2) for both the lethal and non-lethal periods of human Influenza A viruses between 1917 and 2007. Human Influenza A strains are (1) H1N1 (thin solid line), (2) H2N2 (long-short-long dashed line), (3) H3N2 (medium dashed line), and (4) H5N1 (long dashed line). H5N1 strains isolated from chicken are illustrated by a short dashed line. The total number of sequences analyzed for the data (N) is 14,227. Listed pandemics, epidemics and outbreaks are the 1918 H1N1 pandemic, the 1930's H1N1 epidemic, the 1957H2N2 pandemic, the 1968H3N1 pandemic, the 1977-78H3N2 outbreaks and the H5N1 outbreaks of 2001-2004 and 2007. A 1997 outbreak of H5N1 is not shown in FIGS. 7 and 8. Over a ninety year period, pandemics, epidemics and outbreaks are associated with Replikin Counts of four or above in the RPG of influenza strains. Over the same period, constant low Replikin Counts of less than four may be observed during quiescent non-lethal periods of influenza A infections and low Replikin Counts of less than four may be observed in non-lethal Influenza B.

FIG. 9 illustrates an immune response with protective effect following administration of a vaccine comprising a mixture of peptides of SEQ ID NO(s): 1-12 to chickens later challenged with Low-Path H5N1 virus. Eighty chickens were divided into four groups of twenty chickens each on a first day after hatch. Group 1 was a negative control subjected to neither vaccination nor infection with the Low-Path H5N1 virus. Group 2 was a vaccine control subjected to vaccination intranasally on day 1 after hatch, intraocularly on day 7 after hatch, and via spray inhalation on day 14 after hatch. Group 2 was not subjected to infection with the Low-Path H5N1 virus. Group 3 was subjected to vaccination on the same schedule as Group 2 and Low-Path H5N1 was introduced to the cleft palate of the chickens on day 28. Group 4 was a challenged control that was not vaccinated but was infected with H5N1 on day 28 via the cleft palate. On days 7, 14, and 21, between six and nine chickens from each group were tested for serum production of antibodies against H5N1 virus. The data from the serum antibody tests are contained in Table 15 and illustrated in FIG. 9. FIG. 9 illustrates that only one of seven (14%) chickens tested in Group 3 (vaccinated and challenged with virus) was observed to produce antibody in serum seven days after challenge while four of seven chickens (57%) tested in Group 4 (not vaccinated but challenged) were observed to produce antibody in serum seven days after challenge. FIG. 9 further illustrates that only three of six chickens (50%) tested in Group 3 (vaccinated and challenged) were observed to produce antibody in serum fourteen days after challenge while seven of nine (78%) chickens tested in Group 4 (not vaccinated but challenged) were observed to produce antibody in serum fourteen days after challenge. FIG. 9 further illustrates that two of seven (29%) chickens tested in Group 3 were observed to produce antibody in serum twenty-one days after challenge while three of nine (33%) chickens tested in Group 4 were observed to produce antibody in serum twenty-one days after challenge. In the vaccine control (Group 2) six of six (100%) chickens tested were observed to produce antibody in serum fourteen days after challenge while no chickens tested on days 7 or 21 were observed to produce antibody in serum. In the negative control (Group 1), no chickens were observed to produce antibody in serum on any day of testing. In combination with data provided in Example 10 demonstrating that no H5N1 virus was observed by PCR detection excreted in feces or saliva from chickens in Groups 1, 2, and 3 (negative control, vaccine control, a vaccine/challenge groups, respectively) and that H5N1 virus was observed by PCR detection excreted in feces and saliva for all chickens in Group 4 (challenge control), one of ordinary skill in the art concludes that chickens in the vaccinated groups (Groups 2 and 3) produced an immune response to the vaccine and that chickens in the vaccinated and challenged group (Group 3) were provided a measure of protection from the Low-Path H5N1 challenge on day 28 following hatch.

### DETAILED DESCRIPTION OF THE INVENTION

### Definitions

As used herein, a “Replikin cycle” or “a cycle of Replikin concentration” or “a cycle of Replikin Count” means Replikin concentrations of a plurality of isolates of a species of virus or organism wherein at least four of said plurality of isolates are isolated at successive time points or in successive time periods, wherein a Replikin concentration of a second individual isolate or second mean of a plurality of isolates at a second time point or time period is higher than a Replikin concentration of a first individual isolate or a first mean of a plurality of isolates at a first time point or time period, a Replikin concentration of a third individual isolate or a third mean of a plurality of isolates at a third time point or time period is lower than the Replikin concentration at a second time point or time period, and a Replikin concentration of a fourth individual isolate or fourth mean of a plurality of isolates at a fourth time point or time period is higher than the Replikin concentration at a third time point or time period; or wherein a Replikin concentration of a second individual isolate or second mean of a plurality of isolates at a second time point or time period is lower than a Replikin concentration of a first individual isolate or a first mean of a plurality of isolates at a first time point or time period, a Replikin concentration of a third individual isolate or a third mean of a plurality of isolates at a third time point or time period is higher than the Replikin concentration at a second time point or time period, and a Replikin concentration of a fourth individual isolate or fourth mean of a plurality of isolates at a fourth time point or time period is lower than the Replikin concentration at a third time point or time period. Within the Replikin cycle, cycle of Replikin concentration, or cycle of Replikin Count, the second time point or time period must be later in time than the first time point or time period, the third time point or time period must be later in time than the second time point or time period, and the fourth time point or time period must be later in time than the third time point or time period. Within a Replikin cycle, any rising portion is predictive of an expansion in population or an increase in virulence, morbidity, and/or mortality of a pathogen in hosts and any decreasing portion is predictive of a contracting population or a decrease in virulence, morbidity, and/or mortality of pathogen in hosts. A cycle need not be complete to be predictive, a decreasing portion followed by a rising portion is predictive of an expanding population or an increase in virulence, morbidity, and/or mortality. Likewise, a rising portion followed by a decreasing portion followed by a rising portion is predictive of an expanding population or an increase in virulence, morbidity, and/or mortality. As such, cycles need not be complete cycles to provide predictive capacity concerning an expansion or contraction (or change in virulence, morbidity, and/or mortality) of a pathogen in hosts.

As used herein, a “step-wise” cycle is any set of cycles wherein a first Replikin cycle peak in time is lower than a second Replikin cycle peak in time or a first Replikin cycle peak in time is higher than a second Replikin cycle peak in time. A step-wise cycle also occurs when successive peaks are observed to move lower. A step-wise cycle may also be observed if successive troughs move higher or lower. Step-wise cycles provide additional predictive capacity for predictions of expansion or contraction of a population.

As used herein a Replikin cycle that is “synchronous,” shares “synchrony,” or any other related word, with another Replikin cycle means a cycle having a period or phase or any portion of the cycle that is similar to some period, phase, or portion of the cycle wherein said similarity may be determined visually, mathematically, statistically, or by any other method known or hereinafter known by one of skill in the art. Synchronous cycles do not necessarily share portions that arise or occur at exactly the same time. Synchronous cycles in related pathogens will at times be shifted by some measure of time from one another and may shift in time from one another in any portion of either cycle. A portion of a Replikin cycle “corresponds” in time with another Replikin cycle if there is a similarity between the portions of the cycle. Any correspondence need not be exact.

As used herein, a “rising portion” of a Replikin cycle means the Replikin concentration of an isolate or the mean Replikin concentration of a plurality of isolates, wherein the isolate or isolates were isolated at a time point or time period of the Replikin cycle where the trend of Replikin concentration in the Replikin cycle is increasing from at least a first time point or time period to at least a second time point or time period. Additionally, the rising portion may include a peak.

As used herein, a “decreasing portion” of a Replikin cycles means the opposite of a rising portion, wherein a decreasing portion may include a trough.

As used herein, a “peak” in a Replikin cycle means a second time point or time period within a Replikin cycle, wherein the Replikin concentration at a first time point or time period sequentially preceding the second time point or time period is lower than the Replikin concentration at the second time point or time period, and the Replikin concentration at a third time point or time period sequentially following the second time point or time period is lower than the Replikin concentration at the second time point or time period. One of skill in the art will understand that because of the variability of biological systems, a peak may include a general region of a cycle that is generally higher than a sequentially preceding region and generally higher than a sequentially following region rather than an exact time point or time period.

As used herein, a “trough” in a Replikin cycle means the opposite of a peak in a Replikin cycle.

As used herein, a “Replikin Count Virus Expansion Index” or “RCVE Index” or a “Replikin Count Expansion Index” or “RCE Index” is the number of Replikin Counts of a plurality of isolates from a first time period and/or first geographic region that are greater than one standard deviation of the mean of the Replikin Count of a plurality of isolates isolated in a second time period and in a second geographic region, divided by the number of Replikin Counts of said plurality of isolates from said first time period and/or said first geographic region that are less than one standard deviation of the mean of the Replikin Count of the plurality of isolates isolated in said second time period in said second geographic region. An RCE or RCVE Index predicts the expansion of a pathogen in a particular region and/or time period if the ratio of the RCE or RCVE Index is greater than one. An RCE or RCVE Index predicts the contraction, retraction, reduction, or failure of a pathogen in a particular region and/or time period if the ratio of the RCE or RCVE Index is less than one. An RCE or RCVE Index predicts equilibrium between expansion and contraction in the pathogen population if the ratio of the RCVE Index is equal to one.

As used herein, a “related pathogen” means a first pathogen that is of the same species, genus, or family as a second pathogen for which a relationship is known now or hereafter by one of skill in the art. A related pathogen may be a first pathogen that is of the same species but a different strain from a second pathogen. A related pathogen may be a first pathogen that is the same or different species from a second pathogen and shares a host, reservoir, or vector with the second pathogen. Even if a first pathogen is not of the same species, genus, or family as a second pathogen, the first pathogen is related to the second pathogen if the first pathogen has a Replikin cycle that is synchronous with the Replikin cycle of the second pathogen. One of skill in the art will recognize the many ways that a first pathogen may be related to a second pathogen. A related pathogen may be within the same family as a first pathogen. A related pathogen may be within the same genus as a first pathogen. A related pathogen may be within the same species as a first pathogen. A related pathogen may be within the same strain as a first pathogen.

As used herein, different “time periods” or different “time points” are any two time periods or time points that may be differentiated from each other. For example, an isolate of an organism or virus isolated during the year 2004 may be considered to be isolated in a different time period than an isolate of the same organism or virus isolated during the year 2005. Likewise, an isolate of an organism or virus isolated in May 2004 may be considered to be isolated in a different time period than an isolate of the same organism or virus isolated in June 2004. When comparing Replikin concentrations of different isolates, one may use comparable time periods. For example, an isolate from 2004 may be compared to at least one other isolate from some other year such as 2002 or 2005. Likewise, an isolate from May 2004 may be compared to at least one isolate from some other month of some year, for example, an isolate from December 2003 or from June 2004.

As used herein, an “isolate” is any virus or organism isolated from a natural source wherein a natural source includes, but is not limited to, a reservoir of an organism or virus, a vector of an organism or virus, or a host of an organism or virus. “Obtaining,” “isolating,” or “identifying” an isolate is any action by which an amino acid or nucleic acid sequence within an isolate is obtained including, but not limited to, isolating an isolate and sequencing any portion of the genome or protein sequences of the isolate, obtaining any nucleic acid sequence or amino acid sequence of an isolate from any medium, including from a database such as PubMed, wherein the nucleic acid sequence or amino acid sequence may be analyzed for Replikin concentration, or any other means of obtaining the Replikin concentration of a virus isolated from a natural source at a time point or within a time period.

As used herein, “an earlier-arising” virus or organism or a virus or organism isolated at “an earlier time point” or during “an earlier time period” is a specimen of a virus or organism collected from a natural source of the virus or organism on a date prior to the date on which another specimen of the virus or organism was collected from a natural source. A “later-arising” virus or organism or a virus or organism isolated at a “later time point” or during a “later time period” is a specimen of a virus or organism collected from a natural source of the virus (including, but not limited to, a reservoir, a vector, or a host) or a natural source of the organism on a date subsequent to the date on which another specimen of the virus or organism was collected from a natural source.

As used herein, the “next virulence season” of a pathogen is a time period in which an increase in morbidity of a pathogen is expected based on seasonal changes, such as a change from summer to winter or a change from a wet season to a dry season, wherein the pathogen was experiencing less morbidity in a previous sequential time period prior to the time period in which the increase in morbidity is expected to occur.

As used herein, the term “dry season” or “winter season” with respect to malaria describes a season in any geographical region wherein mosquito activity (including feeding and reproduction) is significantly less than during other times of the year. A peak in a Replikin cycle before a dry season or winter season predicts an increase in virulence, morbidity, and/or mortality in malaria in the following rainy season or summer season when mosquito activity is greatest.

As used herein “trypanosome that causes malaria,” “malarial trypanosome” or “trypanosome” in singular or plural means any Plasmodium species or other species known now or hereafter to cause malaria. Malarial trypanosomes include but are not limited to Plasmodium falciparum, Plasmodium vivax, Plasmodium ovale, and Plasmodium malariae.

As used herein, a “Replikin Peak Gene (RPG)” (or sometimes a Replikin Peak Gene Area-RPGA) means a segment of a genome, protein, segment of protein, or protein fragment in which an expressed gene or gene segment has a highest concentration of continuous, non-interrupted and overlapping Replikin sequences (number of Replikin sequences per 100 amino acids) when compared to other segments or named genes of the genome. Generally, a whole protein or gene or gene segment that contains the amino acid portion having the highest concentration of continuous Replikin sequences is also referred to as the Replikin Peak Gene. More than one RPG may be identified within a gene, gene segment, protein, or protein fragment. An RPG may have a terminal lysine or a terminal histidine, two terminal lysines, or a terminal lysine and a terminal histidine. For diagnostic, therapeutic and preventive purposes, an RPG may have a terminal lysine or a terminal histidine, two terminal lysines, or a terminal lysine and a terminal histidine or may likewise have neither a terminal lysine nor a terminal histidine so long as the terminal portion of the RPG contains a Replikin sequence or Replikin sequences defined by the definition of a Replikin sequence, namely, an amino acid sequence having about 7 to about 50 amino acids comprising:
(1) at least one lysine residue located six to ten amino acid residues from a second lysine residue;
(2) at least one histidine residue; and
(3) at least 6% lysine residues.
   Further, for diagnostic, therapeutic, preventive and predictive purposes, an RPG may include the protein or protein fragment that contains an identified RPG. For predictive purposes, a Replikin Count in the RPG may be used to track changes in virulence and lethality. Likewise the RPG may be used as an immunogenic compound or as a vaccine. Whole proteins or protein fragments containing RPGs are likewise useful for diagnostic, therapeutic and preventive purposes, such as, for example, to be included in immunogenic compounds, vaccines and for production of therapeutic or diagnostic antibodies.

As used herein, a “Replikin sequence” is an amino acid sequence of 7 to about 50 amino acids comprising or consisting of a Replikin motif wherein the Replikin motif comprises:
(1) at least one lysine residue located at a first terminus of said isolated peptide and at least one lysine residue or at least one histidine residue located at a second terminus of said isolated peptide;
(2) a first lysine residue located six to ten residues from a second lysine residue;
(3) at least one histidine residue; and
(4) at least 6% lysine residues.
   For the purpose of determining Replikin concentration, a Replikin sequence must have a lysine residue at one terminus and a lysine or a histidine residue at the other terminus. For diagnostic, therapeutic, and preventive purposes, a Replikin sequence may or may not have defined termini.

The term “Replikin sequence” can also refer to a nucleic acid sequence encoding an amino acid sequence having about 7 to about 50 amino acids comprising:
(1) at least one lysine residue located six to ten amino acid residues from a second lysine residue;
(2) at least one histidine residue; and
(3) at least 6% lysine residues,
   wherein the amino acid sequence may comprise a terminal lysine and may further comprise a terminal lysine or a terminal histidine.

As used herein, the term “peptide” or “protein” refers to a compound of two or more amino acids in which the carboxyl group of one amino acid is attached to an amino group of another amino acid via a peptide bond. As used herein, “isolated” or “synthesized” peptide or biologically active portion thereof refers to a peptide that is, after purification, substantially free of cellular material or other contaminating proteins or peptides from the cell or tissue source from which the peptide is derived, or substantially free from chemical precursors or other chemicals when chemically synthesized by any method, or substantially free from contaminating peptides when synthesized by recombinant gene techniques or a protein or peptide that has been isolated in silico from nucleic acid or amino acid sequences that are available through public or private databases or sequence collections. An “encoded” or “expressed” protein, protein sequence, protein fragment sequence, or peptide sequence is a sequence encoded by a nucleic acid sequence that encodes the amino acids of the protein or peptide sequence with any codon known to one of ordinary skill in the art now or hereafter. It should be noted that it is well-known in the art that, due to redundancy in the genetic code, individual nucleotides can be readily exchanged in a codon and still result in an identical amino acid sequence. As will be understood by one of skill in the art, a method of identifying a Replikin amino acid sequence also encompasses a method of identifying a nucleic acid sequence that encodes a Replikin amino acid sequence wherein the Replikin amino acid sequence is encoded by the identified nucleic acid sequence.

As used herein, “outbreak” is an increase in virulence, morbidity, and/or mortality in a pathogenic disease or an expansion in the population of pathogen as compared to a baseline of an earlier occurring epidemiological pattern of infection in the same disease. One of ordinary skill in the art will know how to determine an epidemiological baseline.

As used herein, “morbidity,” is the number of cases of a disease caused by the virus, either in excess of zero cases in the past or in excess of a baseline of endemic cases in the past. Therefore the baseline of endemic cases, in epidemiological terms, may, for example, relate to whether none or some cases were present in a geographic region in the immediate past. The past, in epidemiological terms, may mean more than one year and can mean several years or more as understood by one of ordinary skill in the art. The past may also mean less than one year as determined by one of ordinary skill in the art. In the case of annually-recurrent common influenza and seasonal malaria and West Nile virus, for example, the baseline often reflects an annual recurrence or expansion and contraction of these diseases.

As used herein, “expansion” of a pathogen or a population of pathogen and “expanding” pathogen or population of pathogen means an increase in virulence, morbidity, and/or lethality of a pathogen (e.g., strain of P. falciparum, a strain of influenza virus, etc.) and/or an expansion of the population of a pathogen (e.g., strain of P. falciparum, a strain of influenza virus, etc.) wherein said expansion includes an increase in the occurrence of the pathogen in a given geographic region or in a given time period or both, or a spreading of the occurrence of the pathogen to another geographic region.

As used herein, an increase or decrease in “virulence” includes an increase or decrease in virulence, morbidity, lethality, host mortality, and/or expansion of a pathogen, such as an influenza virus.

As used herein, “geographic region” or similar term is an area differentiated from another area by space. For example, China is a geographic region that may be differentiated from the geographic region of India. Likewise a geographic region may be a town, or city, or continent or any area differentiable from another area. A geographic region may encompass the entire earth if an isolate or plurality of isolates from a given time period is compared to isolates from another time period over the entire earth and no geographic differentiation is undertaken for the comparison.

As used herein, “conserved” or “conservation” refers to conservation of particular amino acids due to lack of substitution.

As used herein, “Replikin Count” or “Replikin Concentration” refers to the number of Replikin sequences per 100 amino acids in a protein, protein fragment, virus, or organism. A higher Replikin concentration in a first strain of a virus or organism has been found to correlate with more rapid replication of the first virus or organism as compared to a second, earlier-arising or later-arising strain of the virus or organism having a lower Replikin concentration. Replikin concentration is determined by counting the number of Replikin sequences in a given sequence wherein a Replikin sequence is a peptide of 7 to about 50 amino acid residues with a lysine residue on one end and a lysine residue or a histidine residue on the other end wherein the peptide comprises (1) a lysine residue six to ten residues from another lysine residue, (2) a histidine residue, (3) and 6% or more lysine residues, or wherein a Replikin sequence is a nucleic acid that encodes a Replikin peptide sequence.

As used herein, the term “continuous Replikin sequences” means a series of two or more Replikin sequences that are overlapped and/or are directly covalently linked.

### Replikin Cycles in Pathogens

The present invention provides methods of preventing, mitigating, and treating outbreaks of a pathogen by predicting an expansion of a strain of pathogen or an increase in the virulence, morbidity, and/or lethality of a strain of pathogen as compared to another strain of the same pathogen and administering to an animal or patient a compound comprising an isolated or synthesized portion of the structure or genome of the pathogen to mitigate, prevent, or treat the predicted outbreak of the pathogen. The present invention further provides methods of predicting an expanding population of a pathogen or an increase in the virulence, morbidity, and/or mortality of a pathogen comprising identifying a cycle in the Replikin Count in a protein fragment, protein, genome fragment, or genome of a pathogen and predicting an expansion of the population of the pathogen or an increase in the virulence, morbidity, and/or mortality of the pathogen within the identified cycle in Replikin Count.

An increase in the virulence, morbidity, or mortality of a pathogen relative to the virulence, morbidity, and/or mortality of another pathogen of the same species may be predicted by identifying a peak in a cycle or cycles in the concentration of Replikin sequences in the pathogen and predicting an expansion of the population of the pathogen or an increase in the virulence, morbidity, and/or mortality of a pathogen of the same or a related species isolated subsequent to the peak. A Replikin cycle is a cycle in the concentration of Replikin sequences identified in at least four isolates of a species of virus or organism isolated at successive times where (1) the concentration in the first isolate-in-time is higher than the concentration in the second isolate-in-time, the concentration in the third isolate-in-time is higher than the concentration in the second isolate-in-time, and the concentration of the fourth isolate-in-time is lower than the concentration in the third isolate-in-time, or (2) the concentration in the first isolate-in-time is lower than the concentration in the second isolate-in-time, the concentration in the third isolate-in-time is lower than the concentration in the second isolate-in-time, and the concentration of the fourth isolate-in-time is higher than the concentration in the third isolate-in-time. Within a Replikin cycle, an increase in virulence, morbidity, and/or mortality of a pathogen may be predicted for a pathogen arising during a rising portion of the cycle or subsequent to the peak of a cycle. An expanding population may represent an increase in population in a region or expansion from one region into another region. In determining a Replikin cycle, Replikin Counts may represent individual isolates, or mean Replikin Counts of groups of isolates from a given region and/or time period.

In a further non-limiting embodiment, step-wise cycles may be identified between successive time points. In a further embodiment, specific conserved Replikin sequences are identified within the step-wise cycles.

An increase in virulence, morbidity, or mortality of a pathogen may be determined using the methods of the invention in any pathogen or infectious agent where a concentration of Replikins may be determined in the genome, a genome fragment, another nucleic acid sequence, a protein, a protein fragment, or other amino acid sequence from the pathogen. A pathogen may be malaria, West Nile virus, foot and mouth disease virus, porcine circovirus, porcine respiratory and reproductive syndrome virus, taura syndrome virus, white spot syndrome virus, tomato leaf curl virus, bacillus anthracis, small pox virus, human immunodeficiency virus, sindbis virus, hepatitis virus, staphylococcus, legionella, human papilloma virus, Helicobacter, Acetobacter, Aerobacter, Brivebacterium, Clostridium, Erinia, Esheria, Klebsiealla, Maemophilus, Mycoplasma, Psuedomonas, Salmonella, Candida, Entamoeba, or any other form of infectious agent including viruses, bacteria, protozoa, fungi, or other infectious agent.

Any Replikin sequence, Replikin Peak Gene, or protein fragment containing a Replikin sequence or Replikin Peak Gene identified in a strain of pathogen that is predicted to have an increase in virulence, morbidity, or mortality may be isolated and/or synthesized as a diagnostic, therapeutic, or prophylactic agent to mitigate the predicted outbreak of the pathogen.

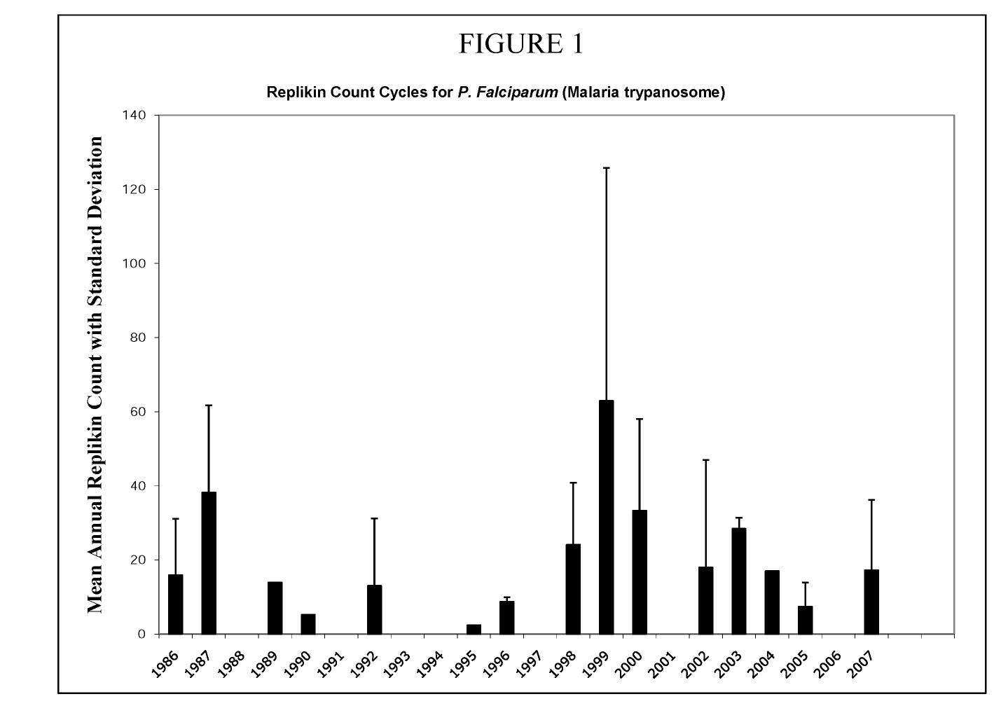

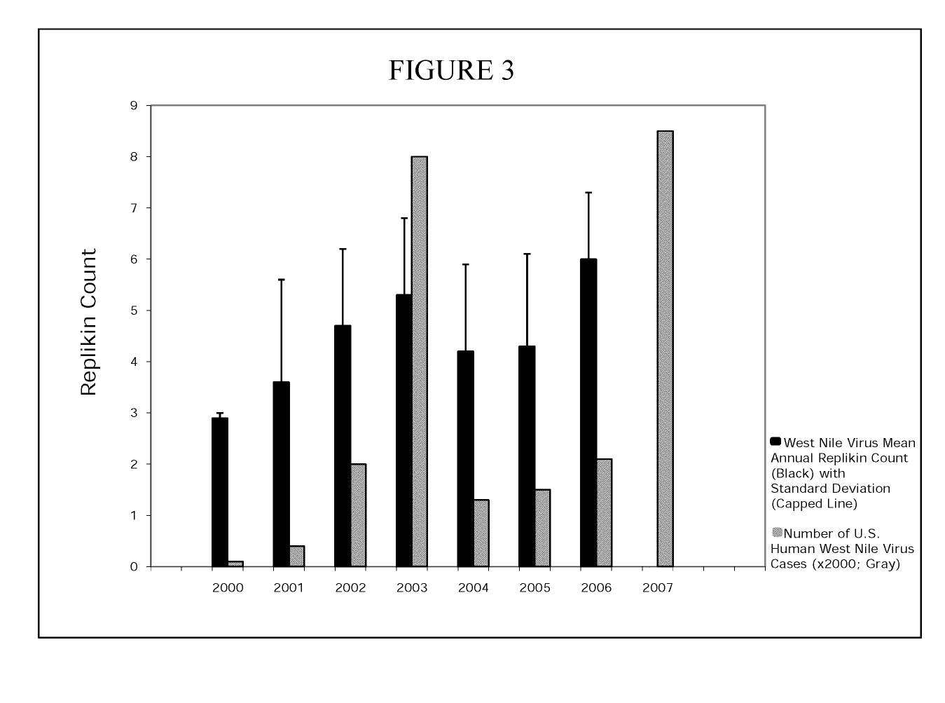

A cycle of Replikin concentration or “Replikin cycle” of a trypanosome may be seen in FIG. 1. Cycles of Replikin concentrations in West Nile virus, foot and mouth disease virus, and influenza virus may be seen in FIGS. 3-6, respectively. A Replikin cycle is identified by initially isolating at least four isolates or groups of isolates from at least four time points or time periods, for example, an isolate or group of isolates may be obtained in 1999, 2001, 2002, and 2004, or may be obtained in January, May, September, and December of a given year. Isolates may be obtained from more than four time points or time periods and precision of a Replikin Cycle generally will improve with increases in the number of isolates per time point or time period and with increases in the number of time points or time periods. The Replikin Count of the genome or expressed proteins of each isolate is determined. Replikin Count may be determined in a Replikin Peak Gene, in the entire genome, in a particular gene or gene segment, or in a particular protein or protein fragment of each of the isolates. Mean Replikin Count for a given time point or given time period is determined if a plurality of isolates has been obtained for the given time point or given time period. Replikin Count may then be analyzed per unit time. A cycle in Replikin concentration is identified by four time points or time periods, where the Replikin Count at a second time point or time period is higher than at first time point or time period, the Replikin Count at a third time point or time period is lower than at second time point or time period, and Replikin Count at a fourth time point or time period is higher than at the third time point or time period; or where the Replikin Count at a second time point or time period is lower than at first time point or time period, the Replikin Count at a third time point or time period is higher than at second time point or time period, and Replikin Count at a fourth time point or time period is lower than at the third time point or time period.

A peak in a Replikin cycle is identified within the cycle at a second time point or time period within a Replikin cycle, wherein the Replikin concentration at a first time point or time period sequentially preceding the second time point or time period is lower than the Replikin concentration at the second time point or time period, and the Replikin concentration at a third time point or time period sequentially following the second time point or time period is lower than the Replikin concentration at the second time point or time period. One of skill in the art will understand that because of the variability of biological systems, a peak may include a general region of a cycle that is generally higher than a sequentially preceding region and generally higher than a sequentially following region rather than an exact time point or time period.

A trough in a Replikin cycle is identified within the cycle is identified within the cycle at a second time point or time period within a Replikin cycle, wherein the Replikin concentration at a first time point or time period sequentially preceding the second time point or time period is higher than the Replikin concentration at the second time point or time period, and the Replikin concentration at a third time point or time period sequentially following the second time point or time period is higher than the Replikin concentration at the second time point or time period. Once again, one of skill in the art will recognize that troughs may be identified as a general region of a cycle that is generally lower than a sequentially preceding region and generally lower than a sequentially following region rather than an exact time point or time period.

Replikin peptides of the invention identified at a peak of the Replikin cycle include Replikin peptides identified at or near the peak of the Replikin cycles including prior to and subsequent to the precise point of the peak. A rising portion of a Replikin cycle is any point at which the trend of Replikin concentration in the Replikin cycle is increasing from at least a first time point or time period to at least a second time point or time period and can include a peak. As may be seen in FIGS. 1-8, an increase in virulence, morbidity, or mortality may be predicted following a rising portion or peak in a Replikin cycle.

In the past, it had been understood that outbreaks of pathogens correlated with increases in Replikin Count and that contractions of pathogenic populations correlated with decreases in Replikin Count. It was not understood, however, that cycles in morbidity, mortality, virulence or population expansion could be directly correlated with cycles in Replikin Count. With the new data presented in the present application, the ordinary skilled artisan will now understand, and it is contemplated by the present invention, that entire Replikin cycles from peak to trough to peak to trough and/or from trough to peak to trough to peak correlate with pathogenic cycles in virulence, morbidity, mortality, and expansion into new regions or hosts. As such, the invention now provides methods of tracking and predicting tracks of pathogens as they increase in virulence, expand in population within a region or into a region, or increase in morbidity or mortality by monitoring changes in Replikin concentration. In the past, it was not possible until months after an event to predict or track the course of pathogens as they increase in virulence, expand in population within region or into a region, or increase in morbidity or mortality where epidemiological data was collected and analyzed post hoc. Replikins analysis provides the skilled artisan with information on population expansion, and increases in virulence, morbidity, and mortality months before or at the very beginning of an outbreak. This information is clearly important for the time needed to organize public health responses, including the testing and administration of specific vaccines. The importance of prior information concerning pathogenic outbreaks may be analogized to the savings of life and property that have resulted from advance warning of hurricanes since information from weather satellites has become available.

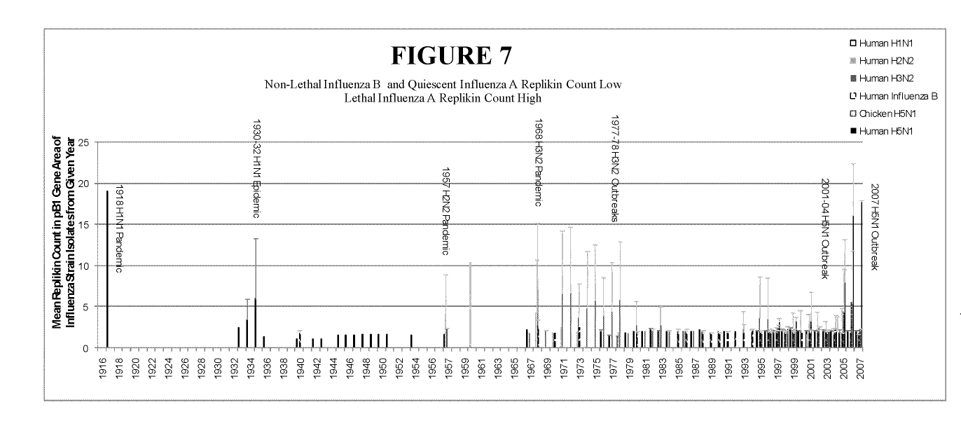

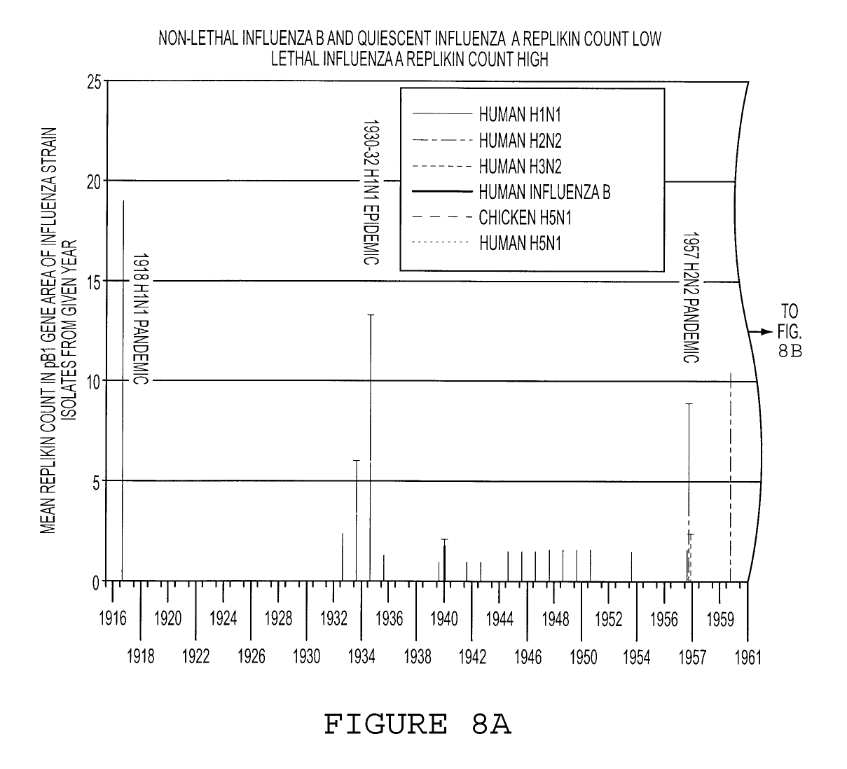

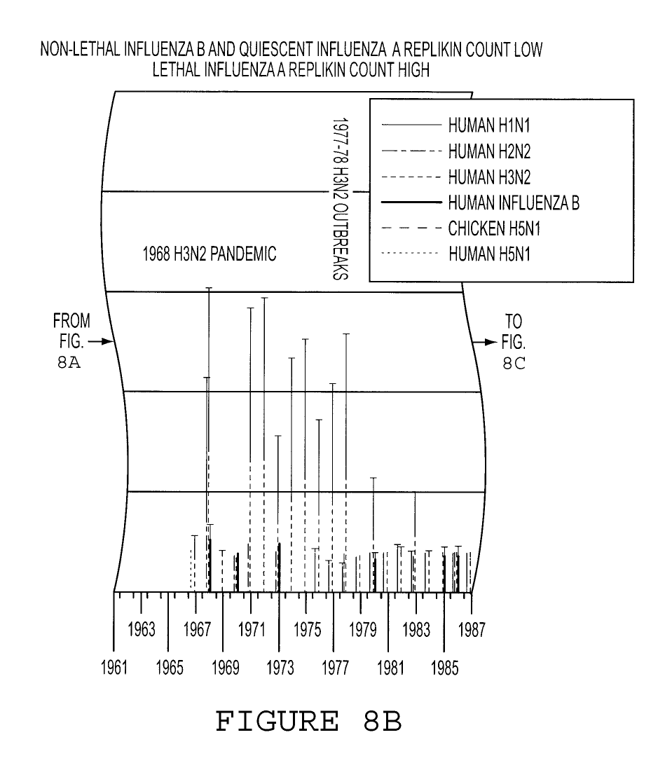

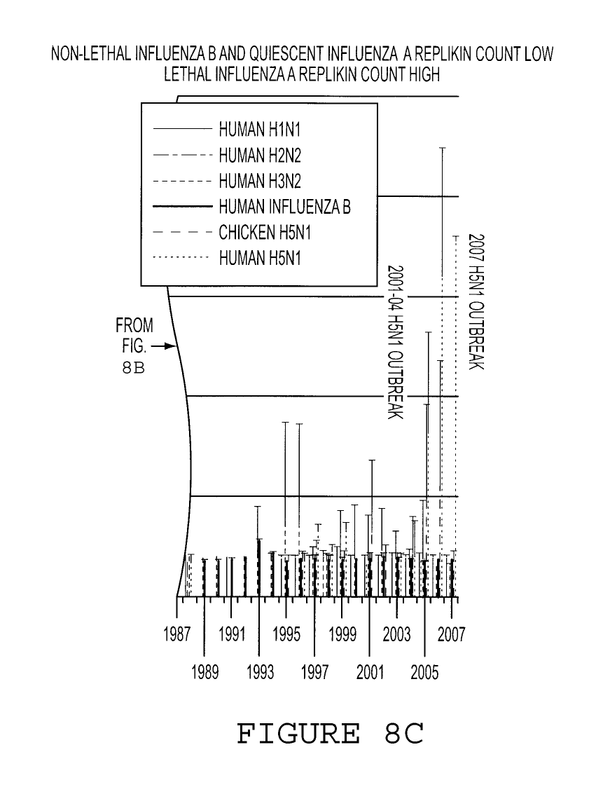

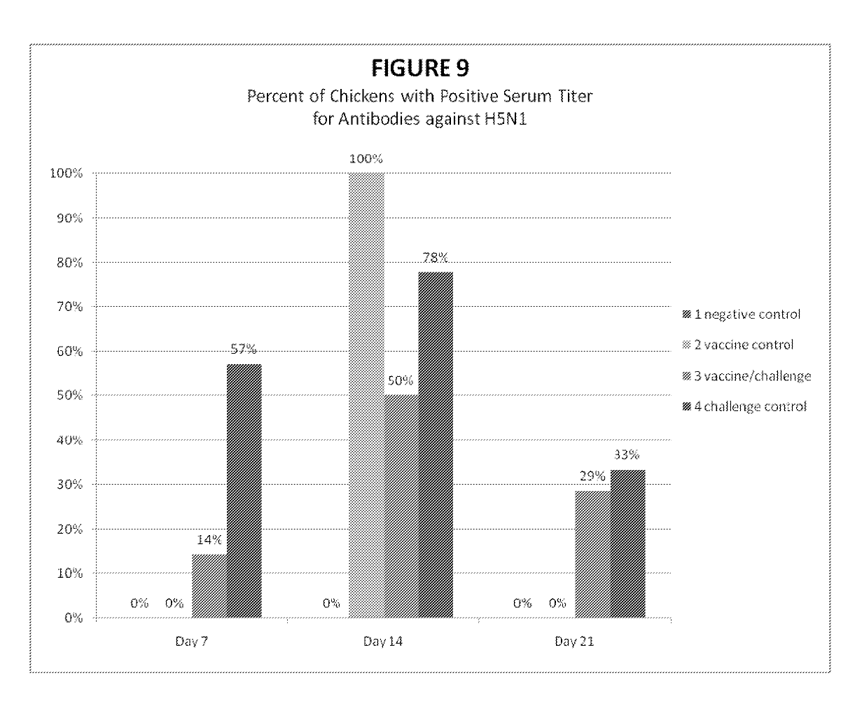

For example, in FIG. 3 the present application provides data demonstrating a cycle of Replikin concentration and a cycle of West Nile virus human morbidity that are observed to correlate. In the past, it was understood that Replikin Count data fluctuate from low to high over time. This may be seen in 20^{th} century data for the H1N1 and H3N2 influenza strains. See FIGS. 7 and 8. But a correlation of cycles from peak to trough and/or from trough to peak was not possible with the earlier data in part because all of the epidemiological data and all of the genomic sequence data of the actual number of cases due to the particular H1N1 strain or H3N2 strain were not available or not recorded. Instead, as may be seen in FIGS. 7 and 8, the only data for H1N1 or H3N2 morbidity in the early- and mid-20^{th} century related to Replikin Count was a record of epidemics or pandemics. As is now shown in FIG. 3 (as well as FIGS. 1, 4, 5, and 6), cycles of Replikin Count correlate with cycles in morbidity over time and, at times, over more than one cycle.

### An Expansion Index for Populations of Pathogens

The present invention also provides a method of predicting an expansion of a strain of pathogen by (1) determining a mean Replikin Count and a standard deviation of the mean Replikin Count for a plurality of isolates of a strain of pathogen for a first time period in a first geographic region; (2) determining a Replikin Count of at least one isolate of the same or a related strain of pathogen from a second time period and/or second geographic region wherein the second time period is different from the first time period and/or the second geographic region is different from the first geographic region; and (3) predicting an expansion of the strain of pathogen isolated in the second time period and/or second geographic region, if the Replikin Count of the at least one isolate is greater than one standard deviation of the mean of the Replikin Count of the plurality of isolates isolated in the first time period and in the first geographic region.

In the above-described method, at least one isolate of the same or related strain of pathogen from a second time period and/or second geographic region may be a plurality of isolates from the second time period and/or second geographic region. In this case, the Replikin Count of each isolate of the plurality of isolates from the second time period and/or second geographic region is compared separately to one standard deviation of the mean. An expansion of pathogen isolated in the second time period and/or second geographic region may also be predicted if the number of Replikin Counts of a plurality of isolates from the second period and/or second geographic region that is greater than one standard deviation of the mean is greater than the number of Replikin Counts of said plurality of isolates from the second period and/or second geographic region that is less than one standard deviation of the mean.

The method may also employ a ratio of the number of Replikin Counts that are greater than one standard deviation of the mean divided by the number of Replikin Counts that are less than one standard deviation of the mean. The ratio is called a Replikin Count Expansion Index (RCE Index). Another way to determine the RCE Index is to divide the percent of Replikin Counts in a plurality of isolates of influenza virus grouped by time and/or region that are higher than one standard deviation of the mean by the percent of Replikin Counts that are lower than one standard deviation of the mean. An RCE Index may be used to quantify the future risk of an outbreak of pathogen by tracking Replikin Counts in strains of pathogen over time.

In determining a RCE Index, the mean Replikin Count of the plurality of isolates from the first time period and geographic region may be considered a control. A control population preferably has a relatively large number of isolates with a relatively small variability in the Replikin Count of the isolates but any population may be deemed a control when a comparison between the control and a related isolate or plurality of isolates is desired. A control may be related to the population that is being studied. For example, if an infection in a bird species, such as swans, is being studied, the control may be something closely related, such as chickens, wherein isolates from chickens may be relatively numerous (if available) and relatively stable (if possible) wherein stability in Replikin Count through the population demonstrates a level of equilibrium between the expansion and contraction of the strain or a related strain of influenza virus in chickens. A control may reflect a highest number of isolates reported in a year or in several years in a geographic area.

An expansion of a strain of pathogen may be determined using the methods of the invention in any pathogen or infectious agent where a concentration of Replikins may be determined in the genome, a genome fragment, another nucleic acid sequence, a protein, a protein fragment, or other amino acid sequence from the pathogen. A pathogen may be malaria, West Nile virus, foot and mouth disease virus, influenza virus, porcine circovirus, porcine respiratory and reproductive syndrome virus, taura syndrome virus, white spot syndrome virus, tomato leaf curl virus, bacillus anthracis, small pox virus, human immunodeficiency virus, sindbis virus, hepatitis virus, staphylococcus, legionella, or any other form of infectious agent including viruses, bacteria, protozoa, fungi, or other infectious agent.

Any Replikin sequence, Replikin Peak Gene, or protein fragment containing a Replikin sequence or Replikin Peak Gene identified in a strain of pathogen that is predicted to have an increase in virulence, morbidity, or mortality may be isolated and/or synthesized as a diagnostic, therapeutic, or prophylactic agent to mitigate the predicted outbreak of the pathogen.

### Diagnostics and Therapies Using Replikin Peptides Identified in Replikin Cycles

The present invention further provides the opportunity to identify Replikin sequences (including nucleic acid sequences and peptide sequences) for diagnostic, therapeutic, or preventive purposes (such as the construction of vaccines and other pharmaceuticals). The present invention contemplates, for example, Replikin peptides identified within a pathogen where the pathogen is predicted to have an expanding population or a higher virulence, morbidity, and/or mortality than another pathogen of the same or a related species based on the predictive methods of the invention. Replikin peptides identified in an isolate of a pathogen, wherein said isolate is isolated during a rising portion of a cycle in Replikin concentration among a plurality of isolates of the pathogen or is isolated at a peak in a cycle in Replikin concentration among a plurality of isolates of the pathogen, are useful for diagnostic, therapeutic, and preventive purposes. For example, a Replikin peptide identified in the genome of an isolate identified in a rising portion of a cycle in Replikin concentration or identified at a peak in a cycle in Replikin concentration is useful as a peptide to stimulate the immune system of a human or animal to produce an immune response against infection by the pathogen or to produce antibodies against a pathogen predicted to have higher virulence, morbidity, and/or mortality. One of ordinary skill in the art will recognize that antibodies against these pathogens are useful for diagnosing the more highly virulent or mortal disease in a subject or useful as therapies against the infection either as a prophylactic or after onset of the infection.

Additionally, Replikin peptides identified during a rising portion in Replikin concentration in a Replikin cycle or identified at or near a peak in Replikin concentration in a Replikin cycle that are conserved during the rising portion of the Replikin cycle are useful as compounds for diagnostic, therapeutic, and preventive purposes. Conservation of the Replikin peptides during a rise in virulence, morbidity, and/or mortality provides targets that are more constant and likely more involved in the mechanisms of rapid replication that provide the predicted increase in virulence, morbidity, and/or mortality. As such, these conserved Replikin peptides are of use as compounds or in compositions for stimulating the immune system of a subject to produce an immune response, an antibody response, and/or a protective effect in the subject.

Replikin peptides identified and isolated using the methods of the invention include influenza peptides such as HAQDILEKEHNGKLCSLKGVRPLILK (SEQ ID NO: 1), KEHNGKLCSLKGVRPLILK (SEQ ID NO: 2), KKNNAYPTIKRTYNNTNVEDLLIIWGIHH (SEQ ID NO: 3), HHSNEQGSGYAADKESTQKAIDGITNK (SEQ ID NO: 4), HDSNVKNLYDKVRLQLRDNAK (SEQ ID NO: 5), KVRLQLRDNAKELGNGCFEFYH (SEQ ID NO: 6), KDVMESMDKEEMEITTH (SEQ ID NO: 7), HFQRKRRVRDNMTKK (SEQ ID NO: 8), KKWSHKRTIGKKKQRLNK (SEQ ID NO: 9), HKRTIGKKKQRLNK (SEQ ID NO: 10), HEGIQAGVDRFYRTCKLVGINMSKKK (SEQ ID NO: 11); or HSWIPKRNRSILNTSQRGILEDEQMYQKCCNLFEK (SEQ ID NO: 12), West Nile virus peptides such as KIIQKAHK (SEQ ID NO: 13), HLKCRVKMEK (SEQ ID NO: 14), KLTSGHLK (SEQ ID NO: 15), or HNDKRADPAFVCK (SEQ ID NO: 16), and foot and mouth disease peptides such as HKQKIIAPAK (SEQ ID NO: 17) and HKQKIVAPVK (SEQ ID NO: 18).

Identification of portions of a pathogen (such as Replikin Peak Genes or Replikin peptides) predicted to expand in population provide unique compounds for diagnostics and treatment of expanding pathogens, wherein the unique compounds would otherwise not be identifiable but for the methods of the invention and the compounds disclosed herein.

The invention further contemplates use of the Replikin peptide as immunogenic compositions and contemplates the immunogenic compositions as vaccines, including vaccines that provide an immune response, vaccines that provide a humoral immune response, vaccines that provide an antigenic immune response, and vaccines that provide a protective effect. The invention additionally contemplates an antibody to the Replikin peptides of the invention.

High Replikin Counts and RPGs have been shown to be related to rapid replication, viral outbreaks, epidemics, morbidity and host mortality, for example, in influenza virus strains, including H5N1, in SARS coronavirus, in shrimp taura syndrome virus, and in foot and mouth disease virus. Replikin sequences identified at or near the peak of the Replikin cycle or during a rising portion of the Replikin cycle in a pathogen are appropriate peptides for diagnostics, vaccines, and other treatments.

Because Replikin sequences are chemically defined, the sequences may be synthesized by organic chemistry rather than biological techniques, and thus are potentially more specific, more reproducible and more reliable. The chemically defined Replikin sequences identified by Applicants are likewise potentially freer from adverse reactions that are characteristic of biologically derived vaccines and antibodies.

Mitigating and Treating Outbreaks of Pathogen with Cyclic Replikin Counts

One aspect of the present invention provides methods of preventing, mitigating, or treating pathogenic outbreaks predicted through analysis of cycles of Replikin Counts or through analysis of controls using mean Replikin Counts and standard deviation (e.g., Replikin Count Expansion Index). For example, advance information concerning Replikin peptides and Replikin Peak Genes in expanding strains of a pathogen allows for the rapid production of specific effective synthetic vaccines using one, or a combination, of Replikin peptides or using Replikin Peak Genes. Such synthetic vaccines have been demonstrated in rabbits, chickens, and shrimp. See, e.g., Examples 6 and 7 of U.S. application Ser. No. 11/355,120, filed Feb. 16, 2006 and Example 2 of U.S. application Ser. No. 12/108,458, filed Apr. 23, 2008. For example, a mixture of Replikin peptides administered orally to shrimp provided up to a 91% protective effect for shrimp challenged with taura syndrome virus. Taura syndrome virus is an often lethal rapidly-replicating pathogen that has a significant negative impact on the shrimp industry.

Synthetic Replikin vaccines have also been demonstrated in the H5N1 strain of influenza virus in chickens. For example, in a test of chickens administered a mixture of twelve H5N1 Replikin peptides from the hemagglutinin and pB1 gene areas intranasally, intraocularly, and by spray inhalation and challenged with low pathogenic H5N1 influenza isolated from a black duck in the state of North Carolina in the United States, a protective effect was observed at both the entry site of influenza (diminished antibody production in the mucus was observed as compared to a control) and at excretion sites of influenza (influenza virus was not observed excreted in feces or saliva from treated chickens as compared to a control). See Example 10 below.

Administration of Replikin peptides in both shrimp and chickens appears to have provided a notable measure of mucosal immunity. For example, in Example 2 of U.S. application Ser. No. 12/108,458, a mixture of Replikin peptides was administered by mouth to shrimp later challenged with taura syndrome virus. The 91% protective effect of the vaccine is expected to have been a result, at least in part, of a mucosal immune-like responses in the gut of the shrimp.

Likewise, in chickens, the administration of a mixture of Replikin peptides provided a protective effect against entry of the H5N1 virus. For example, as may be seen in Example 10 below, three of six vaccinated chickens, when inoculated with H5N1 virus, produced no measurable amount of antibodies against H5N1 in their serum. Instead, the virus was apparently blocked by mucosal immunity from even entering the chickens' blood stream. For those three chickens in which a serum immune response was measured (that is, virus entered the host and was presented to antibody generating cells), the vaccine additionally provided a protective effect against replication of the virus in the chickens' system (no virus was excreted in the feces or saliva of the chickens). As such, mucosal immunity, in addition to other immunities, is an important aspect of the immunity imparted by Replikin-based vaccines.

Cyclic increases in Replikin concentration in the genome can be a mechanism of expansion of a pathogen into a territory. The Replikin concentration in each Replikin Peak Gene of each Replikin cycle in an expanding population apparently may build on the previous one. Timely, repeated analyses of cyclic changes in a virus' Replikin structure is useful to bring current the targets for the chemical synthesis of Replikin vaccines having a best fit for emerging pathogens having increased virulence, morbidity, and/or lethality. These strain-specific vaccines may be manufactured in seven days as have been demonstrated with a 91% protection of shrimp against the lethal taura syndrome virus. See, e.g., U.S. application Ser. No. 12/108,458, filed Apr. 23, 2008 (incorporated herein in its entirety by reference).

### Replikin Cycles in Malaria

The present invention provides methods of predicting an expansion of the population of a trypanosome that causes malaria or an increase in the virulence, morbidity, and/or mortality of a trypanosome that causes malaria as compared to another trypanosome of the same species or a related species. An expanding population or increase in virulence, morbidity, and/or mortality of a trypanosome that causes malaria may be predicted by identifying a cycle of Replikin concentration among a plurality of isolates of the species of trypanosome and identifying a rising portion or peak in that cycle. An increase in virulence, morbidity, and/or mortality is predicted following the time point or time period when the rising portion or peak is identified. An expanding population may represent an increase in population in a region or expansion from one region into another region.

A further non-limiting embodiment of one aspect of the invention provides a method of predicting an increase in morbidity and mortality in malaria comprising: (1) determining the mean Replikin Count in a plurality of isolates of a malarial trypanosome at a plurality of successive time points; (2) comparing the mean Replikin Count at least four successive time points and identifying at least one cycle of increasing mean Replikin Counts over the at least four time points; and (3) predicting an increase in morbidity and/or mortality following in time the increase in mean Replikin count in at least one of said cycles. In a further non-limiting embodiment, step-wise cycles are identified between successive time points. In a further non-limiting embodiment, specific conserved Replikin sequences are identified within the step-wise cycles. In a further non-limiting embodiment, Replikin sequences are identified at the peak of a step-wise cycle. The Replikin sequences identified at the peak of a step-wise cycle are useful for developing a vaccine or therapeutic composition of an isolated or synthesized Replikin peptide for use in preventing or treating outbreaks of malaria with relatively higher mortality.

FIG. 1 illustrates cycling between 1986 and 2007 of annual mean Replikin concentration in the histidine rich protein of Plasmodium falciparum. P. falciparum is a trypanosome that is most commonly associated with malaria. Cycles are observable with peaks in 1987 and 1999. A new cycle appears to have begun between 2005 and 2007. Publicly available accession numbers at www.pubmed.com containing amino acid sequence listings for P. falciparum were queried using the automated FluForecast® software (Replikins, Ltd., Boston, Mass.). The software analyzed the Replikin Count of each available sequence between 1986 and 2007. The area of the P. falciparum genome observed to have the highest concentration of continuous Replikin sequences per 100 amino acids was found to be the histidine rich protein. The histidine rich protein includes the knob-associated histidine rich protein.

Analysis of the mean annual Replikin Count of the histidine rich protein between 1986 and 2007 revealed cycles of Replikin Count. A first rising portion followed by a decreasing portion of the cycle was observed from 1986 to 1995. A second rising portion followed by a decreasing portion was observed from 1996 to 2005. A first peak was identified in 1987 with a mean annual Replikin Count of 38.2 and standard deviation of ±23.5. A second peak was identified in 1999 with an even higher mean annual Replikin Count of 62.9 and standard deviation of ±62.9 (overlap of Replikin sequences within an amino acid sequence generates a Replikin Count of greater than 100 Replikin sequences per 100 amino acids in some sequences). Both the 1987 peak and the 1999 peak were observed to be related to higher human mortality. Following the 1999 peak, mean annual Replikin Counts were observed to fall to a low of 7.4 in 2005 with a standard deviation of ±6.5. Mortality rates likewise fell between 2000 and 2005. A third malaria Replikin cycle appears to have begun in 2005 with the observed mean annual Replikin Count increasing from 7.4±6.5 in 2005 to 17.2±19 in 2007. The beginning of a new cycle provides a prediction that Replikin Count may continue to increase along with an increase in malaria mortality rate.

The cycling observable in FIG. 1 has also been observed in viruses, namely, the H1N1, H2N2, H3N3, H5N1, H3N8, and H9N2 strains of influenza virus, in West Nile virus, and in foot and mouth disease virus. See FIGS. 1-8. Thus Replikin cycles are observable in both viruses and organisms. Similar correlations between Replikin Count and mortality have also been shown in an influenza H5N1 cycle between 1997 and 2007. See, e.g., U.S. application Ser. No. 12/010,027, filed Jan. 18, 2008 (FIG. 8).

The data for FIG. 1 are seen in Table 1 below. Mean annual Replikin Count, standard deviation, significance of annual mean Replikin Count to the lowest annual mean Replikin Count and to the previous annual mean Replikin Count, and number of accession numbers analyzed per annum is provided.

TABLE 1
P. falciparum Replikin Count

| | | | | | Number of |
|---|---|---|---|---|---|
| | | | | | Accession |
| | | | | | Records |
| | Mean | | Significance | Significance | for |
| | Replikin | Standard | (compared to | (compared to | malaria |
| Year | Count | Deviation | lowest value) | previous year) | isolates |
| 1986 | 15.9 | 15.2 | low > 0.5 | | 6 |
| 1987 | 38.2 | 23.5 | low < 0.005 | <0.02 | 11 |
| 1988 | | | | | |
| 1989 | 13.9 | 0 | | <0.005 | 1 |
| 1990 | 5.2 | 0 | | | 1 |
| 1991 | | | | | |
| 1992 | 13 | 18.2 | >0.5 | <0.2 | 9 |
| 1993 | | | | | |
| 1994 | | | | | |
| 1995 | 2.4 | 0 | | <0.1 | 1 |
| 1996 | 8.7 | 1.2 | <0.01 | <0.01 | 3 |
| 1997 | | | | | |
| 1998 | 24.1 | 16.7 | <0.01 | <0.04 | 7 |
| 1999 | 62.9 | 62.9 | <0.2 | <0.24 | 4 |
| 2000 | 33.3 | 24.7 | <0.3 | <0.4 | 3 |
| 2001 | | | | | |
| 2002 | 18 | 29 | >0.5 | <0.3 | 13 |
| 2003 | 28.4 | 3 | <0.001 | <0.2 | 7 |
| 2004 | 17 | 0 | <0.001 | | 1 |
| 2005 | 7.4 | 6.5 | <0.05 | <0.02 | 5 |
| 2006 | | | | | |
| 2007 | 17.2 | 19 | >0.5 | <0.2 | 8 |

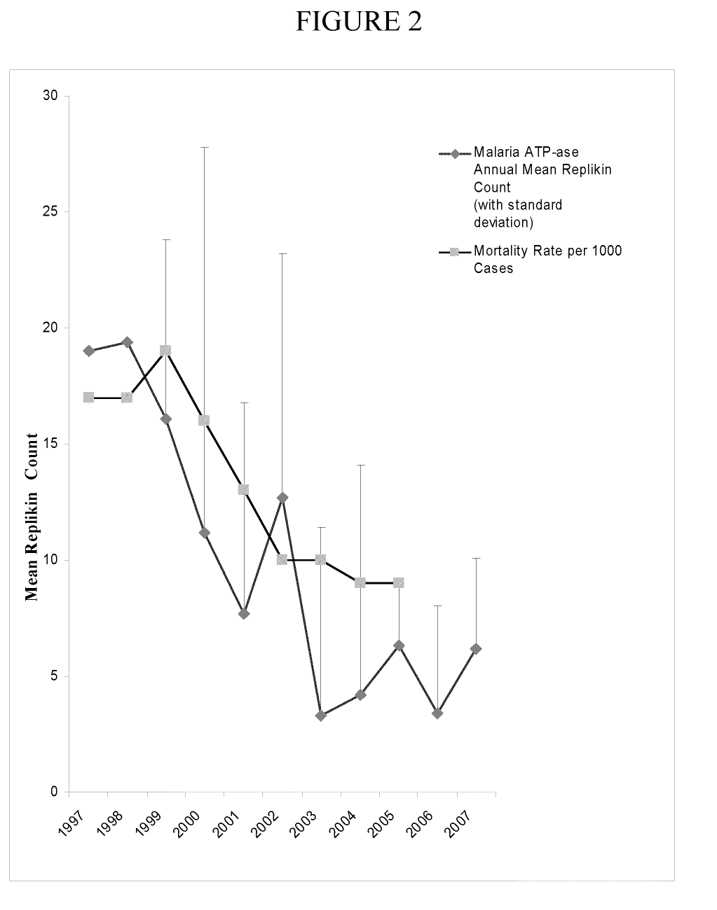

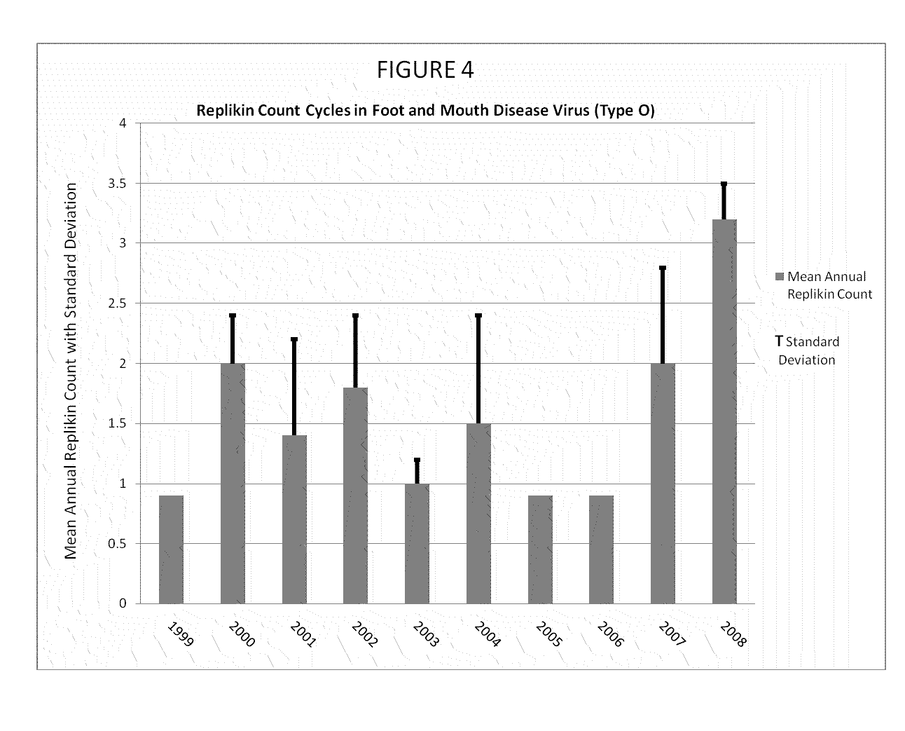

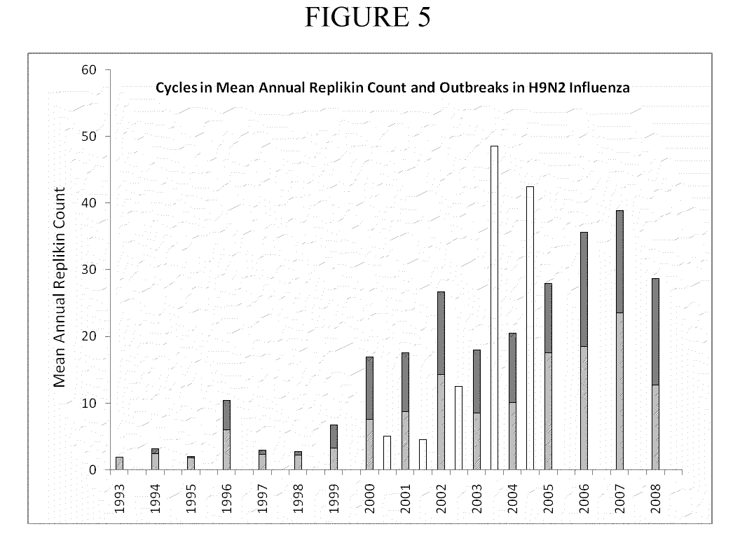

As is seen in FIG. 1 and Table 1 above and as also seen in FIG. 2 and Table 6 below, changes in malaria virulence and mortality may be predicted by identifying a peak within an identified cycle in the Replikin concentration of isolates of a plurality of the trypanosome and predicting an increase in the virulence, morbidity, and/or mortality of a trypanosome of the same species isolated at a time point or time period subsequent to the time point or time period of the identified peak in the cycle of Replikin concentration. In contrast to FIGS. 3, 7, and 8 for West Nile virus and influenza, morbidity data is not reflected in the analysis of malaria in FIG. 1 and is also not contained in FIG. 2, which compares Replikin Count in the ATP-ase protein of P. falciparum to mortality. Use of mortality data and not morbidity data in FIGS. 1 and 2 and their related analysis and tables is based on the skilled artisan's understanding that morbidity data in malaria is generally unreliable while mortality data is considered more reliable. While the analysis of FIG. 1 and the data in FIG. 2 demonstrate a relationship between Replikin Count in P. falciparum and mortality, the skilled artisan will understand that the relationship would also be expected to extend to morbidity and general virulence in malaria just as it has in West Nile virus (see FIG. 3), foot and mouth disease (see FIG. 4), and influenza (see FIGS. 5-8).

Cyclic increases in Replikin concentration in the genome can be a mechanism of expansion of an infectious organism into a territory. The Replikin concentration in each Replikin Peak Gene of each Replikin cycle apparently builds on the previous one. In both the mosquito-borne West Nile Virus and mosquito-borne malaria trypanosomes, this build-up probably occurs during winter seasons, dry seasons, or otherwise dormant periods. Timely, repeated analyses of cyclic changes in the organism's Replikin structure is useful to bring current the targets for the chemical synthesis of Replikin vaccines having a best fit for emerging pathogens having increased virulence, morbidity, and/or mortality. These strain-specific vaccines may be manufactured in seven days as has been demonstrate with a 91% protection of shrimp against the lethal taura syndrome virus. See, e.g., U.S. application Ser. No. 12/108,458, filed Apr. 23, 2008 (incorporated herein in its entirety by reference).

The Replikin cycle may be identified in any trypanosome that causes malaria. For example, it may be identified in the genome of a trypanosome, including P. falciparum, Plasmodium vivax, Plasmodium ovale, or Plasmodium malariae. The Replikin cycle may likewise be identified in the histidine rich protein or in the ATP-ase protein, including in these proteins in P. falciparum. The Replikin cycle may likewise be identified in a Replikin Peak Gene of a trypanosome that causes malaria.

Malaria trypanosomes have been found to have the highest Replikin counts seen to date in any infectious organisms—up to twenty times those in influenza and West Nile Virus. Consistent with these high counts, trypanosomes have one of the highest replication rates in nature. This property may account in part for the resistance of malaria to previous attempts at vaccination. The discovery of the relation of Replikin sequences to rapid replication offers a new approach, and means, to inhibit rapid replication in malaria.

In the data analysis reported in Tables 1 and 5, as well as FIG. 1, Replikin sequences were identified as conserved sequences in the histidine-rich protein of malaria in the rising portion and peak of the illustrated Replikin cycle. Such sequences are useful as diagnostic and therapeutic compounds for virulent malaria infections. The sequences are useful in the production of immunogenic compounds including vaccines and may be comprised in immunogenic therapies including vaccines.

For example, Replikin peptides identified in the ABU43157 isolate in 2007 are available as a diagnostic, therapeutic, or preventive compounds or compositions of the invention because they were identified in a rising portion of a Replikin cycle. See FIG. 1 and Table 5. Replikin peptides identified in the 1999 isolate at accession number CAD49281 are likewise Replikin peptides of the invention. The 1999 isolate is present at the peak of a Replikin cycle, as such, Replikin peptides identified in the isolate reported at CAD49281 may be used as immunogenic compounds. Additionally, the 1998 accession number XP001349534 is identified as from an isolate from a rising portion in a Replikin cycle. Replikin peptides identified in XP001349534 are likewise useful as immunogenic compounds or vaccines or for diagnosis or treatment of malaria. See FIG. 1 and Table 5 for all accession numbers discussed in this paragraph.

### Replikin Count Cycles in West Nile Virus

In a further aspect of the invention, an expanding population of West Nile virus or an increase in virulence, morbidity, or morality of West Nile virus may be predicted by identifying a cycle of Replikin concentration in isolates of West Nile virus and predicting an expanding population of virus or an increase in virulence, morbidity, and/or mortality of West Nile virus following a rising portion, or peak in the cycle of Replikin concentration. An expanding population may represent an increase in population in a region or expansion from one region into another region.

For example, using analysis of Replikin sequences in West Nile virus, including, for example, analysis of Replikin sequences in the envelope protein of West Nile virus, a correlation between virus biochemical cycles and virus virulence, morbidity, and/or mortality cycles may be identified and used to predict expansions in a virus population or increases in virulence, morbidity, and/or mortality in a virus in a host population. A non-limiting embodiment of the aspect of the invention provides a method of predicting an increase in morbidity in a viral disease such as West Nile virus comprising: (1) determining the mean Replikin Count in genomes of a plurality of isolates of a virus at a plurality of successive time points; (2) comparing the mean Replikin Count at least four successive time points and identifying at least two peaks or two troughs in the trend of Replikin Counts over the at least four time points; and (3) predicting an increase in morbidity following in time the increase in mean Replikin count within said cycles. In a further non-limiting embodiment, step-wise cycles are identified between successive time points. In a further embodiment, specific conserved Replikin sequences are identified within the step-wise cycles.

Table 2, below, provides data from analysis of envelope protein sequences in West Nile virus available at www.pubmed.com for isolates from 2000 through 2007. The data, which are illustrated in FIG. 3, provide an example of cycling in mean annual Replikin Count in a virus wherein the cycle predicts morbidity. The data additionally further support immunogenic compounds, diagnostic compounds, and, among other things, vaccines because they support the principles upon which such Replikin vaccines and other therapies are based including, in particular, the role Replikin sequences play in virulence and morbidity in pathogenic diseases, the correlation of Replikin Count with pathogenicity generally, and targeting of the Replikin structures for control of rapid replication and disease generally. See, e.g., U.S. application Ser. No. 11/355,120, filed Feb. 16, 2006 and U.S. application Ser. No. 12/010,027, filed Jan. 18, 2008 (each incorporated herein by reference in their entirety).

TABLE 2
Mean Annual Replikin Count in West Nile Virus Envelope Protein

| | | Mean | | |
|---|---|---|---|---|
| | No. of | Replikin | | |
| | Isolates per | Count per | | |
| Year | year | year | S.D. | Significance |
| 2000 | 4 | 2.9 | 0.1 | low p < 0.001, prev |
| | | | | p < 0.001 |
| 2001 | 130 | 3.6 | 2.0 | low p < 0.02, prev |
| | | | | p < 0.001 |
| 2002 | 18 | 4.7 | 1.5 | low p < 0.001, prev |
| | | | | p < 0.005 |
| 2003 | 94 | 5.3 | 1.5 | low p < 0.001, prev |
| | | | | p < 0.05 |
| 2004 | 55 | 4.2 | 1.7 | low p < 0.001, prev |
| | | | | p < 0.001 |
| 2005 | 125 | 4.3 | 1.8 | low p < 0.001, prev |
| | | | | p > 0.50 |
| 2006 | 312 | 6.0 | 1.3 | low p < 0.001, prev |
| | | | | p < 0.001 |
| 2007 | (Incomplete) | (Incomplete) | (Incomplete) | (Incomplete) low |
| | 27 | 4.6 | 1.2 | p < 0.001, |
| | | | | prev p < 0.001 |
| 2008 | (Incomplete) | (Incomplete) | (Incomplete) | (Incomplete) low |
| | 5 | 5.5 | 0.7 | p < 0.002, |
| | | | | prev p < 0.04 |

In FIG. 3 and Table 2, cycles in mean Replikin Count in isolates of West Nile virus are detectable because of repeating conserved virus structures and continuity of the Replikin phenomenon through time. The identified cycles provide a novel method of (1) determining the growth, spread, and path of an emerging disease, (2) predicting and tracking the occurrence and intensity of viral and other organism outbreaks by tracking changes in Replikin Count manually or using computer programs such as ReplikinsForecast™ (available through Replikins LLC, Boston, Mass.) (see, e.g., U.S. application Ser. No. 11/116,203, filed Apr. 28, 2005, which is incorporated herein in its entirety by reference), (3) designing and chemically synthesizing vaccines that contain both older conserved Replikins as well as newer ones to provide the most accurate and maximal anti-organism immune stimulating properties, (4) designing and chemically synthesizing antibodies that contain reactive sites against both older conserved Replikins and newer ones, to provide the most accurate and maximal anti-organism immune protective properties, and (5) designing and chemically synthesizing compounds that contain reactive sites against both older conserved Replikins and newer ones, to provide the most accurate and maximal anti-organism protective properties.

Immunogenic compounds for therapeutic vaccines against West Nile virus include, for example, KIIQKAHK (SEQ ID NO: 13), HLKCRVKMEK (SEQ ID NO: 14), KLTSGHLK (SEQ ID NO: 15), and HNDKRADPAFVCK (SEQ ID NO: 16). These Replikin peptide sequences are conserved within the step-wise cycles of West Nile virus in FIG. 3, which render them of particular use for therapies against expanding West Nile virus populations following the cyclic peaks identified in FIG. 3. The sequences may be administered to animals or humans as a vaccine. A vaccine may comprise a pharmaceutically acceptable carrier and/or adjuvant. A vaccine can be manufactured within seven days of the identification of sequences, such as these, that are conserved in step-wise cycles identified in the Replikin Count of a pathogen such as West Nile virus. The sequences may likewise be used for diagnostic purposes to identify isolates of the expanding population of West Nile virus.

### Replikin Count Cycles in Foot and Mouth Disease Virus

In a further aspect of the invention, an expanding population of foot and mouth disease virus or an increase in virulence, morbidity, or mortality of West Nile virus may be predicted by identifying a cycle of Replikin concentration in isolates of foot and mouth disease virus and predicting an expanding population of virus or an increase in virulence, morbidity, and/or mortality of virus following a rising portion, or peak in the cycle of Replikin concentration. An expanding population may represent an increase in population in a region or expansion from one region into another region.

For example, using analysis of Replikin sequences in foot and mouth disease virus, including, for example, analysis of Replikin sequences in the VP1 protein of foot and mouth disease virus, a correlation between virus biochemical cycles and virus virulence, morbidity, and/or mortality cycles may be identified and used to predict expansions in a virus population or increases in virulence, morbidity, and/or mortality in a virus in a host population. A non-limiting embodiment of the aspect of the invention provides a method of predicting an increase in morbidity in a viral disease such as foot and mouth disease virus comprising: (1) determining the mean Replikin Count in genomes of a plurality of isolates of a virus at a plurality of successive time points; (2) comparing the mean Replikin Count at least four successive time points and identifying at least two peaks or two troughs in the trend of mean Replikin Counts over the at least four time points; and (4) predicting an increase in virulence and/or morbidity following in time an increase in mean Replikin count within a cycle. In a further non-limiting embodiment, step-wise cycles are identified between successive time points. In a further embodiment, specific conserved Replikin sequences are identified within the step-wise cycles.

Increased Replikin Counts provide advance warnings of Foot and Mouth Disease outbreaks and the basis of a conserved synthetic FMDV Vaccine. One aspect of the invention contemplates provision of advance warning of outbreaks of FMDV by identifying cycles in the Replikin Count of isolates of FMDV over time. As may be seen from the data in FIG. 4, in 2000, an outbreak of Foot and Mouth Disease Virus (Type O) (FMDV) was predicted by a peak in annual mean Replikin Count. An outbreak in 2001-2002 was observed in the United Kingdom and in the Netherlands. In a new cycle beginning in 2005, the highest Replikin counts in ten years, observed in 2007 and 2008, were followed by severe FMDV outbreaks in 2008 and 2009 in the Middle East, Africa, India, China, and other Asian countries. Replikin peptide structures found to be conserved over decades are now the basis of a synthetic Replikins vaccine for FMDV. Replikin sequences identified as conserved within the Replikin cycles in FIG. 4 include HKQKIIAPAK (SEQ ID NO: 17) and HKQKIVAPVK (SEQ ID NO: 18). These sequences are also observed to be conserved over time in isolates of foot and mouth disease type A.

FIG. 4 illustrates cycles of Replikin Count in Type O isolates of FMDV. The data illustrated in FIG. 4 are contained in Table 3.

TABLE 3
Foot and Mouth Disease Virus Protein Replikin Cycles

| | Mean Replikin Count of | |
|---|---|---|
| | Reported FMDV Isolates in | Standard |
| Year | Listed Year | Deviation |
| 1999 | 0.9 | 0 |
| 2000 | 2 | 0.4 |
| 2001 | 1.4 | 0.8 |
| 2002 | 1.8 | 0.6 |
| 2003 | 1 | 0.2 |
| 2004 | 1.5 | 0.9 |
| 2005 | 0.9 | 0 |
| 2006 | 0.9 | 0 |
| 2007 | 2 | 0.8 |
| 2008 | 3.2 | 0.3 |

The data in Table 3 and FIG. 4 illustrate that the annual Replikin Counts (Mean and Standard Deviation (SD)) in Foot and Mouth disease virus occurred in two rising portions and a decreasing portion. The first rising portion followed by the first decreasing portion occurs from 1999-2005 and the second rising portion occurs from 2005-2008. Increases in Replikin Counts provided advance warning signals (p<<0.001) prior to severe FMDV outbreaks in the U.K. and the Netherlands in 2001-2002, and in the Middle East, Africa, India, and Asia in 2008-2009.

Replikin peptides (1) were identified and counted automatically, with tests of statistical significance of changes, using a software program (ReplikinsForecast™ Replikins LLC, Boston, Mass.) designed to analyze the protein sequences of any organism, in this case FMDV published in PubMed. When the history of each Replikin structure in the virus was tracked for its occurrence in each virus specimen in each of the years for which virus sequence data was published, conservation of Replikin structures for decades was found. The structure of these conserved Replikins is the basis of synthetic Replikins vaccines for FMDV.

Replikin peptides conserved in FMDV over decades include HKQKIIAPAK (SEQ ID NO: 17) and HKQKIVAPVK (SEQ ID NO: 18). Sequences identified within Replikin cycles and as conserved within Replikin cycles are particularly useful for diagnostic and therapeutic purposes. For example, the sequences identified as new and/or conserved in FMDV Replikin cycles are useful for (1) designing and chemically synthesizing vaccines that contain both older conserved Replikins as well as newer ones to provide the most accurate and maximal anti-organism immune stimulating properties, (2) designing and chemically synthesizing antibodies that contain reactive sites against both older conserved Replikins and newer ones to provide the most accurate and maximal anti-organism immune protective properties, and (3) designing and chemically synthesizing compounds that contain reactive sites against both older conserved Replikins and newer ones to provide the most accurate and maximal anti-organism protective properties.

### Predicting Expansion of Populations of Influenza Virus

One aspect of the present invention provides methods of predicting an outbreak of influenza by predicting an increase in the virulence, morbidity, and/or lethality of a strain of influenza virus or an expansion of the population of a strain of influenza virus using a Replikin Count Virus Expansion Index. In this aspect of the invention, an increase in virulence, morbidity, and/or lethality or an expansion of a strain of influenza virus is predicted by (1) determining a mean Replikin Count and a standard deviation from the mean Replikin Count for a plurality of isolates of a strain of influenza virus for a first time period in a first geographic region, (2) determining a Replikin Count of at least one isolate of the same or a related strain of influenza virus from a second time period and/or a second geographic region different from the first time period and/or the second geographic region, and (3) predicting an increase in virulence, morbidity, and/or lethality or an expansion of the strain of influenza isolated in the second time period and/or second geographic region, if the Replikin Count of the at least one isolate from a second time period and/or a second geographic region is greater than one standard deviation of the mean of the Replikin Count of the plurality of isolates isolated in the first time period in the first geographic region.

In the above-described method, at least one isolate of the same or related strain of influenza virus from a second time period and/or second geographic region may be a plurality of isolates from the second time period and/or second geographic region. In this case, the Replikin Count of each isolate of the plurality of isolates from the second time period and/or second geographic region is compared separately to one standard deviation of the mean.

An expansion of influenza isolated in the second time period and/or second geographic region may also be predicted if the number of Replikin Counts of a plurality of isolates from the second period and/or second geographic region that is greater than one standard deviation of the mean is greater than the number of Replikin Counts of said plurality of isolates from the second period and/or second geographic region that is less than one standard deviation of the mean.

The method may also employ a ratio of the number of Replikin Counts that are greater than one standard deviation of the mean divided by the number of Replikin Counts that are less than one standard deviation of the mean. The ratio is called a Replikin Count Virus Expansion Index (RCVE Index). Another way to determine the RCVE Index is to divide the percent of Replikin Counts in a plurality of isolates of influenza virus grouped by time and/or region that are higher than one standard deviation of the mean by the percent of Replikin Counts that are lower than one standard deviation of the mean. An RCVE Index may be used to quantify the future risk of an outbreak of influenza by tracking Replikin Counts in strains of influenza over time.

In determining a RCVE Index, the mean Replikin Count of the plurality of isolates from the first time period and first geographic region is considered a control. A control population preferably has a relatively large number of isolates with a relatively small variability in the Replikin Count of the isolates, but any population may be deemed a control when a comparison between the control and a related isolate or plurality of isolates is desired. A control may be related to the population that is being studied. For example, if influenza infection in a bird species, such as swans, is being studied, the control may be something closely related such as chickens, wherein isolates from chickens may be relatively numerous (if available) and relatively stable (if possible) and wherein stability in Replikin Count through the population demonstrates a level of equilibrium between the expansion and contraction of the strain or related strain of influenza virus in chickens. A control may reflect a highest number of isolates reported in a year or in several years in a geographic area. As may be seen in FIG. 3, Influenza B may be a model control during the 20^{th} century for influenza strains because both Replikin Count and morbidity in all hosts are remarkably stable throughout some 40 years with a relatively small standard deviation and no lethal outbreaks recorded. In influenza B, Replikin Count and replication rate appear to be just sufficient to balance losses for steady survival of the species. This is in contrast to H2N2, which disappeared at the end of the century after dropping Replikin Counts less than one standard deviation of the mean with no Replikin Counts greater than one standard deviation of the mean to balance the survival of the strain.

In determining an RCVE index, any measure of Replikin concentration may be used in influenza or in other pathogens. Replikin Count may reflect the concentration of Replikin peptides identified encoded in the genome of an isolate. Replikin Count may also reflect the concentration of Replikin peptides identified in the expressed proteins of an isolate or in at least one protein or protein fragment of an isolate. Replikin Count may also reflect the concentration of Replikin peptides identified in a Replikin Peak Gene of an isolate. The Replikin Peak Gene of an influenza virus may be any segment of the genome or of any expressed protein or protein fragment having the highest concentration of continuous and/or overlapping Replikin peptides identified.

In many influenza isolates the Replikin Peak Gene is identified in the polymerase area of the influenza virus genome. Within the polymerase area, the Replikin Peak Gene is often identified in the pB1 gene area. Replikin Counts within the pB1 gene may also be used.

Any Replikin peptide, Replikin Peak Gene, protein, protein fragment, or nucleic acid sequence encoding any Replikin peptide, Replikin Peak Gene, protein, or protein fragment in an isolate predicted by the methods of the invention to be expanding may be used for diagnostic, therapeutic, and/or preventive purposes. Further, a vaccine may be manufactured by identifying a portion of the structure or genome of an influenza isolate predicted to expand in population and using that portion in a vaccine composition.

Methods of the invention also provide methods of predicting a decrease in virulence, morbidity, and/or lethality of a strain of influenza and/or predicting a contraction or failure of a strain of influenza wherein a Replikin Count of at least one isolate of a strain of influenza from a second time period and/or second geographic region is less than one standard deviation of the mean of the Replikin Count of a plurality of isolates of influenza from a first time period and first geographic region. A decrease may also be predicted where the number of Replikin Counts of a plurality of isolates from a second period and/or a second geographic region that are greater than one standard deviation of the mean is less than the number of Replikin Counts less than one standard deviation of the mean. A decrease, contraction, or failure is predicted if the ratio of the Replikin Count Virus Expansion Index is less than one.

When a population contains isolates with Replikin Counts above one standard deviation of the mean of a control and does not contain isolates with Replikin Counts below one standard deviation of the mean of the control, the ratio of the RCVE Index is considered to have a denominator of one to avoid an index of infinity.

In determining a Replikin Count Virus Expansion Index, Replikin Counts from Replikin Peak Genes may be analyzed from regions (such as all reporting countries) in a given time period (such as a year) for a range of species. Within a country in a year, there may be a range of values over a range of species. The ordinary skilled artisan may select a mean Replikin Count as a control from the range of values, a time, a region, a species, or any combination thereof (such as a time, a region, and a species, e.g., 2004, China, and chicken). For example, in Example 7 below, the mean Replikin Count of all H5N1 isolates from chickens in China in 2004 was selected as an initial control against which Replikin Counts from swans in China in 2004 were compared. When comparing a control to the Replikin Count of an individual isolate or related group of isolates, a control that shares some similarity with the isolate or group of isolates may be used. For example, a control of all isolates from chicken in China in 2004 may be compared with other isolates from 2004. Likewise, a control of swans from 2005 in Japan may be compared to future isolates from swans in Japan. The ordinary skilled artisan will understand when a control shares similarity with an isolate or group of related isolates such that the control may be used in comparison with the isolate or group of related isolates.

When comparing the Replikin Count of an individual isolate or related group of isolates to a control, all Replikin Count values within the group of related isolates that fall within one standard deviation of the mean may be treated as a group. Additionally, all values that fall outside the range of one standard deviation from the mean may be treated as two outlying groups. A first group is the group of Replikin Counts that are greater than the mean plus one standard deviation. A second group is the group of Replikin Counts that are less than the mean minus one standard deviation. Because higher Replikin Counts are associated with future outbreaks or an expanding virus population and lower Replikin Counts are associated with cessation of outbreaks or decrease or failure of the virus population, the ratio of the percent of isolates having Replikin Counts above mean plus one standard deviation to the percent of isolates having Replikin Counts below the mean minus one standard deviation provides a quantitative index of the viability and expansion of the virus. The index provides a snapshot of current status of the virus population and the propensity for change in that population. If the ratio is greater than one, the RCVE Index predicts an expanding population. If the ratio is less than one, the RCVE Index predicts a contracting or failing virus population.

### Mitigating and Treating Expanding Populations of Influenza Virus

One aspect of the present invention provides methods of preventing or treating outbreaks of influenza virus by predicting an expansion of a strain of influenza virus using a Replikin Count Virus Expansion Index and administering therapies comprising an isolated or synthesized portion of the structure or genome of the influenza virus identified using the RCVE Index to prevent, mitigate, or treat the outbreak of influenza virus. A prediction of an outbreak may be made by (1) determining a mean Replikin Count with standard deviation for a group of isolates of a strain of influenza isolated during a first time period in a first geographic region, (2) determining a Replikin Count of at least one isolate of the same strain of influenza virus from a second time period and/or second geographic region that is different from the first time period and/or is different from the second geographic region, and (3) predicting an expansion of the strain of influenza isolated in said second time period and/or second geographic region if the Replikin Count of the isolate from a second time period and/or second geographic region is greater than one standard deviation from the mean of the Replikin Count of the plurality of isolates isolated in the first time period and in the first geographic region. An outbreak may be prevented, mitigated, or treated by administering a pharmaceutical compound that includes all or some portion of the structure or genome of the at least one isolate of influenza virus.

The at least one isolate of influenza from a second time period and/or geographic region may be a plurality of isolates from the second time period and/or second geographic region wherein the Replikin Count of each isolate of the plurality of isolates is compared separately to one standard deviation from the mean. Additionally, an outbreak of influenza may be predicted if the number of Replikin Counts of the plurality of isolates from a second period and/or a second geographic region that is greater than one standard deviation of the mean is greater than the number of Replikin Counts less than one standard deviation of the mean.

The portion of the structure or genome may be isolated from an influenza isolate or may be synthesized based on sequences or other structure elucidated from the influenza isolate as well understood by the ordinary skilled artisan. The structure may be a protein or protein fragment that comprises a Replikin peptide or that consists of a Replikin peptide. The structure may comprise or consist of a Replikin Peak Gene or a fragment of a Replikin Peak Gene or may consist of a Replikin peptide identified within a Replikin Peak Gene. The structure may also be a nucleic acid including but not limited to a nucleic acid encoding a Replikin Peak Gene, a Replikin peptide or plurality of Replikin peptides within a Replikin Peak Gene, or a Replikin peptide or plurality or Replikin peptides.

A peptide or mixture of peptides may be comprised in an immunogenic compound for influenza and may include at least one of HAQDILEKEHNGKLCSLKGVRPLILK (SEQ ID NO: 1), KEHNGKLCSLKGVRPLILK (SEQ ID NO: 2), KKNNAYPTIKRTYNNTNVEDLLIIWGIHH (SEQ ID NO: 3), HHSNEQGSGYAADKESTQKAIDGITNK (SEQ ID NO: 4), HDSNVKNLYDKVRLQLRDNAK (SEQ ID NO: 5), KVRLQLRDNAKELGNGCFEFYH (SEQ ID NO: 6), KDVMESMDKEEMEITTH (SEQ ID NO: 7), HFQRKRRVRDNMTKK (SEQ ID NO: 8), KKWSHKRTIGKKKQRLNK (SEQ ID NO: 9), HKRTIGKKKQRLNK (SEQ ID NO: 10), HEGIQAGVDRFYRTCKLVGINMSKKK (SEQ ID NO: 11); or HSWIPKRNRSILNTSQRGILEDEQMYQKCCNLFEK (SEQ ID NO: 12).

### Synchronous Replikin Cycles in H9N2 and H5N1 Strains of Influenza

Another aspect of the invention provides methods of predicting an increase in the virulence, morbidity, and/or lethality or an expansion of the population of an isolate of a strain of influenza virus as compared to another isolate or group of isolates of the same or a related strain. Such an increase may be predicted by identifying a cycle of Replikin concentration among a plurality of isolates of influenza and identifying a peak in that cycle. An increase is predicted following the time point or time period when the peak is identified or following a rising portion of the cycle. An increase may likewise be predicted following the time point or time period when a peak is identified in two synchronous cycles wherein a first cycle is the cycle of a strain of influenza and the second cycle is a cycle of a different strain of influenza. The increase is predicted following the time period in which the peaks of the synchronous cycles are identified or in a rising portion identified in both synchronous cycles.

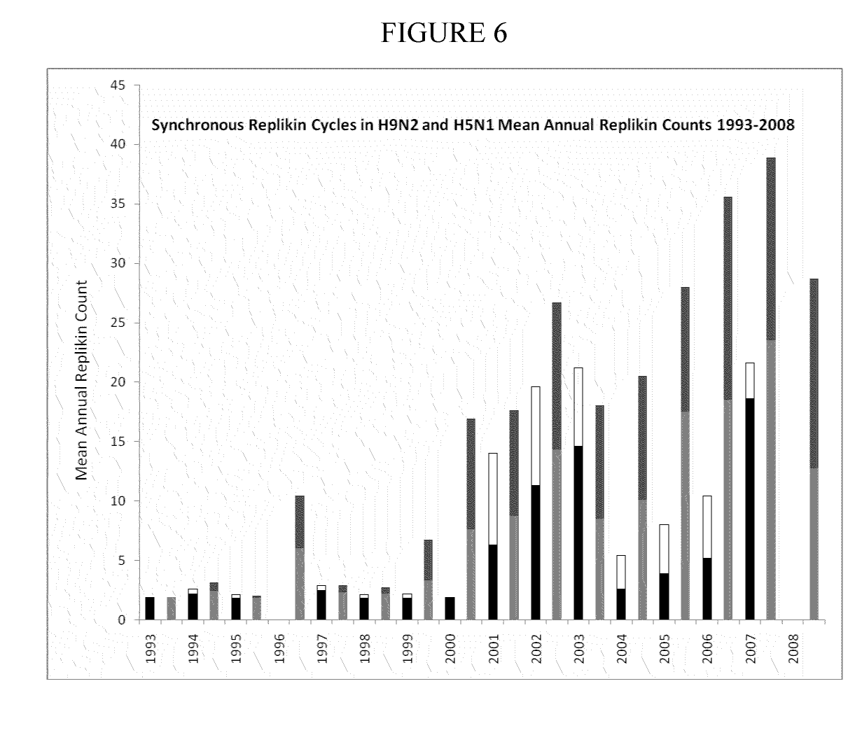

A cycle of Replikin concentration or “Replikin cycle” of H9N2 may be seen in FIG. 5. A comparison of synchronized cycles of Replikin concentration in H5N1 and H9N2 may be seen in FIG. 6. The synchronized cycles in these two influenza strains correspond to and retrospectively predict H5N1 outbreaks in 1997, 2001, 2004, 2007 and the present outbreak in 2008 and 2009.

In FIG. 5, the mean annual Replikin Count of the pB1 gene area of H9N2 is shown in light gray columns with standard deviation shown in dark gray columns above the H9N2 annual mean Replikin Count. The standard deviation data emphasize the extent of the expanding Replikin Counts within the annual population. The number of poultry flocks reported in Israel with H9N2 infection is provided in white columns. In FIG. 6, mean annual Replikin Count for H9N2 is again reported in light gray columns with standard deviation reported above in dark gray columns. Mean annual Replikin Count for H5N1 is reported in black columns with standard deviation reported in white columns above the H5N1 annual mean Replikin Count. FIG. 6 visibly illustrates synchrony between the H9N2 and H5N1 Replikin Cycles.

The data for FIGS. 5 and 6 are disclosed in Table 4 below. In Table 4, mean annual Replikin Count with standard deviation are provided for all amino acid sequences publicly available at www.pubmed.com for H9N2 and H5N1 strains of influenza isolated from 1993 through 2008. The number of poultry flocks reported to have H9N2 infections in Israel are also disclosed for years 2000 through 2004 as a measure of outbreaks of H9N2.

TABLE 4
Synchronous Replikin Cycles in H9N2 and H5N1

| | H9N2 pB1 | | Infected | H5N1 pB1 | |
|---|---|---|---|---|---|
| | gene area | | Poultry | gene area | |
| | Mean | Standard | Flocks | Mean | Standard |
| | Replikin | Deviation | Israel | Replikin | Deviation |
| Year | Count | H9N2 | (×½) | Count | H5N1 |
| 1993 | 1.9 | 0.0 | | 1.9 | 0.0 |
| 1994 | 2.4 | 0.7 | | 2.2 | 0.4 |
| 1995 | 1.8 | 0.2 | | 1.8 | 0.3 |
| 1996 | 6.0 | 4.4 | | | |
| 1997 | 2.3 | 0.6 | | 2.5 | 0.4 |
| 1998 | 2.2 | 0.5 | | 1.8 | 0.3 |
| 1999 | 3.3 | 3.4 | | 1.8 | 0.4 |
| 2000 | 7.6 | 9.3 | 5.0 | 1.8 | 0.1 |
| 2001 | 8.7 | 8.9 | 4.5 | 6.3 | 7.7 |
| 2002 | 14.3 | 12.4 | 12.5 | 11.3 | 8.3 |
| 2003 | 8.5 | 9.5 | 48.5 | 14.6 | 6.6 |
| 2004 | 10.1 | 10.4 | 42.5 | 2.6 | 2.8 |
| 2005 | 17.5 | 10.5 | | 3.9 | 4.1 |
| 2006 | 18.5 | 17.1 | | 5.2 | 5.2 |
| 2007 | 23.5 | 15.4 | | 18.6 | 3.0 |
| 2008 | 12.7 | 16.0 | | | |

As illustrated in FIGS. 5 and 6 with data provided above in Table 4, the H9N2 strain of influenza, which commonly infects poultry and occasionally infects humans, has been found to have completed a second five year Replikins expansion cycle in which H9N2 Replikin Counts of Replikin peptides identified as encoded in the pB1 region of the influenza genome reached levels twice those found in H5N1. As may be seen in the figures, H9N2 Replikin Counts increased in 1996, one year before the H5N1 outbreak in Hong Kong in 1997. In 1999, increasing Replikin Counts in the pB1 region of H9N2 also preceded increases in Replikin Counts in the pB1 region of H5N1 as well as H5N1 outbreaks. As may be seen in FIG. 6, the Replikin Cycles of H9N2 and H5N1 coincide and share a visible level of synchrony. Further, as may be seen from FIG. 6, the Replikin Count level for H9N2 has, as of 2008, increased in concentration more than the Replikin Count level for H5N1. As such, while not wishing to be bound by theory, it is noteworthy that H9N2 strains of influenza and H5N1 strains of influenza appear to have a synchronized cyclic precursor-competitor evolutionary biochemical relationship. The data predict that H9N2 is an alternate candidate to H5N1 for a future influenza pandemic.

In FIGS. 5 and 6, each cycle of H9N2 or H5N1 is defined by Replikin Counts (number of Replikin peptides per 100 amino acids) of specific Replikin peptides in the pB1 gene area. An increase in successive years followed by a decrease in successive years is observable. FIG. 5 illustrates that increasing H9N2 Replikin Counts precede the occurrence of increasing numbers of H9N2 infections in poultry flocks. FIG. 5 further demonstrates that Replikin Counts in H9N2 began to increase again in 1999, two years before a reported increase of H9N2 outbreaks in poultry in the Middle East, including Israel. As may be seen in FIG. 6, following the increase in H9N2, Replikin Counts began to increase in H5N1 in 2000 with infections beginning in 2000 and forward.

The H9N2 sequences analyzed and reported as mean Replikin Count in Table 4 and in FIGS. 5 and 6 include all those published on PubMed worldwide. A principal portion of the sequences are from influenza isolated in China and the Middle East.

Two Replikin Count expansion rising portions of cycles are seen in FIG. 6 with visible synchrony. The first expansion rising portion of a cycle is observed from 1999 to 2003. The second expansion rising portion of a cycle is observed from 2004 to 2008. In the second rising portion, the maximum Replikin Counts for H9N2 were greater than those in its first rising portion and double the maximum Replikin Counts seen in H5N1. The maximum Replikin Counts observed for H9N2 are likewise double the maximum Replikin Counts observed for any other influenza strain so far analyzed. See, e.g., FIGS. 7 and 8.

Additionally, the standard deviations for H9N2 as illustrated in FIGS. 5 and 6 are clearly greater than the standard deviations for the H5N1 values, indicating greater activity in Replikin Count in H9N2. This observable up-regulation of H9N2 Replikin Peak Gene area through observable changes in Replikin Count is seen in advance of H9N2 outbreaks. A similar trend is observable in Replikin Counts in West Nile Virus for viruses isolated from 2000 through 2008. See FIG. 3. Likewise, predictive cycles have been noted in malaria, foot and mouth disease and other influenza strains. See, id. FIGS. 1-6.

The data in Table 4 and FIGS. 5 and 6 predict that additional increases in both Replikin Counts and consequent H5N1 and H9N2 infections may be expected in a coming third Replikin Count cycle in H9N2 and H5N1. An outbreak of H5N1 in chickens in Hong Kong in early December 2008 and a reported H9N2 infection in a child in Hong Kong in late December 2008 substantiates the predictive capacity of the data. Other H5N1 outbreak data from the Assam, Meghalaya, and West Bengal regions of Indian in late 2008 further substantiate the prediction.

### Vaccines, Treatments and Therapeutics

The observations of specific Replikins and their concentration in malaria, West Nile virus, foot and mouth disease virus, and influenza virus proteins provides specific quantitative early chemical correlates of outbreaks and increases in mortality and provides for production and timely administration of vaccines tailored specifically to treat the prevalent emerging or re-emerging strain virus in a particular region of the world. Synthesis of these vaccines may be accomplished in seven days or less, which allows for administration of vaccines that are a best fit for a particular virulent strain of virus or organisms including malarial trypanosomes, West Nile virus, foot and mouth disease virus, and influenza virus.

By analyzing the protein sequences of isolates of a virus or other pathogen for the presence, concentration and/or conservation of Replikins, pandemics, epidemics, and other changes in virulence and mortality can be predicted and treatments developed. Furthermore, the severity of such outbreaks can be significantly lessened by administering a peptide vaccine based on the Replikin sequences found to be most abundant or shown to be on the rise in virus isolates over a given time period, such as about one to about three years.

A peptide vaccine of the invention may include a single Replikin peptide sequence or may include a plurality of Replikin sequences observed in particular virus strains. However, a vaccine may include a conserved Replikin peptide(s) in combination with a new Replikin(s) peptide or may be based on new Replikin peptide sequences. The Replikin peptides can be synthesized by any method, including chemical synthesis or recombinant gene technology, and may include non-Replikin sequences, although vaccines based on peptides containing only Replikin sequences are preferred. Preferably, vaccine compositions of the invention also contain a pharmaceutically acceptable carrier and/or adjuvant. Among the Replikin peptides for use in a virus or pathogen vaccine are those Replikins observed to “re-emerge” after an absence from the amino acid sequence for one or more years.

The vaccines of the present invention can be administered alone or in combination with antiviral drugs, such as gancyclovir; interferon; interleukin; M2 inhibitors, such as, amantadine, rimantadine; neuraminidase inhibitors, such as zanamivir and oseltamivir; and the like, as well as with combinations of antiviral drugs.

The vaccine of the present invention may be administered to any animal capable of producing antibodies in an immune response. For example, the vaccine of the present invention may be administered to a rabbit, a chicken, a shrimp, a pig, or a human. Because of the universal nature of Replikin sequences, a vaccine of the invention may be directed at a range of strains of a virus or organism or a particular strain of virus or organism.

The Replikin peptides of the invention, alone or in various combinations are administered to a subject, in a non-limited embodiment by i.v., intramuscular injection, by mouth, or by spray inhalation, intranasal administration, or intraocular administration. The peptides are administered in order to stimulate the immune system of the subject to produce antibodies to the peptide. Generally the dosage of peptides is in the range of from about 0.01 μg to about 500 mg, about 0.05 μg to about 200 mg, or about 0.075 μg to about 30 mg, from about 0.09 μg to about 20 mg, from about 0.1 μg to about 10 mg, from 10 μg to about 1 mg, and from about 50 μg to about 500 μg. The skilled practitioner can readily determine the dosage and number of dosages needed to produce an effective immune response.

In another aspect of the invention, isolated Replikin peptides may be used to generate antibodies, which may be used, for example to provide passive immunity in an individual or for diagnostics. See, e.g., U.S. application Ser. No. 11/355,120, filed Feb. 16, 2006 and U.S. application Ser. No. 12/010,027, filed Jan. 18, 2008 (each incorporated herein by reference in their entirety).

### Example 1

### Analysis of Replikin Count in Malaria to Predict Increased Mortality

Publicly available sequences of isolates of P. falciparum at www.pubmed.com were analyzed using proprietary search tool software (ReplikinForecast™ available in the United States from REPLIKINS LLC, Boston, Mass.) for years 1986 to 2007 to determine the mean Replikin Count for the histidine-rich protein of all isolates available in each of those years. Mean annual Replikin Counts for each year were then compared with changes in mortality as reported by the World Health Organization.

A list of the accession numbers analyzed for the presence and concentration of Replikin sequences is provided in Table 5 below. The mean Replikin Count for each year is provided following the list of accession numbers from isolates in each corresponding year. Standard deviation and significance as compared to the mean Replikin Count of the previous year and of the lowest mean Replikin Count within the data set are also provided along with the mean Replikin Count for each year.

TABLE 5
Malaria Annual Mean Replikin Count

| | | | Mean | | |
|---|---|---|---|---|---|
| | | No. of | Replikin | | |
| | | Isolates | Count | | |
| Year | PubMed Accession Number-Replikin Count | per year | per year | S.D. | Significance |
| 1986 | AAA51639 25 P09346 295 P05227 25 P05228 23 AAA29617 | 6 | 15.9 | 15.2 | low p > 0.50 |
| | 23 AAA29631 37 | | | | |
| 1987 | P06719 307 P13817 268 AAA29630 268 P05229 236 | 11 | 38.2 | 23.5 | low p < 0.005, |
| | CAA68268 307 AAA29629 295 AAA29621 15 AAA29620 33 | | | | prev p < 0.02 |
| | P13825 33 P14588 15 AAA73197 135 | | | | |
| 1988 | | | | | |
| 1989 | CAA01078 23 | 1 | 13.9 | 0.0 | prev p < 0.005 |
| 1990 | AAA74651 19 | 1 | 5.2 | 0.0 | 1990 |
| 1991 | | | | | |
| 1992 | CAA49542 21 CAA49548 23 CAA49547 23 CAA49546 23 | 9 | 13.0 | 18.2 | low p > 0.50, |
| | CAA49545 23 CAA49543 23 CAA49544 23 AAA29739 204 | | | | prev p < 0.20 |
| | NP_001772 11 | | | | |
| 1993 | | | | | |
| 1994 | | | | | |
| 1995 | CAK38915 9 | 1 | 2.4 | 0.0 | prev p < 0.10 |
| 1996 | AAC47454 23 AAC47453 23 CAB01211 32 | 3 | 8.7 | 1.2 | low p < 0.01, |
| | | | | | prev p < 0.01 |
| 1997 | | | | | |
| 1998 | XP_001349534 295 AAC71810 295 XP_001349702 279 | 7 | 24.1 | 16.7 | low p < 0.10, |
| | AAC71973 279 AAD40570 45 AAD40569 31 AAD20952 52 | | | | prev p < 0.04 |
| 1999 | CAD49281 1366 AAD23574 249 AAD31511 23 AAF14632 23 | 4 | 62.9 | 62.9 | low p < 0.20, |
| | | | | | prev p < 0.20 |
| 2000 | AAF74261 290 AAG01323 295 AAF74262 22 | 3 | 33.3 | 24.7 | low p < 0.30, |
| | | | | | prev p < 0.40 |
| 2001 | | | | | |
| 2002 | XP_001351550 1366 CAG25049 193 XP_001348072 121 | 13 | 18.0 | 29.0 | low p > 0.50, |
| | XP_001347535 485 XP_001350735 874 AAN35985 121 | | | | prev p < 0.30 |
| | AAN35448 485 AAN36415 874 XP_960846 25 EAA31610 25 | | | | |
| | CAD36995 25 XP_726238 110 EAA17803 110 | | | | |
| 2003 | AAQ63567 87 AAQ63566 64 AAQ63565 63 AAQ63564 66 | 7 | 28.4 | 3.0 | low p < 0.001, |
| | AAQ63563 66 AAQ63562 66 AAQ63561 67 | | | | prev p < 0.20 |
| 2004 | XP_966219 193 | 1 | 17.0 | 0.0 | prev p < 0.001 |
| 2005 | EDL47342 352 EDL45776 150 XP_763898 7 AAW78557 234 | 5 | 7.4 | 6.5 | low p < 0.05, |
| | EAN31615 7 | | | | prev p < 0.02 |
| 2006 | | | | | |
| 2007 | ABU43157 23 BAF93906 2 ZP_02691628 36 XP_001617069 | 8 | 17.2 | 19.0 | low p > 0.50, |
| | 352 XP_001615503 150 XP_001615499 369 XP_001639181 | | | | prev p < 0.20 |
| | 505 EDO47118 505 | | | | |

Analysis of the annual mean Replikin Count of the histidine rich protein between 1986 and 2007 revealed cycles of Replikin Count. The beginning of a new cycle provides a prediction that Replikin Count may continue to increase along with an increase in malaria mortality rate. The data is graphically illustrated in FIG. 1 and summarized in Table 1 above.

Replikin peptides in an isolate identified at a peak or in a rising portion of the Replikin cycles revealed in FIG. 1 are available as peptides of the invention. For example, any Replikin peptide identified in the ABU43157 accession number of an isolate from 2007 is available as a diagnostic, therapeutic, or preventive compound or composition of the invention because it is identified in a rising portion of a Replikin cycle. Replikin peptides identified in the 1999 isolate reported at accession number CAD49281 are likewise available. See FIG. 1 and Table 2. The 1999 isolate is present at the peak of a Replikin cycle, as such, Replikin peptides identified in the isolate reported at CAD49281 may be used as immunogenic compounds. Additionally, the 1998 accession number XP001349534 is identified from an isolate in a rising portion in a Replikin cycle. See FIG. 1 and Table 5. Replikin peptides identified in ABU43157, CAD49281, and XP001349534, among others, are likewise useful as immunogenic compounds or vaccines or for diagnosis or treatment of malaria.

### Example 2

### Analysis of Replikin Count in Malaria ATP-ase to Predict Increased Mortality

Applicants analyzed publicly available sequences of the ATP-ase enzyme of isolates of P. falciparum at www.pubmed.com. The data is summarized below in Table 6 and illustrated in FIG. 2. The data illustrate that mortality rates per 1000 clinical cases of malaria in humans correlate with annual mean Replikin Count in sequences of the P. falciparum ATP-ase enzyme publicly available at www.pubmed.com. Replikin Counts of P. falciparum ATP-ase increased from 1997 to 1998 along with an increase in mortality per malaria case from 1997 and 1998 to 1999. The Replikin Count of P. falciparum ATP-ase decreased from 1998 to 2006 along with mortality rates from 1999 to 2005 (consistent mortality presently available only through 2005). High malaria morbidity and mortality rates occurred in the late 1990s and were thought to be due to adaptation of the microorganism and decreased effectiveness of anti-malarials. ATP-ase is a primary target of arteminisin treatment of malaria. With increased use of arteminisin, and improved public health measures, morbidity and mortality rates declined from 1999 to 2005. Mortality rates in Table 6 are recorded as declared by the World Health Organization. See www.who.int.

TABLE 6

| | Mean | | |
|---|---|---|---|
| | Replikin | | Mortality Rate |
| | Count in P. falciparum | Standard | per 1000 |
| Year | ATP-ase | Deviation | Malaria Cases |
| 1997 | 19 | 7.7 | 17 |
| 1998 | 19.4 | 16.6 | 17 |
| 1999 | 16.1 | 9.1 | 19 |
| 2000 | 11.2 | 10.5 | 16 |
| 2001 | 7.7 | 8.1 | 13 |
| 2002 | 12.7 | 9.9 | 10 |
| 2003 | 3.3 | 2.5 | 10 |
| 2004 | 4.2 | 4.6 | 9 |
| 2005 | 6.3 | 3.9 | 9 |
| 2006 | 3.4 | 2.6 | |
| 2007 | 6.2 | 8.4 | |

### Example 3

### Analysis of Replikin Count Cycles in West Nile Virus Predict Increased Morbidity

Envelope protein sequences from isolates of West Nile virus isolated between 2000 and 2008 that were publicly available at www.pubmed.com were analyzed for Replikin sequences and a mean annual Replikin Count was determined. The data are contained in Table 7 below and illustrated in FIG. 3.

FIG. 3 illustrates cycling of mean annual Replikin Count in West Nile virus in correlation with cycling of West Nile virus morbidity. Cycles are detectable because of repeating conserved virus structures and continuity of the Replikin phenomenon through time. The mean annual Replikin Count of the Envelope Protein of WNV (black), and standard deviation, is compared to the annual number of human cases in the U.S. per CDC reports (gray).

2000 to 2003: The standard deviation of the mean of the Replikin Count of the envelope protein increases markedly from 2000 to 2001 (p<0.001). This change has been observed in all common strains of influenza virus (not the same virus genus as WNV) to signal rapid replication and expansion of the range of the Replikin Count, thus virus population expands with Replikin Count and precedes virus outbreak. The increase in the mean Replikin Count from 2000 to 2003 appears to accompany, or precede, the increase in the number of human WNV cases recorded independently and published by the Center for Disease Control (CDC). The same relationship of Replikin Count to morbidity has been shown in influenza strains, for example H5N1 to human mortality, and in H3N8 equine encephalitis to horse morbidity, and in the trypanosome Plasmodium falciparum (malaria) to human morbidity, and to mortality rate in shrimp with shrimp taura syndrome virus. Since the relationship has already been demonstrated in several species, including crustaceans, horses, and humans, it appears to be a broadly distributed general principle. 2004 to 2007: In 2004 and 2005, there was a decrease from 2003 in both the Replikin Count and the number of human cases of WNV. In 2006, there was an increase in the Replikin Count followed by an increase in 2007 of the number of human cases.

In FIG. 3, cycles of Replikin concentration and cycles of WNV human morbidity may be observed to correlate. Until the present data, it was not understood that cycles within a particular strain of pathogen actually continued from a peak to a trough to another peak to another trough. Instead, in the past, it was understood that an increase in Replikin concentration correlated with outbreaks and a decrease in Replikin concentration correlated with retraction. With these new data, however, it is now understood and contemplated by the invention that entire Replikin cycles from peak to trough to peak to trough and/or from trough to peak to trough to peak correlate with cycles in virulence, morbidity, and mortality. The invention now provides methods of tracking pathogens as they increase in virulence, expand in population within a region or into a region, or increase in morbidity or mortality by monitoring changes in Replikin concentration.

The rising numbers for both the Replikin Count and the number of cases in the second rising portion of the cycle, 2004-2008, when compared to the first rising portion of the cycle, suggests an increased or ‘improved’ infective efficiency accompanying an increased Replikin Count in the second rising portion, compared to the first. The drop in efficacy of the virus is probably due to the generation of resistance in the host; the subsequent rise in infectivity in the second rising portion of the cycle is related to the appearance of new Replikins identified in WNV. Once again, the close relationship of Replikins to infectivity is demonstrated; both literally rise and fall together.

Thus the present data provide direct quantitative evidence of the relationship of Replikins to infectivity at a more accurate level than previously available. For example, in the case of H5N1 influenza, the cycle began in 1996, with the Hong Kong outbreak. It was temporarily ended in 1998 by the complete culling of chickens in Hong Kong. The H5N1 clinical ‘sub-cycle’ resumed in 2000, continued to the present, and was predicted prospectively each year by the Replikin Count. In this case, occurring mostly in East Asian countries, H5N1 was not as subject to exact epidemiological reports by the WHO of morbidity and mortality as in the case of West Nile Virus in the U.S. as here presented, since the CDC keeps much more accurate surveillance records of the morbidity and mortality.

While not wishing to be limited by theory, the close relationship of Replikin Count to morbidity and mortality, and other evidence, has led to the hypothesis that Replikins, in addition to being closely involved in the biochemistry of rapid replication, are in fact infective units, that the viruses and trypanosomes are merely carriers of the Replikin infective units, but that other virus or trypanosome structures are needed to produce infectivity in the host.

FIG. 3 illustrates that early detection of changes in Replikin Count may be directly translated in a rapid response with vaccines to the emerging Replikin structures that may be synthesized in seven days or fewer after identification of the emerging Replikin sequences using, for example, ReplikinForecast™ software (Replikins LLC Boston, Mass.).

Accession numbers, number of isolates, mean Replikin Count, standard deviation and significance for accession numbers available for West Nile virus envelope protein from www.pubmed.com are contained below in Table 7. Specific conserved Replikin sequences identified within the step-wise cycles of West Nile virus in FIG. 3 include, KIIQKAHK (SEQ ID NO: 13), HLKCRVKMEK (SEQ ID NO: 14), KLTSGHLK (SEQ ID NO: 15), and HNDKRADPAFVCK (SEQ ID NO: 16). The accession numbers in which these sequences are conserved are listed in Example 6 of U.S. application Ser. No. 12/108,458, filed Apr. 23, 2008, which is incorporated herein by reference in its entirety.

TABLE 7
West Nile Virus Envelope Protein Replikin Count Cycles

| | | | Mean | | |
|---|---|---|---|---|---|
| | | | Replikin | | |
| | PubMed Accession Number | No. of Isolates per | Count per | | |
| Year | Replikin Count West Nile Virus Envelope Protein | year | year | S.D. | Significance |
| 2000 | ABR19638 102 AAK06624 97 AAG02039 98 AAG02038 97 | 4 | 2.9 | 0.1 | low p < 0.001, |
| | | | | | prev p < 0.001 |
| 2001 | AAM70028 28 AAL07765 6 AAL07764 6 AAL07763 6 AAL07762 6 | 130 | 3.6 | 2.0 | low p < 0.02, prev |
| | AAL07761 6 AAL14222 30 AAL14221 30 AAL14220 30 AAL14219 | | | | p < 0.001 |
| | 30 AAL14218 30 AAL14217 30 AAL14216 30 AAL14215 30 | | | | |
| | AAK58104 30 AAK58103 31 AAK58102 30 AAK58101 30 | | | | |
| | AAK58100 30 AAK58099 31 AAK58098 30 AAK58097 30 | | | | |
| | AAK58096 30 AAK52303 30 AAK52302 30 AAK52301 30 | | | | |
| | AAK52300 30 AAK62766 32 AAK62765 32 AAK62764 32 | | | | |
| | AAK62763 32 AAK62762 32 AAK62761 32 AAK62760 32 | | | | |
| | AAK62759 32 AAK62758 32 AAK62757 32 AAK62756 32 | | | | |
| | AAK91592 20 ABR19637 111 AAM81753 97 AAM81752 97 | | | | |
| | AAM81751 97 AAM81750 97 AAM81749 97 AAK67141 7 | | | | |
| | AAK67140 7 AAK67139 7 AAK67138 7 AAK67137 7 AAK67136 7 | | | | |
| | AAK67135 7 AAK67134 7 AAK67133 7 AAK67132 7 AAK67131 7 | | | | |
| | AAK67130 7 AAK67129 7 AAK67128 7 AAK67127 7 AAK67126 7 | | | | |
| | AAK67125 7 AAK67124 3 AAK67123 7 AAK67122 7 AAK67121 7 | | | | |
| | AAK67120 7 AAK67119 7 AAK67118 7 AAK67117 7 AAK67116 7 | | | | |
| | AAK67115 7 AAK67114 7 AAK67113 7 AAK67112 7 AAK67111 7 | | | | |
| | AAK67110 7 AAK67109 7 AAK67108 7 AAK67107 7 AAK67106 7 | | | | |
| | AAK67105 7 AAK67104 7 AAK67103 7 AAK67102 7 AAK67101 7 | | | | |
| | AAK67100 7 AAK67099 7 AAK67098 7 AAK67097 7 AAK67096 7 | | | | |
| | AAK67095 7 AAK67094 7 AAK67093 7 AAK67092 7 AAK67091 7 | | | | |
| | AAK67090 7 AAK67089 7 AAK67088 7 AAK67087 7 AAK67086 7 | | | | |
| | AAK67085 7 AAK67084 7 AAK67083 7 AAK67082 7 AAK67081 7 | | | | |
| | AAK67080 7 AAK67079 7 AAK67078 7 AAK67077 7 AAK67076 7 | | | | |
| | AAK67075 7 AAK67074 7 AAK67073 7 AAK67072 7 AAK67071 7 | | | | |
| | AAK67070 5 AAK67069 7 AAK67068 7 AAK67067 7 AAK67066 7 | | | | |
| | AAK67065 7 AAK67064 7 AAL87748 19 AAL87747 18 AAL87746 | | | | |
| | 19 AAL87745 18 AAL37596 18 AAM21944 24 AAM21941 32 | | | | |
| 2002 | AAM09856 6 AAM09855 6 AAM09854 6 AAO26579 30 AAO26578 | 18 | 4.7 | 1.5 | low p < 0.001, |
| | 30 AAN77484 3 AAN85090 97 AAO73303 36 AAO73302 36 | | | | prev p < 0.005 |
| | AAO73301 36 AAO73300 36 AAO73299 36 AAO73298 36 | | | | |
| | AAO73297 36 AAO73296 36 AAO73295 36 AAL87234 96 | | | | |
| | CAD60131 96 | | | | |
| 2003 | AAP20887 96 AAR10793 6 AAR10784 6 AAR17575 32 AAR17574 | 94 | 5.3 | 1.5 | low p < 0.001, |
| | 32 AAR17573 32 AAR17572 32 AAR17571 32 AAR17570 32 | | | | prev p < 0.05 |
| | AAR17569 32 AAR17568 32 AAR17567 32 AAR17566 32 | | | | |
| | AAR17565 32 AAR17564 32 AAR17563 32 AAR17562 32 | | | | |
| | AAR17561 32 AAR17560 32 AAR17559 32 AAR17558 32 | | | | |
| | AAR17557 32 AAR17556 32 AAR17555 32 AAR17554 32 | | | | |
| | AAR17553 32 AAR17552 32 AAR17551 32 AAR17550 32 | | | | |
| | AAR17549 32 AAR17548 32 AAR17547 32 AAR17546 32 | | | | |
| | AAR17545 32 AAR17544 32 AAR17543 32 AAR17542 32 | | | | |
| | AAQ87608 16 AAQ87607 16 AAQ87606 14 AAR10804 6 AAR10803 | | | | |
| | 6 AAR10802 6 AAR10801 6 AAR10800 6 AAR10799 6 AAR10798 6 | | | | |
| | AAR10797 6 AAR10796 6 AAR10795 6 AAR10794 6 AAR10792 6 | | | | |
| | AAR10791 6 AAR10790 6 AAR10789 6 AAR10788 6 AAR10787 6 | | | | |
| | AAR10786 6 AAR10785 6 AAR10783 6 AAR10782 6 AAR10781 6 | | | | |
| | AAR10780 6 AAQ88403 10 AAQ88402 10 AAX99361 97 AAR84198 | | | | |
| | 36 AAQ55854 97 AAR14153 36 AAR84614 95 AAR06948 36 | | | | |
| | AAR06947 36 AAR06946 36 AAR06945 36 AAR06944 36 | | | | |
| | AAR06943 36 AAR06942 36 AAR06941 36 AAR06940 36 | | | | |
| | AAR06939 36 AAR06938 36 AAR06937 36 AAR06936 35 | | | | |
| | AAR06935 36 AAR06934 36 AAR06933 36 AAR06932 36 | | | | |
| | AAR06931 36 AAQ00999 100 AAQ00998 97 AAP22087 97 | | | | |
| | AAP22086 97 AAP22089 97 AAP22088 96 | | | | |
| 2004 | AAT11553 32 AAT11552 32 AAT11551 32 AAT11550 32 AAT11549 | 55 | 4.2 | 1.7 | low p < 0.001, |
| | 32 AAT11548 32 AAT11547 32 AAT11546 32 AAT11545 32 | | | | prev p < 0.001 |
| | AAT11544 32 AAT11543 32 AAT11542 32 AAT11541 32 AAT11540 | | | | |
| | 32 AAT11539 32 AAT11538 32 AAT11537 32 AAT11536 32 | | | | |
| | AAT11535 32 AAT11534 28 AAS75296 6 AAS75295 6 AAS75294 6 | | | | |
| | AAS75293 6 AAS75292 6 AAS75291 6 AAT95390 108 AAU00153 | | | | |
| | 96 AAV54504 97 AAT02759 111 ABG67747 99 ABG67746 99 | | | | |
| | BAD34491 97 BAD34490 97 BAD34489 97 BAD34488 97 | | | | |
| | ABV82765 97 AAZ91684 106 AAW56064 97 AAW56066 97 | | | | |
| | AAW56065 97 AAW28871 97 AAV49728 6 AAV49727 6 AAV49726 | | | | |
| | 6 AAV49725 6 AAV49724 6 AAT92099 97 AAT92098 97 AAV52690 | | | | |
| | 96 AAV52689 97 AAV52688 97 AAV52687 97 AAV68177 97 | | | | |
| | AAX09982 97 | | | | |
| 2005 | YP_001527880 32 ABC18309 8 ABC18308 9 ABC02196 3 | 125 | 4.3 | 1.8 | low p < 0.001, |
| | AAY67877 9 AAY67876 11 AAY67875 11 AAY67874 8 AAY67873 | | | | prev p > 0.50 |
| | 8 AAY67872 8 AAY67871 8 AAY67870 8 AAY67869 8 AAY67868 8 | | | | |
| | AAY67867 8 AAY67866 8 AAY57985 8 ABB01532 97 ABC40712 | | | | |
| | 100 YP_001527877 97 ABB01533 101 ABA62343 97 AAY32590 36 | | | | |
| | AAY32589 36 YP_001527879 4 AAY55949 97 AAY29684 6 | | | | |
| | AAY29685 6 AAY29683 6 AAY29682 6 AAY29681 6 AAY29680 6 | | | | |
| | AAY29679 6 AAY29678 6 AAY29677 7 AAY29676 7 AAZ32750 97 | | | | |
| | AAZ32749 97 AAZ32748 94 AAZ32747 94 AAZ32746 94 AAZ32745 | | | | |
| | 94 AAZ32744 94 AAZ32743 94 AAZ32742 94 AAZ32741 95 | | | | |
| | AAZ32740 96 AAZ32739 97 AAZ32738 97 AAZ32737 97 AAZ32736 | | | | |
| | 97 AAZ32735 97 AAZ32734 96 AAZ32733 96 AAZ32732 97 | | | | |
| | AAZ32731 97 AAZ32730 97 AAZ32729 97 ABC49716 111 | | | | |
| | ABA43046 36 ABA43045 36 ABA43044 36 ABA43043 36 | | | | |
| | ABA43042 36 ABA43041 36 ABA43040 36 ABA43039 36 | | | | |
| | ABA43038 36 ABA43037 36 ABA43036 36 ABA43035 36 | | | | |
| | ABA43034 36 ABA43033 37 ABA43032 37 ABA43031 36 | | | | |
| | ABA43030 37 ABA43029 37 ABA43028 36 ABA43027 36 | | | | |
| | ABA43026 36 ABA43025 36 ABA43024 36 ABA43023 36 | | | | |
| | ABA43022 36 ABA43021 36 ABA43020 36 ABA43019 36 | | | | |
| | ABA43018 36 ABA43017 36 ABA43016 36 ABA43015 36 | | | | |
| | ABA43014 36 ABA43013 36 ABA43012 36 ABA43011 36 | | | | |
| | ABA43010 36 ABA43009 34 ABA43008 36 ABA43007 36 | | | | |
| | ABA43006 36 ABA43005 36 ABA43004 36 ABA43003 36 | | | | |
| | ABA54595 97 ABA54594 97 ABA54593 97 ABA54592 97 | | | | |
| | ABA54591 97 ABA54590 97 ABA54589 97 ABA54588 97 | | | | |
| | ABA54587 97 ABA54586 97 ABA54585 98 ABA54584 97 | | | | |
| | ABA54583 105 ABA54582 97 ABA54581 93 ABA54580 97 | | | | |
| | ABA54579 97 ABA54578 97 ABA54577 97 ABA54576 97 | | | | |
| | ABA54575 97 AAY54162 97 | | | | |
| 2006 | ABI81406 32 ABI81405 32 ABI81404 32 ABI81403 32 ABI81402 32 | 312 | 6.0 | 1.3 | low p < 0.001, |
| | ABI81401 32 ABI81400 32 ABI81399 32 ABI81398 32 ABI81397 32 | | | | prev p < 0.001 |
| | ABI81396 32 ABI81395 32 ABI81394 32 ABI81393 32 ABI81392 32 | | | | |
| | ABI81391 32 ABI81390 32 ABI81389 32 ABI81388 32 ABI81387 32 | | | | |
| | ABI81386 32 ABI81385 32 ABI81384 32 ABI81383 32 ABI81382 32 | | | | |
| | ABI81381 32 ABI81380 32 ABI81379 32 ABI81378 32 ABI81377 32 | | | | |
| | ABI81376 32 ABI81375 32 ABI81374 32 ABI81373 32 ABI81372 32 | | | | |
| | ABI81371 32 ABI81370 32 ABI81369 32 ABI81368 32 ABI81367 32 | | | | |
| | ABI81366 32 ABI81365 32 ABI81364 32 ABI81363 32 ABI81362 32 | | | | |
| | ABI81361 32 ABI81360 32 ABI81359 32 ABI81358 32 ABI81357 32 | | | | |
| | ABI81356 32 ABI81355 32 ABI81354 32 ABI81353 32 ABI81351 32 | | | | |
| | ABI81350 32 ABI81349 32 ABI81348 32 ABI81347 32 ABI81346 32 | | | | |
| | ABI81345 32 ABI81344 32 ABI81343 32 ABI81342 32 ABI81341 32 | | | | |
| | ABI81340 32 ABI81339 32 ABI81338 32 ABI81337 32 ABI81336 32 | | | | |
| | ABI81335 32 ABI81334 32 ABI81333 32 ABI81332 32 ABI81331 32 | | | | |
| | ABI81330 32 ABI81329 32 ABI81328 32 ABI81327 32 ABI81326 32 | | | | |
| | ABI81325 32 ABI81324 32 ABI81323 32 ABI81322 32 ABI81321 34 | | | | |
| | ABI81320 32 ABI81319 32 ABI81318 32 ABI81317 32 ABI81316 32 | | | | |
| | ABI81315 32 ABI81314 32 ABI81313 32 ABI81312 32 ABI81311 32 | | | | |
| | ABI81310 32 ABI81309 32 ABI81308 32 ABI81307 32 ABI81306 32 | | | | |
| | ABI81305 32 ABI81304 32 ABI81303 32 ABI81302 32 ABI81301 32 | | | | |
| | ABI81300 32 ABI81299 32 ABI81298 32 ABI81297 32 ABI81296 32 | | | | |
| | ABI81295 32 ABI81294 32 ABI81293 32 ABI81292 32 ABI81291 32 | | | | |
| | ABI81290 32 ABI81289 32 ABI81288 32 ABI81287 32 ABI81286 32 | | | | |
| | ABI81285 32 ABI81284 32 ABI81283 32 ABI81282 32 ABI81281 32 | | | | |
| | ABI81280 32 ABI81279 32 ABI81278 32 ABI81277 32 ABI81276 32 | | | | |
| | ABI81275 32 ABI81274 32 ABI81273 32 ABI81272 32 ABI81271 32 | | | | |
| | ABI81270 32 ABI81269 32 ABI81268 32 ABI81267 32 ABI81266 32 | | | | |
| | ABI81265 32 ABI81264 32 ABI81263 32 ABI81262 32 ABI81261 32 | | | | |
| | ABI81260 32 ABI81259 32 ABI81258 32 ABI81257 32 ABI81256 32 | | | | |
| | ABI81255 32 ABI81254 32 ABI81253 32 ABI81252 32 ABI81251 32 | | | | |
| | ABI81250 32 ABI81249 32 ABI81248 32 ABI81247 32 ABI81246 32 | | | | |
| | ABI81245 32 ABI81244 32 ABI81243 32 ABI81242 32 ABI81241 32 | | | | |
| | ABI81240 32 ABI81239 32 ABI81238 32 ABI81237 32 ABI81236 32 | | | | |
| | ABI81235 32 ABI81234 32 ABI81233 32 ABI81232 32 ABI81231 32 | | | | |
| | ABI81230 32 ABI81229 32 ABI81228 32 ABJ90133 32 ABJ90132 32 | | | | |
| | ABJ90131 32 ABJ90130 32 ABJ90129 32 ABJ90128 32 ABJ90127 32 | | | | |
| | ABJ90126 32 ABJ90125 32 ABJ90124 32 ABJ90123 32 ABJ90122 32 | | | | |
| | ABJ90121 32 ABJ90120 32 ABJ90119 32 ABJ90118 32 ABJ90117 32 | | | | |
| | ABJ90116 32 ABJ90115 32 ABJ90114 32 ABJ90113 32 ABJ90112 32 | | | | |
| | ABJ90111 32 ABJ90110 32 ABJ90109 32 ABJ90108 32 ABJ90107 32 | | | | |
| | ABJ90106 32 ABJ90105 32 ABJ90104 32 ABJ90103 32 ABJ90102 32 | | | | |
| | ABJ90101 32 ABJ90100 32 ABJ90099 32 ABJ90098 32 ABJ90097 32 | | | | |
| | ABJ90096 32 ABJ90095 32 ABJ90094 32 ABJ90093 32 ABJ90092 32 | | | | |
| | ABJ90091 32 ABJ90090 32 ABJ90089 32 ABJ90088 32 ABJ90087 32 | | | | |
| | ABJ90086 32 ABJ90085 32 ABJ90084 32 ABJ90083 32 ABJ90082 32 | | | | |
| | ABJ90081 32 ABJ90080 32 ABJ90079 32 ABJ90078 32 ABJ90077 32 | | | | |
| | ABJ90076 32 ABJ90075 32 ABJ90074 32 ABJ90073 32 ABJ90072 32 | | | | |
| | ABJ90071 32 ABJ90070 32 ABJ90069 32 ABJ90068 32 ABJ90067 32 | | | | |
| | ABJ90066 32 CAL49454 98 ABI97486 99 ABG36517 36 ABG81344 | | | | |
| | 92 ABG81343 97 ABG81342 97 ABG81341 97 ABG81340 99 | | | | |
| | ABG76816 41 ABG76815 43 ABG76814 43 ABG76813 43 | | | | |
| | ABG76812 43 ABG76811 43 ABG76810 43 ABG76809 43 | | | | |
| | ABG76808 43 ABG76807 43 ABG76806 43 ABG76805 43 | | | | |
| | ABG76804 43 ABG76803 43 ABG76802 43 ABG76801 43 | | | | |
| | ABG76800 43 ABG76799 43 ABG76798 43 ABG76797 43 | | | | |
| | ABG76796 43 ABG76795 43 ABI26622 40 ABI26621 40 ABD19642 | | | | |
| | 97 ABD19641 97 ABD19640 97 ABD19513 97 ABD19512 96 | | | | |
| | ABD19511 97 ABD19510 97 ABD85083 98 ABD85082 93 | | | | |
| | ABD85081 97 ABD85080 97 ABD85078 97 ABD85077 97 | | | | |
| | ABD85076 97 ABD85075 97 ABD85074 99 ABD85073 97 | | | | |
| | ABD85072 99 ABD85070 97 ABD85069 96 ABD85068 97 | | | | |
| | ABD85067 97 ABD85066 95 ABD85065 97 ABD85064 97 | | | | |
| | ABD67762 97 ABD67761 97 ABD67760 97 ABD67759 97 | | | | |
| | ABD67758 97 ABD67757 97 | | | | |
| 2007 | ABR19639 111 ABV22897 97 ABU54838 97 ABU52997 98 | (Incomplete) 27 | (Incomplete) | (Incomplete) | (Incomplete) |
| | ABQ52692 97 ABO69610 36 ABO69609 36 ABO69608 36 | | 4.6 | 1.2 | low p < 0.001, |
| | ABO69607 36 ABO69606 36 ABO69605 36 ABO69604 36 | | | | prev p < 0.001 |
| | ABO69603 36 ABO69602 36 ABO69601 36 ABO69600 36 | | | | |
| | ABO69599 36 ABO69598 36 ABO69597 36 ABO69596 36 | | | | |
| | ABO69595 36 ABO69594 36 ABO69593 36 ABO69592 36 | | | | |
| | ABU41789 114 CAM91200 97 ABR10608 56 | | | | |
| 2008 | ABZ10682 21 ABZ10681 29 ABZ10680 29 ABZ10679 29 ABZ10678 | (Incomplete) 5 | (Incomplete) | (Incomplete) | (Incomplete) |
| | 29 | | 5.5 | 0.7 | low p < 0.002, |
| | | | | | prev p < 0.04 |

### Example 4

### Analysis of Replikin Count Cycles in Foot and Mouth Virus to Predict Increased Morbidity

All protein sequences from isolates taken between 1999 and 2008 that were publicly available at www.pubmed.com were analyzed for Replikin sequences and a mean annual Replikin Count was determined. The data are contained in Table 3 above and illustrated in FIG. 4. FIG. 4 illustrates cycling of mean annual Replikin Count in foot and mouth disease virus type O. The peaks in the cycles correlate with outbreaks in the U.K and the Netherlands in 2001-2002 and in the Middle East and Asia in 2008-2009. The cycles illustrated in FIG. 4 are detectable because of repeating conserved virus structures and continuity of the Replikin phenomenon through time. In a new cycle beginning in 2005, the highest Counts in ten years was observed (2007-2008), which was followed by severe FMDV outbreaks in 2008 and 2009 in the Middle East, Africa, India, China, and other Asian countries.

FIG. 4 shows that the annual Replikin Counts (Mean and Standard Deviation (SD)) occurred with two rising portions and a decreasing portion. The first rising portion followed by the first decreasing portion occurred from, 1999-2005 and the second rising portion occurred in 2005-2008. Increases in Replikin Counts provided advance warning signals with p<0.001 prior to the 2001-2002 and 2008-2009 severe outbreaks.

To provide the data in FIG. 4, Replikin peptides were identified and counted automatically in sequences available at www.pubmed.com using the ReplikinsForecast™ software (Replikin LLC Boston, Mass.) designed to analyze protein sequences of any organism. Statistical analysis was likewise accomplished using the software. When the history of each Replikin structure in the virus was tracked for its occurrence in each virus specimen in each of the years for which virus sequence data was published, conservation of certain Replikin structures was observed over decades. The structure of these conserved Replikin peptides is the basis of synthetic Replikin vaccines for FMDV.

The following Replikin peptide sequences were identified for vaccines: HKQKIIAPAK (SEQ ID NO: 17) and HKQKIVAPVK (SEQ ID NO: 18). These sequences have been observed to be conserved in the Replikin cycles illustrated in FIG. 4 and, as taught by the invention, are vaccines for predicted outbreaks of foot and mouth disease virus.

The two above-listed conserved Replikin peptides have been identified and tracked annually in publicly available sequences in foot and mouth disease virus at www.pubmed.com from 1934 through 2008. The sequence HKQKIIAPAK (SEQ ID NO: 17) is observed to be conserved 100% of the time in the publicly available sequences from isolates from 1934 through 2008. The sequence HKQKIVAPVK (SEQ ID NO: 18) is observed also to be conserved in 100% of isolates from 1934 through 2007 with the exception of two substitutions, namely a valine at residue 6 in the peptide and a valine at residue 9 in the peptide.

Table 8 provides the accession numbers at www.pubmed.com wherein sequence HKQKIIAPAK (SEQ ID NO: 17) and HKQKIVAPVK (SEQ ID NO: 18) were conserved over time. The residue at which the peptide begins in the sequence disclosed in the accession number is noted.

TABLE 8
FMDV Conserved Sequences

| | Accession Numbers in which | Accession Numbers in which |
|---|---|---|
| | hkqkiiapak (SEQ ID NO: 17) are | hkqkivapvk (SEQ ID NO: 18) are |
| Year | conserved | conserved |
| 1934 | ACC63172 position 201, ACC63171 position | ACC63139 position 201, ACC63138 position 201, |
| | 201, ACC63169 position 201, ACC63168 | ACC63137 position 201, ACC63130 position |
| | position 201, ACC63167 position 201, | 201, ACC63129 position 201, ACC63128 |
| | ACC63165 position 200, ACC63164 position | position 201, ACC63127 position 201, |
| | 201, ACC63162 position 200, ACC63160 | ACC63133 position 201, ACC63132 position 201, |
| | position 201, ACC63159 position 201, | ACC63131 position 201 |
| | ACC63158 position 200, ACC63155 position | |
| | 200, ACC63154 position 201, ACC63153 | |
| | position 201, ACC63152 position 201, | |
| | ACC63151 position 200, ACC63150 position | |
| | 200, ACC63149 position 201, ACC63148 | |
| | position 201, ACC63147 position 200, | |
| | ACC63146 position 201, ACC63145 position | |
| | 200, ACC63144 position 200, ACC63143 | |
| | position 200, ACC63142 position 201, | |
| | ACC63140 position 201. | |
| 1955 | CAB62583 position 926. | |
| 1958 | CAA10475 position 131. | |
| 1962 | | CAC22210 position 202, AAP81678 position 153, |
| | | AAP81677 position 153, AAP81676 position 153, |
| | | AAP81675 position 153, AAP81674 position 153, |
| | | ABA46701 position 201, ABA46700 position 201, |
| | | ABA46699 position 201, ABA46698 position 201, |
| | | ABA46697 position 201, ABA46696 position 201, |
| | | ABA46695 position 201, ABA46693 position 201, |
| | | ABA46692 position 201, ABA46691 position 201, |
| | | ABA46690 position 201, ABA46689 position 201, |
| | | ABA46688 position 201, ABA46687 position 201, |
| | | ABA46686 position 201, ABA46685 position 201, |
| | | ABA46684 position 201, ABA46683 position 201, |
| | | ABA46682 position 201, ABA46681 position 201, |
| | | ABA46679 position 201, ABA46678 position 201, |
| | | ABA46677 position 201, ABA46675 position 201, |
| | | ABA46674 position 201, ABA46673 position 201, |
| | | ABA46672 position 201, ABA46671 position 201, |
| | | ABA46670 position 201, ABA46669 position 201, |
| | | ABA46668 position 201, ABA46666 position 201, |
| | | ABA46664 position 201, ABA46663 position 201, |
| | | ABA46662 position 201, ABA46661 position 201, |
| | | ABA46660 position 201, ABA46659 position 201, |
| | | ABA46658 position 201, ABA46657 position 201, |
| | | ABA46655 position 201, ABA46654 position 201, |
| | | ABA46653 position 201, ABA46652 position 201, |
| | | ABA46651 position 201, ABA46650 position 201, |
| | | ABA46649 position 201, ABA46648 position 201, |
| | | ABA46647 position 201, ABA46644 position 201, |
| | | ABA46643 position 201, ABA46642 position 201, |
| | | ABA46641 position 201, ABA46640 position 201, |
| | | ABA46639 position 201, ABA46638 position 201, |
| | | ABA46637 position 201, ABA46614 position 201, |
| | | ABA46613 position 201, ABA46612 position 201, |
| | | ABA46611 position 201, ABA46610 position 201, |
| | | ABA46609 position 201, ABA46606 position 201, |
| | | ABA46605 position 201, ABA46604 position 201, |
| | | ABA46603 position 201, ABA46602 position 201, |
| | | ABA46601 position 201, ABA46600 position 201, |
| | | ABA46597 position 201, ABA46596 position 201, |
| | | ABA46594 position 201, ABA46591 position 201, |
| | | ABA46590 position 201, ABA46589 position 201, |
| | | ABA46588 position 201, ABA46586 position 201, |
| | | ABA46585 position 201, ABA46583 position 201, |
| | | ABA46582 position 201, ABA46581 position 201, |
| | | ABA46580 position 201, ABA46579 position 201, |
| | | ABA46578 position 201, ABA46576 position 201, |
| | | ABA46574 position 201, ABA46573 position 201, |
| | | ABA46571 position 201, ABA46570 position 201, |
| | | ABA46569 position 201, ABA46568 position 201, |
| | | ABA46566 position 201, ABA46565 position 201, |
| | | ABA46563 position 201, ABA46561 position 201, |
| | | ABA46560 position 201, ABA46542 position 201, |
| | | ABA46541 position 201, ABA46539 position 201, |
| | | ABA46538 position 201, ABA46537 position 201, |
| | | ABA46536 position 201, ABA46535 position 201, |
| | | ABA46534 position 201, ABA46533 position 201, |
| | | ABA46532 position 201, ABA46531 position 201, |
| | | ABA46559 position 201, ABA46540 position 201, |
| 1964 | CAB62582 position 640 | |
| 1968 | CAC48168 position 201. | |
| 1969 | | CAB62584 position 724. |
| 1971 | CAC48169 position 201. | |
| 1972 | ABL75440 position 40, ABL75439 position 43, | CAC22304 position 202 |
| | ABL75437 position 43, ABL75435 position 43, | |
| | ABL75434 position 43, ABL75433 position 43, | |
| | ABL75432 position 43, ABL75431 position 43, | |
| | ABL75427 position 43, ABL75424 position 43, | |
| | ABL75423 position 43, ABL75422 position 43, | |
| | ABL75421 position 43, ABP82766 position 200, | |
| | ABP82765 position 200, ABP82764 position | |
| | 201, ABP82763 position 201, ABP82762 | |
| | position 201, ABP82759 position 200, | |
| | ABP82757 position 200, ABP82756 position | |
| | 201, ABP82755 position 201, ABP82754 | |
| | position 201, ABP82753 position 201, | |
| | ABP82752 position 201, ABP82751 position | |
| | 201, ABP82750 position 201, ABP82749 | |
| | position 201, ABP82748 position 201, | |
| | ABP82747 position 201, ABP82746 position | |
| | 201, ABP82744 position 201. | |
| 1974 | | CAC22211 position 202, AAK69575 position 153, |
| | | AAR85362 position 153, AAR22955 position 153, |
| | | AAR22953 position 153 |
| 1975 | | AAK69576 position 153, CAC20174 position 201, |
| | | AAR85363 position 153, AAG35653 position |
| | | 724 |
| 1976 | CAC34727 position 201. | AAR22952 position 153, AAR22933 position 153, |
| | | AAR22932 position 153. |
| 1977 | AAD26458 position 58, AAD26457 position 58, | AAR22963 position 153, AAR22950 position 153, |
| | AAD26456 position 58, AAD26455 position | CAC48179 position 201. |
| | 48, AAD26454 position 58, AAD26452 | |
| | position 58, AAD26451 position 58, | |
| | AAD26450 position 58, AAD26449 position 58, | |
| | AAD26448 position 52, AAD26447 position | |
| | 48, AAD26446 position 58, AAD26445 | |
| | position 58, AAD26443 position 58, | |
| | AAD26442 position 58, AAD26438 position 58, | |
| | AAD26437 position 58, AAD26436 position | |
| | 58, AAD26435 position 58, AAD26434 | |
| | position 58, AAD26433 position 53, | |
| | AAD26432 position 58, AAF75833 position | |
| | 725 | |
| 1978 | | ABA46745 position 201, ABA46744 position 201, |
| | | ABA46743 position 201, ABA46742 position 201, |
| | | ABA46740 position 201, AAR22930 position 153. |
| 1979 | | CAC22173 position 43, AAQ88330 position 153, |
| | | AAQ88328 position 153, AAQ88327 position 153, |
| | | AAQ88325 position 153, AAQ88324 position |
| | | 153, AAQ88323 position 153, AAQ88322 |
| | | position 153, AAQ88321 position 153, |
| | | AAQ88320 position 153, AAQ88319 position 153, |
| | | AAQ88318 position 153, AAQ88317 position |
| | | 153, AAQ88316 position 153, AAQ88315 |
| | | position 153, AAQ88314 position 153, |
| | | AAQ88313 position 153, AAQ88312 position 153, |
| | | AAG28368 position 43, AAG28367 position 43, |
| | | AAG28366 position 43, AAG28362 position 43, |
| | | AAG28357 position 43, AAG28356 position 43, |
| | | AAG28355 position 43, AAG28354 position 43, |
| | | AAG28353 position 43, AAG28352 position 43, |
| | | AAG28348 position 43. |
| 1980 | | AAR22962 position 153, AAR22959 position 153, |
| | | AAR22941 position 153 |
| 1981 | CAC27325 position 201. | AAR22951 position 153 |
| 1982 | AAA42596 position 190, P03309 position 190. | CAC20178 position 201, AAZ31359 position 201, |
| | | AAZ31358 position 201, AAZ31357 position 201, |
| | | AAZ31356 position 201, AAZ31354 position 201, |
| | | AAZ31353 position 201, AAZ31352 position 201, |
| | | AAZ31350 position 201, AAZ31349 position 201, |
| | | AAZ31348 position 201, AAZ31347 position 201, |
| | | AAZ31346 position 201, AAZ31345 position 201, |
| | | AAZ31344 position 201, AAZ31343 position 201, |
| | | AAZ31342 position 201. |
| 1983 | | AAR22960 position 153, AAR22938 position 153, |
| | | AAR22937 position 153 |
| 1984 | ABZ80842 position 201, CAA00589 position | |
| | 190. | |
| 1985 | AAA42601 position 201, AAA42598 position | CAC22326 position 90. |
| | 157, AAA42597 position 200, AAA42595 | |
| | position 198, AAA42594 position 201 | |
| 1986 | AAB93439 position 71, ABZ80846 position | AAR22954 position 153. |
| | 202, AAA42664 position 225. | |
| 1987 | AAB93449 position 93, AAB05766 position | AAK62003 position 43. |
| | 201, AAB05764 position 201, AAB05763 | |
| | position 201, AAB05762 position 201, | |
| | AAB93450 position 69, AAA42604 position | |
| | 183, AAA42614 position 75, AAA42603 | |
| | position 183, AAA42602 position 180, | |
| | CAC27328 position 201, CAC27326 position | |
| | 201. | |
| 1988 | | AAK69568 position 153, AAK69567 position 153. |
| 1989 | | CAC22174 position 90, AAR22961 position 153, |
| | | AAK62024 position 69. |
| 1990 | CAC48172 position 201, CAC48170 position | CAC22178 position 43, CAC22327 position 58 |
| | 201. | |
| 1991 | AAA42666 position 708, CAC48173 position | CAC22175 position 43, CAC22328 position 62 |
| | 201. | |
| 1992 | CAC48176 position 201 | CAC22176 position 43, CAC22240 position 85, |
| | | CAC48182 position 201. |
| 1993 | | CAC22179 position 43, CAC40792 position 201, |
| | | CAC40789 position 201, CAC40796 position 102. |
| 1994 | | CAC22180 position 76, CAC22233 position 62, |
| | | CAC22227 position 60, CAC22215 position 47, |
| | | CAC22208 position 82, CAC22201 position 43, |
| | | CAC22167 position 43, AAK62012 position 43, |
| | | CAC40794 position 102, CAC40790 position 201, |
| | | CAC40795 position 102, CAC40797 position |
| | | 201. |
| 1995 | | CAC22231 position 152, CAC22216 position 44, |
| | | CAC22171 position 103, AAK62022 position 69 |
| 1996 | AAB05765 position 201. | CAC22194 position 127, CAC51235 position 201, |
| | | AAR22945 position 153, AAR22942 position 153, |
| | | AAK62005 position 69 |
| 1997 | | CAC51273 position 201, CAC51268 position 201, |
| | | CAC51249 position 201, CAC51236 position |
| | | 201, AAL05257 position 43, AAL05249 position |
| | | 43, AAL05248 position 85, AAL05247 position |
| | | 62, AAL05246 position 76, AAL05245 position |
| | | 43, AAL05243 position 56, AAL05242 position |
| | | 43, AAL05236 position 43, AAL05235 position |
| | | 65, AAL05234 position 43, AAL05233 position |
| | | 43, AAL05232 position 43, AAL05231 position |
| | | 43, AAL05230 position 43, AAL05229 position |
| | | 43, AAL05228 position 43, AAL05227 position |
| | | 85, AAL05226 position 43, AAL05225 position |
| | | 76, AAL05223 position 43, AAL05222 position |
| | | 43, AAL05221 position 43, AAL05220 position |
| | | 122, AAL05219 position 43, AAL05218 position |
| | | 52, AAL05217 position 43, AAL05216 position |
| | | 66, AAL05214 position 43, AAL05213 position |
| | | 93, AAL05211 position 58, AAL05207 position |
| | | 43, AAL05206 position 62, AAL05205 position |
| | | 67, AAL05196 position 64. |
| 1998 | AAL73360 position 113. | CAC22229 position 201, ABI16250 position 201, |
| | | ABI16249 position 201, ABI16248 position 201, |
| | | ABI16247 position 201, ABI16246 position 201, |
| | | ABI16245 position 201, ABI16244 position 201, |
| | | ABI16242 position 201, ABI16241 position 201, |
| | | ABI16240 position 201, ABI16239 position 201, |
| | | ABI16238 position 201, ABI16237 position 201, |
| | | ABI16236 position 201, ABI16235 position 201, |
| | | ABI16234 position 201, ABI16233 position 201, |
| | | ABI16232 position 201, ABI16231 position 201, |
| | | ABI16230 position 201, ABI16229 position 201, |
| | | ABI16228 position 201, ABI16227 position 201, |
| | | CAC51269 position 201, AAR85364 position 153, |
| | | AAR22957 position 153, AAL05256 position 43, |
| | | AAL05255 position 43, AAL05254 position 43, |
| | | AAL05253 position 43, AAL05250 position 43, |
| | | AAL05244 position 43, AAL05241 position 43, |
| | | AAL05240 position 43, AAL05238 position 43, |
| | | AAL05237 position 45, AAL05212 position 43. |
| 1999 | | CAC22228 position 100, CAC22200 position 100, |
| | | AAG43385 position 43, CAC51332 position 143, |
| | | CAC51270 position 175, CAC51255 position |
| | | 201, CAC51318 position 201, CAC51247 |
| | | position 201, CAC51246 position 201, |
| | | CAC51245 position 201, CAD62370 position 925, |
| | | CAD62369 position 925, CAC20187 position |
| | | 201, AAR22956 position 153, AAR22940 position |
| | | 153, ACD44908 position 201, ACD44906 |
| | | position 201, AAF06146 position 43, AAD41912 |
| | | position 81, AAD41131 position 81, AAL05251 |
| | | position 43, AAL05215 position 43, AAL05210 |
| | | position 43, AAL05209 position 43, AAL05208 |
| | | position 43, AAL05204 position 43, AAL05203 |
| | | position 45, AAL05202 position 43, AAL05201 |
| | | position 43, AAL05200 position 43, AAL05199 |
| | | position 43, AAL05198 position 43, AAL05197 |
| | | position 70, AAL05195 position 59, AAL05194 |
| | | position 58, AAL05193 position 43, AAL05192 |
| | | position 43, AAL05191 position 43. |
| 2000 | ABF18566 position 43, ABF18562 position 43, | CAC22209 position 201, AAL09392 position 153, |
| | ABF18557 position 43, ABF18555 position 43, | AAL09391 position 153, AAK69397 position 153, |
| | ABF18553 position 43, ABF18552 position 43, | ABF18551 position 43, ABF18550 position 43, |
| | ABL60850 position 201, ABL60849 position | ABF18549 position 43, ABF18548 position 43, |
| | 201, ABL60848 position 201, ABL60847 | CAC51275 position 201, CAC51271 position 201, |
| | position 201, ABL60845 position 201, | CAC51267 position 201, CAC51264 position |
| | ABL60844 position 201, ABL60843 position | 201, CAC51263 position 201, CAC51261 |
| | 201, ABL60842 position 201, ABL60841 | position 201, CAC51258 position 201, |
| | position 201, ABL60840 position 201, | CAC51257 position 201, BAC06475 position 925, |
| | ABL60839 position 201, ABL60838 position | AAG27038 position 153, AAG27037 position |
| | 201, ABL60837 position 201, ABL60836 | 153, AAR22931 position 153, ACD44909 |
| | position 201, ABL60835 position 201, | position 201, ABA46733 position 201, ABA46732 |
| | ABL60834 position 201, ABL60833 position | position 201, ABA46731 position 201, ABA46730 |
| | 201, ABL60832 position 201. | position 201, ABA46729 position 201, ABA46728 |
| | | position 201, ABA46727 position 201, ABA46726 |
| | | position 201, ABA46725 position 201, ABA46724 |
| | | position 201, ABA46723 position 201, ABA46722 |
| | | position 201, ABA46721 position 201, ABA46720 |
| | | position 201, ABA46719 position 201, ABA46717 |
| | | position 200, ABA46716 position 201, ABA46715 |
| | | position 201, ABA46714 position 201, ABA46713 |
| | | position 201, ABA46712 position 201, ABA46711 |
| | | position 201, ABA46709 position 201, ABA46708 |
| | | position 201, ABA46706 position 201, ABA46705 |
| | | position 201, ABA46704 position 201, BAB18050 |
| | | position 201, ABV53920 position 201. |
| 2001 | ABY75530 position 78, ABY75529 position 78, | CAD62373 position 925, AAK92375 position 925, |
| | ABY75528 position 78, ABY75527 position | ACD44910 position 201, CAC35464 position |
| | 76, ABY75526 position 78, ABY75524 | 201, CAC35463 position 201, CAC35462 |
| | position 78, ABY75523 position 78, | position 201, CAC35461 position 201, |
| | ABY75522 position 78, ABY75521 position 78, | CAG23917 position 925, CAC86575 position 925 |
| | ABY75520 position 78, ABY75519 position | |
| | 78, ABZ80836 position 201. | |
| 2002 | ABZ80844 position 201, ABZ80835 position | AAR07959 position 153, AAM62134 position 201. |
| | 201. | |
| 2003 | ABZ80845 position 201, AAR00255 position | AAQ93493 position 925, AAR07963 position 153, |
| | 80, ABR13023 position 201, ABR13022 | AAR07962 position 153, AAR07961 position 153, |
| | position 201, ABR13021 position 201, | AAR07960 position 153, AAR07965 position 153, |
| | ABR13020 position 201. | ACD44915 position 201, ACD44914 position |
| | | 201, ACD44913 position 201, ACD44912 |
| | | position 201, ACD44911 position 201, |
| | | ACD44903 position 191, ACD44902 position 188, |
| | | ACD44898 position 192, ACD44897 position |
| | | 187, AAR07964 position 153, ABR13026 position |
| | | 201, ABR13025 position 201, ABR13024 position |
| | | 201 |
| 2005 | AAY56402 position 81, CAJ51050 position 201, | ABD14417 position 201, ABC55721 position 43, |
| | CAJ51049 position 201, CAJ51047 position | CAJ51080 position 201, CAJ51079 position 201, |
| | 201, CAJ51046 position 201, CAJ51045 | CAJ51078 position 201, CAJ51077 position 201, |
| | position 201, CAJ51043 position 201, | CAJ51076 position 201, CAJ51075 position 201. |
| | CAJ51042 position 201, CAJ51041 position | |
| | 201, CAJ51040 position 201, CAJ51039 | |
| | position 201. | |
| 2006 | ACD44924 position 200, ACD44923 position | ACD44919 position 201, ACD44916 position 201, |
| | 200, ACD44922 position 200, ABG77560 | ABG77563 position 197, ABG77564 position 30 |
| | position 219, ABG77557 position 126. | |
| 2007 | ABR18732 position 185, ABN70732 position | ABY75534 position 286, ABY75533 position 97 |
| | 171, ABN70731 position 217. | |
| 2008 | ACI96104 position 201 | |

### Example 5

### Analysis of Replikin Count Cycles in West Nile Virus to Predict Entry into Geographical Regions

As discussed above in Example 3, Applicants analyzed the Replikin concentration of West Nile virus envelope protein isolates publicly available in accession numbers of www.pubmed.com. As seen in FIG. 3, the cycles of mean annual Replikin concentration in the envelope proteins of the isolates are related to cycles of morbidity in the United States. Additionally, the cycles of mean annual Replikin concentration are related to step-wise geographical expansion into the United States from the first known infection of West Nile virus in the state of New York in 1998.

For example, as mean annual Replikin concentration increased between 2000 and 2003, West Nile virus morbidity expanded initially from New York and certain contiguous states in 2000, to the Northeast and Southeast in 2001, to most states except the Mountain states and Northwest in 2002, and to all states but the Northwest in 2003. See, e.g., annual maps available from the CDC at http://www.cdc.gov/ncidod/dvbid/westnile/surv&control.htm#maps. When the mean annual Replikin concentration began to fall in 2004, West Nile Virus was present in all continental U.S. states but with a much lower rate of morbidity. In 2005, West Nile virus infections were observed to retreat from certain parts of the U.S. and infections were not observed in Washington State, northern New England, or West Virginia. However, as annual mean Replikin concentrations began to increase again in 2006, West Nile virus morbidity again spread to all states except northern New England.

A review of the progression of West Nile virus across the United States from its first observation in New York reveals that monitoring changes in Replikin concentration provides evidence of geographic expansion of West Nile Virus. An aspect of the invention, therefore, is the prediction of an expansion into a geographic region or contraction from a geographic region based on a determination of the progression of mean annual Replikin concentrations in a graph of a cycle or series of cycles of Replikin concentration including observed step-wise cycles. For example, a peak in Replikin concentration in a cycle of Replikin concentration of a plurality of isolates from a given region provides evidence of expansion beyond the geographical area of that region into other contiguous or nearby geographical areas. Furthermore a second, still higher, peak provides even greater evidence of a pathogen that is poised for expansion.

### Example 6

### Analysis of Replikin Count Cycles in Malaria to Predict Entry into Geographical Regions

The phenomenon of geographical expansion also applies to malaria and other pathogens. Analysis of the Replikin concentration of a Replikin Peak Gene, histidine-rich protein, or ATP-ase of P. falciparum demonstrates that Replikin concentration cycles may provide a prediction of an expansion of P. falciparum mortality and/or morbidity. For example, if a Replikin concentration cycle based on isolates from a particular region demonstrates a prolonged rise in mean annual Replikin Count or a peak following a rise in mean annual Replikin Count, the significant rise or peak predicts an expansion of the mortality rate or morbidity rate of that isolate into contiguous or nearby regions that until the significant rise or peak in Replikin Count did not experience the mortality rate or morbidity rate of the particular region.

For example, a cycle of Replikin concentration is established in the Sahel region of Africa with two peaks at years 2 and 7. The second peak at year 7 is significantly higher than the first peak at year 2 with a p value of 0.01. The Sahel region between years 0 and 7 has experienced a higher rate of mortality than more southerly regions. Based on the higher peak at year 7, it is predicted that the mortality from malaria will increase in the region contiguous to the south of the Sahel. A plurality of Replikin sequences are isolated from year 7 isolates. Replikins that have been conserved between years 0 and 7 are selected as vaccines for malaria in the Sahel and contiguous regions to the south. Replikins that are new in year 7 are likewise selected as vaccines. A mixture of these Replikin sequences is combined with a pharmaceutically acceptable carrier and/or adjuvant and administered to a subject to produce an immune response to treat and/or protect against malaria predicted to have a higher mortality rate following the dry season in year 8 in the Sahel and in its contiguous regions to the south.

### Example 7

### Replikin Count Virus Expansion Index in Same and Related Influenza Strains over Time

Applicants analyzed all amino acid sequences of the pB1 gene area of isolates of H5N1 strains of influenza virus publicly available at www.pubmed.com for specimens isolated between 2004 and 2008. Isolates were grouped by species of bird within countries for each year in which sequences were available.

The concentration of continuous and overlapping Replikin peptides in the pB1 gene area was determined for each isolate (the Replikin Count of the Replikin Peak Gene). Within each year in each country a mean Replikin Count with standard deviation was determined. China was found to have the largest number of isolates for each year from 2004 to 2008 and the mean Replikin Count (with standard deviation) of all H5N1 isolates from chicken in China in each year was chosen as a control against which other Replikin Counts would be determined (China was chosen as a control because of a limited variability in Replikin Count among a very large number of isolates available for analysis).

The Replikin Count for each individual isolate in a given country in a given year was compared to one standard deviation from the mean Replikin Count for all isolates from chicken in China in that year. Within each country, the number of Replikin Counts greater than one standard deviation of the mean and the number of Replikin Counts less than one standard deviation of the mean were determined. For each country in each year, the percent of Replikin Counts greater than one standard deviation of the mean was then divided by the percent of Replikin Counts less than one standard deviation of the mean to provide a ratio, or Replikin Count Virus Expansion (RCVE) Index. In countries having an RCVE Index of greater than one, an expansion of H5N1 was predicted for the following year or years. In countries having a RCVE Index of less than one, a contraction or viral failure was predicted for the following year or years.

Five sets of RCVE Indices are calculated and reported below as examples for the ordinary skilled artisan. The ordinary skilled artisan will understand how to repeat the predictive methods for data from any region, time, or species and will understand from the disclosure herein how to practice methods of prevention, mitigation, and treatment for outbreaks predicted by the RCVE Indices including therapeutic compounds identified in isolates predicted to be expanding in population.

In Tables 9-13 below, individual Replikin Counts that are above the reported standard deviation of the mean of the control are bolded. Individual Replikin Counts that are below the reported standard deviation of the mean of the control are italicized and bolded. The RCVE Index ratio is reported for each group of isolates as compared to the control.

In Table 9, Replikin Counts for individual H5N1 isolates from swans in China for 2004 are compared to a control of the annual mean Replikin Count for all chicken H5N1 isolates from China in 2004.

TABLE 9
H5N1 Replikin Counts 2004

| | Control |
|---|---|
| | (all H5N1 isolates |
| Individual Swans (China) | chickens in China) |
| 2.0, 2.4, 2.4, 2.0, 2.4, 2.0, 3.8, 2.0 | |
| Mean Annual RC = 2.3 | Mean Annual RC = 2.3 ± SD 1.1 |
| | no. of isolates = 533 |
| percent of isolates above | |
| (Mean + SD) = 12.5 | |
| percent of isolated below | |
| (Mean − SD) = 0 (equals 1 if in | |
| denominator of RCVE) | |
| RCVE Index = 12.5/1 = 12.5 | |

The RCVE Index for swans in China in 2004 is 12.5/0. Because zero is set as 1 when it is in the denominator, the index returns a ratio of 12.5, which predicts an expanding population. This predicted expansion is seen below in Table 11 in an expanding population in swans in China in 2006.

In Table 10, Replikin Counts for individual isolates from swans in Mongolia, Russia, and Japan in 2005 are compared to a control of the annual mean Replikin Count for all H5N1 chicken isolates from China in 2005.

TABLE 10
H5N1 Replikin Counts 2005

| | | | Control |
|---|---|---|---|
| Mongolia | Russia | Japan | (all H5N1 isolates from |
| (Individual Swans) | (Individual Swans) | (Individual Swans) | chickens in China) |
| 3.6, 3.7, 3.7, 3.7, 3.7, | 3.3, 2.0, 3.3, 2.0, 3.3, | 0 | Mean Annual RC = 2.5 ± |
| 2.4, 3.7, [symbol] , 2.0, 1.7, | 2.0, 1.8 | | SD 1.0 |
| 3.7, 3.1, 1.7, 1.8, 7.1, | | | no. of isolates = 362 |
| [symbol] , 3.1, 7.1, 2.1, 3.1, | | | |
| [symbol] , [symbol] , [symbol] , [symbol] , 2.3, | | | |
| 1.8, 1.8, 1.7, [symbol] | | | |
| percent of isolates | percent of isolates | percent of isolates | |
| above (Mean + SD) = | above (Mean + SD) = 0 | above (Mean + SD) = 0 | |
| 9/29 = 31% | percent of isolated | percent of isolated | |
| percent of isolated | below (Mean − SD) = 0 | below (Mean − SD) = 0 | |
| below (Mean − SD) = | | | |
| 7/29 = 24.1% | | | |
| RCVE Index = | No RCVE Index | No RCVE Index | |
| 31.0%/24.1% = 1.3 | | | |

The RCVE Index for swans in Mongolia in 2005 is 1.3, which predicts an expansion of the H5N1 population in Mongolia, because the RCVE Index is greater than 1. This predicted expansion from Mongolia is seen below in European countries, such as Sweden and Denmark, known to be in the flight path for swans and other birds from Mongolia.

In Table 11, Replikin Counts for individual isolates from a variety of bird species in eight different countries are compared to a control of the annual mean Replikin Count for all H5N1 chicken isolates from China in 2006. In Denmark, duck, swan, and falcon isolates are reported. In Czech Republic, turkey and falcon isolates are reported. All other non-control isolates are from swans.

TABLE 11
H5N1 Replikin Counts 2006

| | | | | | | | | Control |
|---|---|---|---|---|---|---|---|---|
| | | | | | | | | (all H5N1 |
| | | | | | | | | isolates |
| | | | | | | | | from |
| Sweden | Denmark | Germany | Slovenia | Scotland | Czech | Mongolia | China | chicken |
| (Swans) | (Duck)(Swan)(Falc) | (Swans) | (Swans) | (Swans) | (Turk)(Falc) | (Swans) | (Swans) | in China) |
| 2.0 | 2.4 | 2.4 | 3.8 | 3.6 | 4.3 | 3.6 | 3.7 | Mean |
| 2.5 | 2.4 | 3.6 | 2.4 | 2.4 | 4.3 | 2.4 | 2.0 | Annual |
| 3.4 | 3.6, 3.7 | 2.0 | 2.4 | | 4.3 | 2.0 | 3.7 | RC = |
| 3.4 | 3.6, 3.7 | 2.0 | 22.2 | | 4.3 | [symbol] | 2.0 | 2.7 ± |
| 3.4 | 3.9 | 2.4 | 17.8 | | 2.4, 2.2 | 17.8 | 2.5 | SD 0.8 |
| 2.5 | 3.9 | 2.0 | | | 2.4, 2.2 | 3.7 | 3.7 | no. of |
| 17.8 | 2.4 | 2.4 | | | 2.4, 2.2 | [symbol] | 2.0 | isolates = |
| 3.9 | 2.4 | 2.0 | | | 2.4, 2.2 | [symbol] | 2.5 | 576 |
| [symbol] | 2.0, 2.1 | | | | | 2.1 | 2.0 | |
| [symbol] | 2.4 | | | | | [symbol] | 2.5 | |
| [symbol] | 2.4, 2.0, 2.0 | | | | | 2.4 | | |
| [symbol] | [symbol] | | | | | [symbol] | | |
| | [symbol] | | | | | [symbol] | | |
| | [symbol] | | | | | | | |
| | [symbol] | | | | | | | |
| RCVE | RCVE | RCVE | RCVE | RCVE | RCVE | RCVE | RCVE | |
| Index = | Index = | Index = | Index = | Index = | Index = | Index = | Index = | |
| 16.7/33.3 = | 25/33.3 = | 12.5 | 60 | 50 | 33.3 | 23/46 = | 30 | |
| 0.50 | 0.75 | | | | | 0.50 | | |

The RCVE Index predicts expansion in Germany, Slovenia, Scotland, Czech Republic, and China. The Index predicts contraction or failure in Sweden, Denmark, and Mongolia. It is noteworthy that the index predicts contraction or failure of the H5N1 influenza population in swans in Mongolia in 2006 while in 2005 the index of 1.3 predicted expansion. In 2007, as predicted in 2006, no H5N1 isolates were reported in Mongolia. See Table 12 below.

In Table 12, Replikin Counts for individual isolates from swans in Japan for 2007 are compared to a control of the annual mean Replikin Count for all chicken H5N1 isolates from China in 2007.

TABLE 12
H5N1 Replikin Counts 2007

| | Control |
|---|---|
| | (all H5N1 isolates |
| Individual Swans (Japan) | chicken in China) |
| 2.0, 4.2, 3.8, 16.7 | |
| Mean Annual RC = 6.7 | Mean Annual RC = 2.7 ± SD 0.8 |
| | no. of isolates = 112 |
| percent of isolates above | |
| (Mean + SD) = 50 | |
| percent of isolated below | |
| (Mean − SD) = 0 (equals 1 if in | |
| denominator of RCVE) | |
| RCVE Index = 50/1 = 50 | |

The RCVE Index for swans in Japan in 2007 is 50/0. Because zero is set as 1 when it is in the denominator, the index returns a ratio of 50, which predicts an expanding population. So despite a small sample size, the index predicts expansion, which is seen below in Table 13 in an expanding population in swans in Japan.

In Table 13, Replikin Counts for individual isolates from swans in Japan for 2008 are compared to a control of the annual mean Replikin Count for all chicken H5N1 isolates from China in 2008. Only 3 isolates from chicken in 2008 were reported and available for analysis.

TABLE 13
H5N1 Replikin Counts 2008

| | Control |
|---|---|
| | (all H5N1 isolates |
| Individual Swans (Japan) | chicken in China) |
| 3.7, 3.7, 3.7, 3.7, 3.8, 3.8, 3.8, 3.8, 3.8, | |
| 3.8, 3.8, 3.8, 4.5, 17.8, 17.8, 17.8, | |
| 2.4, 1.8, 2.4, 1.8, 2.2, 2.1, 2.7, 2.4, 1.8, | |
| 1.2, 1.7, 1.7, 2.4, 1.8, 2.7, 2.1, 1.2, | |
| [symbol] , [symbol] , [symbol] , [symbol] , [symbol] , [symbol] , [symbol] , [symbol] , [symbol] | |
| Mean Annual RC = 6.7 | Mean Annual RC = 2.6 ± |
| | SD 0.9 no. of isolates = 3 |
| percent of isolates above | |
| (Mean + SD) = 38.1 | |
| percent of isolated below | |
| (Mean − SD) = 21.4 | |
| RCVE Index = 38.1/21.4 = 1.8 | |

The RCVE Index for swans in Japan in 2008 is 1.8, which predicts future expansion of influenza in swans in Japan.

The RCVE Indices as described above may be practiced by one of ordinary skill in the art as a measure of the current survival and expansion status or contracting/failing status of a population of pathogen engaged in an outbreak. The ordinary skilled artisan may isolate in silico the Replikin Peak Gene, may measure the Replikin Count of the Replikin Peak Gene, and may compare the Replikin Count data of related strains of virus in other geographic regions in the same and previous time periods to understand the severity of the outbreak, the direction of the outbreak, and the attendant risk to neighboring geographic regions. Like identifying and tracking a hurricane, the appreciable advantage to the ordinary skilled artisan is time to develop therapies and to institute public health measures known now or hereafter such as isolation and culling of poultry, vaccination, and other measures. The methods disclosed herein further provide the ordinary skilled artisan with time to manufacture the synthetic Replikin vaccines disclosed herein.

### Example 8

### Replikin Count Expansion Index in Same and Related Malarial Strains over Time

All publicly available sequences of the histidine rich protein gene of isolates P. falciparum from 2004 through 2008 are analyzed for Replikin concentration. Isolates are grouped by region.

Within each year and in each region a mean Replikin Count with standard deviation is determined. The region having the largest number of isolates or the least variability among Replikin Count in isolates (or both) for each year from 2004 to 2008 is chosen as a control against which other Replikin Counts are analyzed. The Replikin Count for each individual isolate in a given region in a given year is compared to one standard deviation from the mean Replikin Count for all isolates from the control region. Within each region, the number of Replikin Counts greater than one standard deviation of the mean and the number of Replikin Counts less than one standard deviation of the mean is determined. For each region in each year, the percent of Replikin Counts greater than one standard deviation of the mean is then divided by the percent of Replikin Counts less than one standard deviation of the mean to provide a ratio, or Replikin Count Expansion (RCE) Index. In regions having an RCE Index of greater than one, an expansion of malaria is predicted for the following year or years. In regions having an RCE Index of less than one, a contraction of malaria is predicted for the following year or years.

In regions wherein malaria is predicted to expand, a Replikin Peak Gene is identified in an isolate having a Replikin Count that is higher than the mean Replikin Count for the region. The Replikin Peak Gene and/or a Replikin peptide (or plurality of Replikin peptides) within the Replikin Peak Gene is selected as an immunogenic compound for diagnostic and/or therapeutic purposes. A vaccine against the expanding population is manufactured comprising the immunogenic compound. The vaccine is administered to mitigate the expanding malarial population.

### Example 9

### Replikin Count Virus Expansion Index in Same and Related Foot and Mouth Disease Virus Strains over Time

All publicly available sequences of the VP1 gene of isolates of Foot and Mouth Disease Virus Type O from 2004 through 2008 are analyzed for Replikin concentration. Isolates are grouped by region.

Within each year and in each region, a mean Replikin Count with standard deviation is determined. The region having the largest number of isolates or the least variability among Replikin Count in isolates (or both) for each year from 2000 to 2008 is chosen as a control against which other Replikin Counts are analyzed. The Replikin Count for each individual isolate in a given region in a given year is compared to one standard deviation from the mean Replikin Count for all isolates from the control region. Within each region, the number of Replikin Counts greater than one standard deviation of the mean and the number of Replikin Counts less than one standard deviation of the mean is determined. For each region in each year, the percent of Replikin Counts greater than one standard deviation of the mean is then divided by the percent of Replikin Counts less than one standard deviation of the mean to provide a ratio, or Replikin Count Virus Expansion (RCVE) Index. In regions having a RCVE Index of greater than one, an expansion of foot and mouth disease is predicted for the following year or years. In regions having a RCVE Index of less than one, a contraction of foot and mouth disease is predicted for the following year or years.

In regions wherein foot and mouth disease is predicted to expand, a Replikin Peak Gene is identified in an isolate having a Replikin Count that is higher than the mean Replikin Count for the region. The Replikin Peak Gene and/or a Replikin peptide (or plurality of Replikin peptides) within the Replikin Peak Gene is selected as an immunogenic compound for diagnostic and/or therapeutic purposes. A vaccine against the expanding population is manufactured comprising the immunogenic compound. The vaccine is administered to mitigate the expanding foot and mouth disease virus population.

### Example 10

### Synthetic Replikin Vaccines Block H5N1 in Chickens

A synthetic Replikin vaccine containing an approximately equal-parts-by-weight mixture of twelve H5N1 Replikin peptides was tested in chickens against a low pathogenic strain of H5N1 isolated from a black duck in North Carolina, USA. Low-Path H5N1 strains infect migratory birds and impair health and productivity of commercial flocks of U.S. chickens, usually with little mortality in the commercial flocks. These Low-Path H5N1 strains are very closely related in virus structure to their more lethal High-Path H5N1 relatives in Eurasia. A mutation from a Low-Path to a High-Path strain has so far not been observed but mutations of this type over time may be expected by one of skill in the art.

The tested vaccine was engineered to block both the entry site of H5N1 virus and the replication site of those H5N1 viruses that manage to enter into host cells. As such, the vaccine is called the TWO-PUNCH vaccine. As demonstrated below, evidence from the described test of the TWO-PUNCH vaccine in chickens suggests that both mechanisms for which the vaccine was designed were effective: (1) virus entry into inoculated chickens was diminished by immunity from the vaccine and (2) virus replication within infected cells was sufficiently limited by immunity from the vaccine to block excretion of the virus in feces of tested birds.

The TWO-PUNCH Replikins Vaccine is based on influenza Replikin peptides shared between influenza strains and conserved for decades within influenza strains. The vaccine was engineered as a mixture of twelve Replikin peptides identified as expressed from the genome of H5N1 virus. Six of the Replikin peptides are synthesized according to sequences isolated from the hemagglutinin protein of H5N1, which is involved in attachment and entry of influenza virus into a cell. Six of the Replikin peptides are synthesized according to sequences isolated from the pB1 gene area of H5N1, which has been identified as involved in replication of influenza virus in a host cell.

The following six Replikin sequences contained in the vaccine were isolated from the hemagglutinin protein:

| | |
|---|---|
| (1) HAQDILEKEHNGKILCSLKGVRPLILK; | (SEQ ID NO: 1) |
| (2) KEHNGKLCSLKGVRPLILK; | (SEQ ID NO: 2) |
| (3) KKNNAYPTIKRTYNNTNVEDLLIIWGIHH; | (SEQ ID NO: 3) |
| (4) HHSNEQGSGYAADKESTQKAIDGITNK; | (SEQ ID NO: 4) |
| (5) HDSNVKNLYDKVRLQLRDNAK; | (SEQ ID NO: 5) |
| and | |
| (6) KVRLQLRDNAKELGNGCFEFYH. | (SEQ ID NO: 6) |

The following six Replikin sequences contained in the vaccine were isolated from the pB1 gene area:

(SEQ ID NO: 7)

(1) KDVMESMDKEEMEITTH;

(SEQ ID NO: 8)

(2) HFQRKRRVRDNMTKK;

(SEQ ID NO: 9)

(3) KKWSHKRTIGKKKQRLNK;

(SEQ ID NO: 10)

(4) HKRTIGKKKQRLNK;

(SEQ ID NO: 14)

(5) HEGIQAGVDRFYRTCKLVGINMSKKK;
and

(SEQ ID NO: 12)

(6) HSWIPKRNRSILNTSQRGILEDEQMYQKCCNLFEK.

The vaccine comprises an approximate equal-parts-by-weight mixture of the twelve peptides. The following peptide amounts were combined to create an initial mixture of the vaccine:

| | | |
|---|---|---|
| HAQDILEKEHNGKLCSLKGVRPLILK | (SEQ ID NO: 1) | 239.6 mg |
| KEHNGKLCSLKGVRPLILK | (SEQ ID NO: 2) | 200.8 mg |
| KKNNAYPTIKRTYNNTNVEDLLIIWGIHH | (SEQ ID NO: 3) | 213.0 mg |
| HHSNEQGSGYAADKESTQKAIDGITNK | (SEQ ID NO: 4) | 135.6 mg |
| HDSNVKNLYDKVRLQLRDNAK | (SEQ ID NO: 5) | 170.8 mg |
| KVRLQLRDNAKELGNGCFEFYH | (SEQ ID NO: 6) | 188.3 mg |
| KDVMESMDKEEMEITTH | (SEQ ID NO: 7) | 161.9 mg |
| HFQRKRRVRDNMTKK | (SEQ ID NO: 8) | 138.3 mg |
| KKWSHKRTIGKKKQRLNK | (SEQ ID NO: 9) | 217.8 mg |
| HKRTIGKKKQRLNK | (SEQ ID NO: 10) | 178.0 mg |
| HEGIQAGVDRFYRTCKLVGINMSKKK | (SEQ ID NO: 11) | 159.2 mg |
| HSWIPKRNRSILNTSQRGILEDEQMYQKCCNLFEK | (SEQ ID NO: 12) | 233.8 mg |

The total amount of the mixture was 2237.1 mg.

The peptide mixture was then divided into three equal parts for administration of the vaccine on three different days (days 1, 7, and 28). After dissolution with water, the three equal parts were administered to individual birds in two groups of 20 birds each for a total administration at each day of 40 birds. The total amount of active peptide ingredient administered to each bird at the time of administration (either intranasally and intraocularly or via spray inhalation) was about 18.6 mg per bird per administration.

The vaccine solution was administered to chickens intranasally at a first administration on day 1 after hatch, intraocularly at a second administration on day 7 after hatch, and via fine spray inhalation at a third administration on day 14 after hatch.

Chickens on the first day of life were separated into four groups with twenty chickens per group. The first group was a control group not vaccinated and not challenged with Low-Path H5N1. The second group was vaccinated and not challenged with Low-Path H5N1. The third group was vaccinated and subsequently challenged with Low-Path H5N1. The fourth group was not vaccinated and was challenged with Low-Path H5N1.

For those chickens that were vaccinated, the synthetic H5N1 Replikins Vaccine was administered intranasally on day 1 after hatch, administered intraocularly on day 7 after hatch, and administered by fine spray inhalation on day 14 after hatch. The groups of challenged chickens were than challenged with Low-Path H5N1 virus on day 28 of the life of the chicken. Serum from selected chickens was analyzed in all groups for antibodies against the H5N1 virus on days 7, 14, and 21 following challenge. PCR for virus fecal excretion was also analyzed for all groups.

Unvaccinated control chickens demonstrated both an expected high virus entry (as indicated by a high titer of antibodies against H5N1) and an expected high virus replication (as indicated by high fecal and salival excretion of the virus detected by PCR). In contrast, the vaccinated chickens demonstrated lower virus entry (as indicated by a low titer of antibodies against H5N1 or by the observation of no antibodies against H5N1 in serum) and an absence of fecal or saliva excretion of virus indicating low or no virus replication in the vaccinated chickens. The data suggest, therefore, that the virus was partially blocked on entry by the chickens' immune response to the vaccine and the limited amount of virus that did enter the chicken's system was blocked from sufficient replication in the chickens' host cells to excrete virus in the feces or saliva.

The data in Table 14 below provide the numbers of chickens tested in each of the four groups (Negative Control, Vaccinated, Vaccinated and Challenged with Low-Path H5N1, and Challenged with Low-Path H5N1 (not vaccinated)) on a particular test day and the numbers of chickens in which production of antibodies to H5N1 was detected with a serum titer.

TABLE 14
Serum Antibody Test of Low-Path H5N1
Challenge of Vaccinated Chickens

| | Day 7 | Day 14 | Day 21 |
|---|---|---|---|
| | (Chickens | (Chickens | (Chickens |
| | Producing | Producing | Producing |
| | Antibody | Antibody | Antibody |
| GROUP | to H5N1) | to H5N1) | to H5N1) |
| Negative Control | 0 of 7 | 0 of 7 | 0 of 7 |
| Vaccinated | 0 of 7 | 6 of 6 | 0 of 5 |
| Vaccinated and | 1 of 7 | 3 of 6 | 2 of 7 |
| Challenged with Low- | | | |
| Path H5N1 | | | |
| Challenged with Low- | 4 of 7 | 7 of 9 | 3 of 9 |
| Path H5N1 | | | |

The data in Table 15 below provide the number of chickens tested for H5N1 virus in their saliva and feces in each of the four groups (Negative Control, Vaccinated, Vaccinated and Challenged with Low-Path H5N1, and Challenged with Low-Path H5N1 (not vaccinated)) on a particular test day and the numbers of chickens in which H5N1 was detected in their feces and saliva based on PCR analysis.

TABLE 15
PCR Test for Excreted H5N1 Virus from Low-Path
H5N1 Challenge of Chickens

| | Day 7 | Day 14 | Day 21 |
|---|---|---|---|
| | (Chickens | (Chickens | (Chickens |
| | Producing | Producing | Producing |
| | Antibody | Antibody | Antibody |
| GROUP | to H5N1) | to H5N1) | to H5N1) |
| Negative Control | 0 of 10 | 0 of 7 | 0 of 7 |
| Vaccinated | 0 of 10 | 0 of 7 | 0 of 7 |
| Vaccinated and | 0 of 7 | 0 of 7 | 0 of 7 |
| Challenged with Low- | | | |
| Path H5N1 | | | |
| Challenged with Low- | 3 of 7 | 2 of 9 | 1 of 7 |
| Path H5N1 | | | |

The data in Tables 14 and 15, demonstrate the effectiveness of the double-protective mechanism of the TWO-PUNCH vaccine. First, while several non-vaccinated chickens challenged with H5N1 excreted virus in their feces and saliva, no vaccinated chickens challenged with H5N1 excreted virus in their feces or saliva. See Table 15. These data demonstrate that the vaccine provided a protective effect against replication of the virus. Second, while four of seven unvaccinated chickens challenged with H5N1 were producing serum antibodies against H5N1 on day 7, seven of nine unvaccinated chickens challenged with H5N1 were producing serum antibodies against H5N1 on day 14, and three of nine unvaccinated chickens challenged with H5N1 were producing serum antibodies against H5N1 on day 28, only one of seven vaccinated and challenged chickens was producing serum antibodies against H5N1 on day 7, only three of six vaccinated and challenged chickens were producing serum antibodies against H5N1 on day 14, and only two of seven vaccinated and challenged chickens were producing serum antibodies against H5N1 on day 21. See Table 14. These data demonstrate that for some of the vaccinated chickens, the H5N1 virus challenge was stopped prior to entry into the chicken's system (likely by antibodies produced at the mucus membranes). These data further demonstrate that for those vaccinated and challenged chickens in which the virus entered the system (resulting in production of serum antibodies), the virus was nonetheless not excreted in feces or saliva.

As may be seen from the data in Table 14, almost all of the non-vaccinated challenged birds seroconverted (producing detectable antibody). This demonstrates infection of the non-vaccinated birds. On the other hand, only some of the vaccinated challenged birds seroconverted. Further, for those vaccinated birds that did seroconvert, the antibody titers were low. Additionally, the negative control group had no seroconversion. These data demonstrate a protective effect of the vaccine on the birds.

Additionally, Table 15 demonstrates the absence of detectable influenza in the feces and saliva of vaccinated birds. That viral excretion was blocked by this influenza Replikins vaccine is particularly significant because it is generally acknowledged that the maintenance of reservoirs of H5N1 virus in flocks of migratory birds and domestic chickens in both Asia and the U.S. (and the regional spread of H5N1 virus from these reservoirs) is dependent on viral excretions picked up by neighboring chickens and birds. Regardless of the level of lethality of a strain of H5N1 virus, absent excretion of virus, there is expected to be no spread of the virus.

As such, data observed from administration of the TWO-PUNCH Replikin peptide vaccine in chickens demonstrates the efficacy of the vaccine as (1) a barrier to entry of the virus, (2) a block of replication of the virus, and (3) a block of fecal spread of the virus.

What is claimed is:

1. A method of predicting an expansion of a population of a first pathogen comprising:
identifying at least one cycle of Replikin concentration in isolates of the first pathogen; and
predicting that an expansion of the population of the first pathogen will take place after the occurrence of at least one rising portion of the at least one cycle of Replikin concentration, wherein the at least one cycle is cycle A.

2. The method of claim 1, wherein the at least one rising portion comprises a peak and wherein said expansion of the population of the first pathogen is predicted after the occurrence of the peak.

3. The method of claim 1, wherein said at least one rising portion comprises at least a first rising portion and a second rising portion, wherein said first rising portion occurs prior in time to said second rising portion.

4. The method of claim 1, wherein said at least one rising portion comprises at least rising portion A′, rising portion B′ and rising portion C′.

5. The method of claim 4, wherein said rising portion B′ comprises a peak B and said rising portion A′ comprises a peak A, and wherein the peak B of rising portion B′ has a greater Replikin concentration than the peak A of rising portion A′.

6. A method of predicting an expansion of a population of a pathogen comprising:
identifying at least one cycle of the Replikin concentration of a plurality of isolates of the pathogen or of a related pathogen,
identifying a first peak in the Replikin concentration of a plurality of isolates of said pathogen within the at least one identified cycle at a first time point or time period, and
predicting that an expansion of the population of a pathogen of the same species that is isolated at a second time point or time period will occur subsequent to the first time point or time period.

7. The method of claim 1, wherein said first pathogen is a malarial trypanosome, a West Nile virus, a foot and mouth disease virus, or an influenza virus.

8. The method of claim 1, wherein said first pathogen is an H5N1 strain of influenza virus.

9. The method of claim 1, further comprising:
identifying at least one other cycle of Replikin concentration in isolates of at least one other strain of pathogen, wherein the at least one other cycle is cycle B, and wherein cycle B shares synchrony with cycle A; and
predicting that an expansion of the population of the first pathogen will occur after the occurrence of the at least one rising portion in cycle A, wherein the at least one rising portion in cycle A corresponds to a rising portion in cycle B.

10. The method of claim 9, wherein said first pathogen is a strain of influenza virus and wherein said at least one other strain of pathogen is a different strain of influenza virus.

11. The method of claim 9, wherein said first pathogen is an H5N1 strain of influenza virus and said at least one other strain of pathogen is an H9N2 strain of influenza virus.

12. The method of claim 2, wherein said expansion of the population of the first pathogen is predicted within three years after said peak.

13. The method of claim 2, wherein said expansion of the population of the first pathogen is predicted within one year after said peak.

14. The method of claim 2, wherein said expansion of the population of the first pathogen is predicted after a next virulence season of the pathogen.

15. An isolated or synthesized protein, protein fragment, or peptide comprising a Replikin peptide or a Replikin Peak Gene of a pathogen, wherein said pathogen is predicted to have an expansion of the population of the pathogen in accordance with the method of claim 1.

16. The isolated or synthesized protein, protein fragment, or peptide of claim 15 consisting of one or more Replikin peptides and/or one or more Replikin Peak Genes.

17. The isolated or synthesized protein, protein fragment, or peptide of claim 16, wherein said one or more Replikin peptides are conserved during the at least one cycle in Replikin concentration at least two successive time points or time periods in the at least one cycle.

18. The isolated or synthesized protein, protein fragment, or peptide of claim 17 comprising at least one of peptide of HAQDILEKEHNGKLCSLKGVRPLILK (SEQ ID NO: 1), KEHNGKLCSLKGVRPLILK (SEQ ID NO: 2), KKNNAYPTIKRTYNNTNVEDLLIIWGIHH (SEQ ID NO: 3), HHSNEQGSGYAADKESTQKAIDGITNK (SEQ ID NO: 4), HDSNVKNLYDKVRLQLRDNAK (SEQ ID NO: 5), KVRLQLRDNAKELGNGCFEFYH (SEQ ID NO: 6), KDVMESMDKEEMEITTH (SEQ ID NO: 7), HFQRKRRVRDNMTKK (SEQ ID NO: 8), KKWSHKRTIGKKKQRLNK (SEQ ID NO: 9), HKRTIGKKKQRLNK (SEQ ID NO: 10), HEGIQAGVDRFYRTCKLVGINMSKKK (SEQ ID NO: 11); or HSWIPKRNRSILNTSQRGILEDEQMYQKCCNLFEK (SEQ ID NO: 12).

19. An immunogenic composition comprising the isolated or synthesized protein, protein fragment, or peptide of claim 15.

20. The immunogenic composition of claim 19, wherein said isolated or synthesized protein, protein fragment, or peptide consists of a Replikin peptide or a Replikin Peak Gene.

21. The immunogenic composition of claim 20, wherein said Replikin peptide or said Replikin Peak Gene are conserved during at least one of the at least one cycle in Replikin concentration at least two successive time points or time periods in the at least one of the at least one cycle.

22. A method of preventing, mitigating, or treating an outbreak of a first pathogen predicted to have an expansion of population comprising:
predicting the expansion of the population of the first pathogen in accordance with the method of claim 1; and
administering to an animal or patient a compound comprising an isolated or synthesized portion of the structure or genome of the first pathogen to mitigate, prevent, or treat the predicted outbreak of the first pathogen.

23. A method of making a vaccine comprising:
predicting the expansion of the population of the first pathogen in accordance with claim 1; and
identifying a portion of the structure or genome of said first pathogen to be comprised in a vaccine.

24. A computer readable medium having stored thereon instructions which, when executed, cause a processor to perform the method of predicting the expansion of the population of the first pathogen of claim 1.

25. The computer readable medium of claim 24 wherein said method of predicting an expansion of the population of the first pathogen of claim 1 further comprises outputting the prediction to a display, user, researcher, or other machine or person.

26. The computer readable medium of claim 24 further comprising instructions stored thereon which, when executed, cause a processor to identify to a display, user, researcher, or other machine or person, at least one Replikin peptide of said first pathogen that is conserved in said first pathogen.

27. A method of predicting an expansion of a strain of pathogen comprising:
determining a mean Replikin Count and a standard deviation of said mean Replikin Count for a plurality of isolates of a strain of pathogen for a first time period in a first geographic region;
determining a Replikin Count of at least one isolate of the same or a related strain of pathogen from a second time period and/or second geographic region, wherein said second time period is different from said first time period and/or said second geographic region is different from said first geographic region, and wherein the second time period is not necessarily after the first time period; and
predicting an expansion of said strain of pathogen isolated in said second time period and/or said second geographic region if the Replikin Count of said at least one isolate is greater than one standard deviation of the mean of the Replikin Count of the plurality of isolates isolated in said first time period and in said first geographic region.

28. The method of claim 27, wherein the at least one isolate of the same strain of pathogen from a second time period and/or second geographic region is a plurality of isolates from said second time period and/or said second geographic region, and the Replikin Count of each isolate of the plurality of isolates from said second time period and/or second geographic region is compared separately to said one standard deviation of said mean Replikin Count.

29. The method of claim 28, wherein the expansion of said strain of pathogen isolated in said second time period and/or said second geographic region is predicted if the number of Replikin Counts of said plurality of isolates from said second period and/or said second geographic region that is greater than one standard deviation of the mean of the Replikin Count of the plurality of isolates isolated in said first time period in said first geographic region, is greater than the number of Replikin Counts of said plurality of isolates from said second time period and/or said second geographic region that is less than said one standard deviation of the mean.

30. The method of claim 27, wherein said pathogen is an influenza virus, a malarial trypanosome, a West Nile virus, or a foot and mouth disease virus.

31. The method of claim 27, wherein said first time period is one year and said first geographic region is a country.

32. The method of claim 31, wherein said second time period is one year and said second geographic region is a country.

33. A method of preventing, mitigating, or treating an outbreak of a pathogen comprising predicting an expansion of a strain of pathogen in accordance with claim 27 and administering to an animal or a patient a compound comprising an isolated or synthesized portion of the structure or genome of the at least one isolate of pathogen to prevent or treat the outbreak of the pathogen.

34. The method of claim 1, wherein said method is performed by a computer.

35. The method of claim 27, wherein said method is performed by a computer.

36. The method of claim 6, wherein said method is performed by a computer.

37. The method of claim 28, wherein said method is performed by a computer.

38. The method of claim 6, wherein said pathogen is a malarial trypanosome, a West Nile virus, a foot and mouth disease virus, or an influenza virus.

39. The method of claim 6, wherein said pathogen is an H5N1 strain of influenza virus.

40. A quantitative cyclic structure comprising Replikin peptide concentrations identified in a strain of microorganism through time, wherein said cyclic structure correlates in time with the expansion and/or contraction of a population of said strain of microorganism, the infectivity of said strain of microorganism, and/or the lethality of said strain of microorganism in its host.
